(19) United States
(12) Patent Application Publication
(10) Pub. No.: US 2026/0009071 A1
(43) Pub. Date: Jan. 8, 2026

(54) METHODS OF IN-SOLUTION POSITIONAL CO-BARCODING FOR SEQUENCING LONG DNA MOLECULES

(71) Applicant: MGI Tech Co., Ltd., Shenzhen (CN)

(72) Inventors: Andrei Alexeev, Woodland, CA (US); Brock A. Peters, San Jose, CA (US); Radoje T. Drmanac, Los Altos Hills, CA (US)

(21) Appl. No.: 18/998,483

(22) PCT Filed: Jul. 20, 2023
(86) PCT No.: PCT/CN2023/108314

§ 371 Date: Jan. 25, 2025

Related U.S. Application Data
(60) Provisional application No. 63/369,346, filed on Jul. 25, 2022.

(51) Int. Cl. C12Q 1/6874

(52) U.S. Cl. CPC C12Q 1/6874

(57) ABSTRACT

The methods and compositions disclosed herein relate to preparing libraries to sequence long molecules in their entirety using massively parallel short read sequencing. The methods disclosed herein generate a nested set of nucleic acid constructs for each genomic fragment and generate a plurality of nested sets for a plurality of genomic fragments. The nucleic acid constructs may be single-stranded or double-stranded. Each nucleic acid construct in each nested set comprises a barcode and target sequence portion, and nucleic acid constructs within each nested set have different lengths. The nucleic acid constructs in each nested set share a unique barcode sequence.

### RELATED APPLICATIONS

This application claims priority to U.S. Provisional Application No. 63/369,346, filed on Jul. 25, 2022. The entire content of the provisional application is herein incorporated by reference for all purposes.

### BACKGROUND

Sequencing long genomic DNA can be challenging. Many sequencing platforms, such as DNBseq sequencers and Illumina sequencers, are not designed to sequence long DNA molecules. For example, it may be difficult to produce DNBs from long DNA molecules with enough copies of the templates for high-quality sequencing in DNBseq sequencers. Illumina sequencers typically require bridge amplification, and bridge amplification of long DNA molecules tends to be inefficient. In addition, the length of reads possible using these systems are typically less than 500 bases and so the middle of these molecules cannot be sequenced. Thus, although these MPS sequencing platforms can be cost-effective and efficient, the sequence reads obtained from these platforms are limited in length. Additional challenges exist for sequencing DNA molecules with highly repetitive nucleotide sequences; for example, it is often difficult to decipher whether two identical sequence reads are associated with different positions in the genome, or whether they are simply duplicate sequence reads of the same position in the genome. Thus, there remains a need to prepare sequencing libraries such that the sequence information of long DNA molecules can be gleaned from short sequence reads in an accurate and efficient manner.

### SUMMARY OF INVENTION

In one aspect, disclosed herein are methods of producing single-stranded adaptered constructs for sequencing, optionally without the use of nanodrops, comprising: preparing a plurality of nested sets of single-stranded nucleic acid constructs, optionally in a single mixture, wherein each single-stranded nucleic acid construct in each nested set comprises a target sequence portion flanked by a first adapter sequence at the 5′ end and a second adapter sequence at the 3′ end, wherein the first adapter sequence comprises, from 5′ to 3′, a primer-binding sequence, a barcode sequence and a first hybridization sequence and the second adapter sequence comprises a second hybridization sequence, wherein the first and the second hybridization sequences are complementary to each other, wherein each target sequence portion has a first end and a second end, wherein the distance between the first end and the barcode sequence is shorter than the distance between the second end and the barcode sequence, wherein the single-stranded nucleic acid constructs in each nested set share the same barcode sequence and the single-stranded nucleic acid constructs in different nested sets have different barcode sequences, and wherein for each nested set of single-stranded nucleic acid constructs, the target sequence portions in that nested set have identical nucleotide sequences near the first ends and differ from each other by truncations near the second ends, such that each nested set of single-stranded nucleic acid constructs comprises a plurality of target sequence portions having different lengths.

In another aspect, disclosed herein is a method of producing single-stranded DNA circles comprising single-stranded adaptered constructs for sequencing, optionally without the use of nanodrops, comprising: preparing a plurality of nested sets of single-stranded nucleic acid constructs in a single mixture, wherein each single-stranded nucleic acid construct in each nested set comprises a target sequence portion flanked by a first adapter sequence and a second adapter sequence, wherein the first adapter sequence comprises a barcode sequence and a primer-binding sequence, wherein each target sequence portion has a first end and a second end, wherein the distance between the first end and the barcode sequence is shorter than the distance between the second end and the barcode sequence, wherein the single-stranded nucleic acid constructs in each nested set share the same barcode sequence and the single-stranded nucleic acid constructs in different nested sets have different barcode sequences, wherein for each nested set of single-stranded nucleic acid constructs, (a) the target sequence portions in that nested set have identical nucleotide sequences near the first ends and differ from each other by truncations near the second ends, such that each nested set of single-stranded nucleic acid constructs comprises a plurality of target sequence portions having different lengths, and (b) circularizing the single-stranded nucleic acid constructs in each nested set to produce the single-stranded DNA circles, in which the first adapter sequence and the second adapter sequence are joined.

In another aspect, disclosed herein is a method of producing single-stranded DNA circles comprising single-stranded adaptered constructs for sequencing, optionally without the use of nanodrops, comprising: in a single mixture, preparing a plurality of nested sets of single-stranded nucleic acid constructs, wherein each single-stranded nucleic acid construct in each nested set comprises a target sequence portion flanked by a first adapter sequence and a second adapter sequence, wherein the first adapter sequence comprises a barcode sequence and a primer-binding sequence, wherein each target sequence portion has a first end and a second end, wherein the distance between the first end and the barcode sequence is shorter than the distance between the second end and the barcode sequence, wherein the single-stranded nucleic acid constructs in each nested set share the same barcode sequence and the single-stranded nucleic acid constructs in different nested sets have different barcode sequences, wherein for each nested set of single-stranded nucleic acid constructs, (a) the target sequence portions in that nested set have identical nucleotide sequences near the first ends and differ from each other by truncations near the second ends, such that each nested set of single-stranded nucleic acid constructs comprises a plurality of target sequence portions having different lengths, and (b) circularizing the single-stranded nucleic acid constructs in each nested set to produce the single-stranded DNA circles, in which the first adapter sequence and the second adapter sequence are joined.

In another aspect, disclosed herein is a method of producing double-stranded adaptered constructs for sequencing, optionally without the use of nanodrops, wherein the method comprises: (i) amplifying a plurality of genomic fragments, each genomic fragment comprising a target sequence, to produce a plurality sets of amplified nucleic acid fragments in a mixture, wherein the amplified nucleic acid fragments in each set share the same target sequence, optionally the amplification is performed using target-specific primers, for each set, the method further comprises (ii) contacting the amplified nucleic acid fragments with an enzyme, wherein the enzyme introduces breaks in the amplified nucleic acid fragments, (iii) distributing the mixture of fragments into a plurality of aliquots, (iv) performing nick translation on the aliquots of fragments to synthesize DNA strands under conditions such that the DNA strands synthesized in different aliquots have different lengths, wherein each of the DNA strands comprises a target sequence portion with a first end and a second end, and wherein the DNA strands in different aliquots share the same sequence near the first ends and have different sequence near the second ends, (v) for each aliquot, ligating second adapters to the second ends of the DNA strands synthesized in (iv) via branch ligation, wherein each second adapter is a partially double stranded adapter comprising a first adapter oligonucleotide and a second adapter oligonucleotide, wherein both the first adapter oligonucleotide and a second adapter oligonucleotide are complementary and hybridized to each other, wherein each of the second adapters comprises a positional barcode sequence, wherein each ligation comprises joining a 5-prime end of the first adapter oligonucleotide of the second adapter to a second end of the synthesized DNA strand, wherein the first adapter oligonucleotides ligated to the second ends of the synthesized DNA strands in different aliquots comprise different positional barcode sequence, and the first adapter oligonucleotides ligated to the second ends of the synthesized DNA strands in the same aliquot share the same positional barcode sequence, (vi) combining the synthesized DNA strands ligated with the second adapters from different aliquots from (v) in a single mixture, (vii) extending a primer hybridized to the first adapter oligonucleotides that have been ligated to the synthesized DNA strands to produce double-stranded fragments having blunt ends, and (viii) optionally selecting the double-stranded fragments of (vii) with a size within a range from 200 bp-1.5 kb, for example, 300 bp-1.2 kb, 300 bp-1 kb, or 500-1000 bp, from the single mixture, and (ix) ligating a third adapter to the blunt ends of the double-stranded fragments, thereby producing double-stranded adaptered constructs.

In another aspect, disclosed herein is a method for preparing a plurality of nested sets of adaptered fragments, optionally without the use of nanodrops, wherein each adaptered fragment is a single-stranded nucleic acid comprising a target sequence fragment having a first end and a second end, a 5-prime adapter sequence, a 3-prime adapter sequence, a primer sequence, and a barcode sequence, wherein in each nested set of adaptered fragments, the target sequence fragments have identical nucleotide sequences at the first end and differ from each other by truncations at the second end, such that each nested set of adaptered fragments comprises a plurality of target sequence fragments having different length, wherein the first end is closer to the barcode sequence than the second end, wherein the method comprises: (a) providing, in a reaction of a single mixture, a population of single-stranded DNA concatemers, wherein each concatemer comprises a plurality of identical monomers, and each monomer comprises a complement of a target sequence, a complement of the barcode sequence that identifies the concatemer, and a primer-binding sequence shared by the population of single-stranded concatemers, wherein the primer-binding sequence comprises a sequence that is complementary to the primer sequence, wherein both the primer-binding sequence and complement of the barcode sequence are 3-prime to the complement of the target sequence; (b) annealing primers comprising the primer sequence to primer-binding sequences of multiple monomers of each of plurality of the concatemers; (c) extending at least some of the primers hybridized to the primer-binding sequences with a DNA polymerase that has 5′-->3′ exonuclease activity and does not have strand displacement activity, wherein the extending produces a plurality of extended primers, each said extended primer comprising a target sequence fragment with barcode sequences and primer sequences, wherein the extended primers are hybridized to the concatemer; wherein the extended primers are separated by intervals, and (d) contacting the plurality of the extended primers with a 5-prime adapter comprising the 5-prime adapter sequence, a 3-prime adapter comprising the 3-prime adapter sequence, a DNA ligase, and an exonuclease having single-strand DNA exonuclease activity under conditions in which the exonuclease degrades a portion of the target sequence fragments in the extended primers, to produce shortened extended primers, the 5-prime adapters are ligated to the 5′ end of the shortened extended primers, and the 3-prime adapters are ligated to the 3′ end of the shortened extended primers, thereby producing a group of plurality of nested sets of adaptered fragments.

In another aspect, disclosed herein is a method for preparing a plurality of nested sets of adaptered fragments, optionally without the use of nanodrops, wherein each adaptered fragment is a single-stranded nucleic acid comprising a target sequence fragment having a first end and a second end, a 5-prime adapter sequence, a 3-prime adapter sequence, a primer-binding sequence, and a complement of a barcode sequence, wherein in each nested set of adaptered fragments, the target sequence fragments have identical nucleotide sequences at a first end and differ from each other at a second end, such that each nested set of adaptered fragments comprises a plurality of target sequence fragments having different length, wherein the first end is closer to the barcode sequence than the second end, wherein the method comprises (a) providing a barcoded fragment comprising a barcode sequence, a target sequence, and a primer binding sequence, wherein the barcoded fragment is immobilized on a bead at one terminus, (b) annealing a primer comprising the 5-prime adapter sequence to the primer-binding sequence in the barcoded fragment, wherein the 5-prime adapter sequence comprises i) a complement of the barcode sequence, and ii) a primer sequence complementary to the primer binding sequence in the barcoded fragment, (c) extending the primer to produce an extended primer comprising a target sequence fragment and a complement of the barcode sequence, (d) contacting the extended primer with a branch adapter comprising the 3-prime adapter sequence to produce an adaptered fragment, (e) separating the adaptered fragment from the barcoded fragment that remains immobilized on the bead, and (f) repeating steps (b)-(e) for one or more cycles under extension-controlling conditions to produce one or more adaptered fragments, wherein the adaptered fragment generated from step (e) and the adaptered fragments generated from step (f) and constitute the nested set of adaptered fragments, and wherein the adaptered fragments in each nested set comprise target sequence fragments having different length.

In another aspect, disclosed herein is a method for preparing a plurality of sets of adaptered fragments, optionally without the use of nanodrops, wherein each adaptered fragment is a single-stranded nucleic acid comprising a target sequence fragment having a first end and a second end, a 5-prime adapter sequence, a 3-prime adapter sequence, a primer-binding sequence, and a complement of a barcode sequence, wherein the method comprises, (a) providing a barcoded fragment comprising a barcode sequence, a target sequence, and a primer binding sequence, wherein the barcoded fragment is immobilized on a bead at one terminus, (b) annealing a primer comprising the 5-prime adapter sequence to the primer-binding sequence in the barcoded fragment, wherein the 5-prime adapter sequence comprises i) a complement of the barcode sequence, and ii) a primer sequence complementary to the primer binding sequence in the barcoded fragment, (c) extending the primer to produce an extended primer comprising a target sequence fragment and the complement of the barcode sequence, (d) contacting the extended primer with a first branch adapter comprising a 3-prime portion comprising a degenerate sequence region, thereby forming a first extension product comprising the degenerate sequence region at the 3-prime portion, wherein the 3-prime portion is hybridized to the barcoded fragment through the degenerate sequence region, (e) extending the 3-prime portion of the first extension product to generate a second extension product, and (f) contacting the second extension product with a second branch adapter to produce the adaptered fragment.

In another aspect, disclosed herein is a DNA complex comprising (a) a barcoded fragment immobilized on a solid support, wherein the barcoded fragment comprises a barcode sequence and a target sequence, and (b) a polynucleotide hybridized to the barcoded fragment, wherein the polynucleotide comprises a 5-prime portion comprising a complement of the barcode sequence, a 3-prime portion comprising a target sequence fragment, wherein the 5-prime portion and the 3-prime portion are annealed to the barcoded fragment, leaving a middle portion not annealed to the barcoded fragment, thereby forming a bubble.

In another aspect, disclosed herein is a composition comprising a nested set of adaptered fragments each comprising a barcode sequence and a target sequence fragment having a first end and a second end, a 5-prime adapter sequence, and a 3-prime adapter sequence, wherein the target sequence fragments have identical nucleotide sequences at the first end and differ from each other by truncations at the second end, such that the nested set of adaptered fragments comprises a plurality of target sequence fragments having different length, and wherein the nested set of adaptered fragments share same barcode sequence.

### BRIEF DESCRIPTION OF THE DRAWINGS

The drawings and descriptions thereof illustrate exemplary embodiments of the disclosure. The methods and compositions provided in this disclosure are not limited to the embodiments shown in these drawings.

FIG. 1 shows an embodiment of a method in this disclosure. The top panel represents an adaptered double-stranded genomic fragment, which comprises a target sequence with a first end and a second end. The target sequence is flanked by adapter 1 at the 3-prime adapter 3 at the 5-prime. The first adapter comprises a primer binding site and a barcode sequence, the primer binding site located 3-prime relative to the barcode sequence (not shown).

FIG. 2 shows one exemplary embodiment of a method in this disclosure. Various methods steps are shown including adding barcodes to genomic fragments and amplifying the barcoded genomic fragments.

FIG. 3A shows one embodiment of the DNA circle-based scheme to produce single-stranded DNA circles from the amplified genomic fragments in FIG. 2.

FIG. 3B shows another embodiment of the DNA circle-based scheme to produce single-stranded DNA circles from the amplified genomic fragments in FIG. 2.

FIG. 3C shows another embodiment of the DNA circle-based scheme to produce single-stranded DNA circles from the amplified genomic fragments in FIG. 2.

FIG. 4A shows one embodiment of the DNA circle-based scheme to produce double-stranded adaptered constructs from the single-stranded DNA circles formed as shown in FIG. 3A or FIG. 3B.

FIG. 4B shows another embodiment of the DNA circle-based scheme to produce double-stranded adaptered constructs from the single-stranded DNA circles formed as shown in FIG. 3A or FIG. 3B.

FIG. 5 shows one embodiment of the linear DNA-based scheme to produce double-stranded adaptered constructs for sequencing.

FIGS. 6A and 6B show a concatemer-based method of the invention. FIG. 1A shows that a double-stranded DNA molecule comprising a barcode 110 and a target sequence 120 is denatured to single-stranded nucleic acid. The single-stranded nucleic acid is circularized and amplified by rolling circle replication forming a concatemer comprising multiple monomers, each comprising a complement of a target nucleic acid sequence, a complement of the barcode sequence 121, and a primer-binding sequence 131. In each monomer, a primer 130 is annealed to a primer-binding sequence 131 that is 3-prime relative to the complement of the barcode sequence 111 and extended using a polymerase having no strand-displacement activity but having 5-prime to 3-prime exonuclease activity. The extended primer 150 are separated by intervals 160. Optionally, the intervals 160 are widened by a gapping enzyme, which results in gaps 170. If the primer 130 is an RNA primer, then the gapping enzyme can be RNase H. An L-adapter is then ligated to the 5-prime of the extended primer, and a branch-adapter is then ligated to the 3-prime of the extended primer in the presence of a 3-prime to 5-prime exonuclease. Note that although extension, gapping, and ligations are shown in separate steps, in some embodiments, reagents that are used for one or more of these reactions can be added simultaneously into a single reaction mixture. After denaturing, a nested set of single-stranded, adaptered fragments 191 having different lengths of target sequence fragments (122-125) are produced, each having an L-adapter sequence at the 5-prime and a branch adapter sequence at the 3-prime. The barcodes are located at the 5-prime portion of the adaptered fragments.

FIG. 7A-7B illustrate another concatemer-based method of the invention. Similar to FIG. 6, a polymerase is used to extend a primer 230 annealed to the primer binding sequence 231. The DNA polymerase has no strand-displacement activity but having 5-prime to 3-prime exonuclease activity. 210 is the barcode and 220 is the target sequence. Unlike FIG. 6, the primer binding sequence 231 is 5-prime relative to the complement of the barcode sequence 211 (the primer binding sequence 131 is 3-prime relative to the complement of the barcode sequence 111 in FIG. 6). Also unlike FIG. 6, the ligations of the L-adapter 280 and the branch adapter 290 are performed in the presence of a 5-prime to 3-prime exonuclease (instead of a 3-prime to 5-prime exonuclease as in FIG. 6). After denaturing, a nested set of adaptered fragments 291 are formed each having an L-adapter sequence at the 5-prime and a branch adapter sequence at the 3-prime. The adaptered fragments comprise target sequence fragments 222, 223, 224, and 225. These target sequence fragments are produced with different lengths. The barcodes 210 are located at the 3-prime portion of the adaptered fragments.

FIG. 8 shows an embodiment of the combinational scheme-based method of the invention. Genomic DNA are first fragmented to generate staggered fragments having single-stranded breaks as disclosed FIG. 2 in U.S. Provisional Application No. 63/224,731. StLFR is performed to produce co-barcoded fragments comprising a branch adapter sequence at one terminus and an L-adapter sequence at the other terminus. These co-barcoded fragments are then released from the beads and are then circularized and processed according to procedures described in FIG. 6A-6B or FIG. 7A-7B.

FIG. 9 shows another exemplary embodiment of the combinational scheme-based method of the invention. A barcoded fragment comprising a barcode sequence 410, a primer binding sequence 433, and a target sequence 420, is immobilized on a bead, and the 3-prime terminus of the barcoded fragment is also immobilized on a bead. The tailed primer comprises a tail 431 (which is optional), which is not hybridized to the barcode fragment. The tailed primer comprises a primer sequence 432, which hybridizes to the primer binding sequence 433, and a complement of the barcode sequence 411, which hybridizes to the barcode sequence in the barcoded fragment. The tailed primer is extended to produce an extended tailed primer 435, which comprises a target sequence fragment 436 and a complement of the barcode sequence 411. A branch adapter 440 is ligated to the 3-prime terminus of the target sequence fragment 436 to produce an adaptered fragment 450. The adaptered fragment 450 is then separated from the barcoded fragment 460, which remains immobilized on the bead. The barcoded fragment is then used as template for subsequent cycle of extension of tailed primer 430 to produce a plurality of extended tailed primers comprising target sequence fragments 451-453. The extensions were controlled such that the target sequence fragments 451-453 have different lengths.

FIG. 10A-10C show another exemplary embodiment of the combinational scheme-based method. The barcoded fragment and the tailed primer are provided as described in FIG. 9. FIG. 10A shows that after an initial period of extension with normal deoxynucleotides, uracils are added to the reaction mixture to produce an extended tailed primer 550 comprising uracils and followed by adding normal deoxynucleotides (e.g., uracil-free deoxynucleotides) and reversible terminators. The terminators can be added at different concentrations at different cycles in order to produce extended tailed primers comprising target sequence fragments having different lengths. FIG. 10B shows that the extended tailed primer 550 is then ligated to a branch adapter 540 to produce the adaptered fragment 551. The reversible terminators, if used, must be reversed before the ligation. The adaptered fragment 551 is then digested by USER to remove uracil, which leaves an interval 560 flanked by an exposed 3-prime terminus 570 and an exposed 5-primer terminus 580. An internal branch adapter 551 is then ligated to the exposed 3-prime terminus 570, and an L-adapter 552 is then ligated to the exposed 5-prime terminus 580 in the gap. A splint oligo 590 is then hybridized to 3-prime portion of the internal branch adapter 551 and the 5-prime portion of the L adapter 552 to allow the ligation between the two. The ligation which results in a shortened adaptered fragment 600 and a loop 591 in the barcoded fragment (still immobilized). The shortened adaptered fragment 600 can be separated from the barcoded fragment upon denaturation.

FIG. 10C shows fragments produced from repeating the process depicted in FIG. 10B for multiple cycles. Each shortened adaptered fragment comprises a shortened target sequence fragment (e.g., 610 or 620, or 630) produced from different cycles.

FIG. 11 shows the shortened target sequence fragments, e.g., 610, 620, and 630, have sequences that correspond to different regions of the target sequence produced from the process depicted in FIG. 10A-10C. Since all shortened adaptered fragments comprise the same complement of barcode sequence 511, sequencing reads from these adaptered fragments can be assembled based on the shared barcode sequence to achieve complete coverage across along target sequence.

FIG. 12 show another exemplary embodiment of the combinational scheme-based method. A primer annealed to the primer-binding sequence in the barcoded fragment is extended in a first extension under the extension-controlling conditions. The extended primer is ligated to a branch adapter 720 having a degenerate sequence region 730 at the 3-prime portion. The first branch adapter can hybridize to random locations in the barcoded fragment through the degenerate sequence region, which forms a loop 740 and result in skipping of replication of some random portion of the barcoded fragment. A second extension is then performed by extending the 3-prime terminus of the first branch adapter 750 to form a second extension product under extension-controlling conditions. A second branch adapter is ligated to the 3-prime terminus of the second extension product to produce an adaptered fragment. 710 is the barcode. 711 is the complement of the barcode.

FIGS. 13A and 13B show another embodiment of the invention of preparing a nested set of target sequence fragments for the loop-mediated complete stLFR.

FIG. 14 shows an embodiment of the loop-mediated complete stLFR.

FIG. 15 shows an embodiment of preparing the molecules produced from the loop-mediated complete stLFR shown in FIG. 14 for sequencing.

### DETAILED DESCRIPTION

### 1. Overview

The methods disclosed herein relate to preparing libraries to sequence long molecules in their entirety using massively parallel short-read sequencing. These long DNA molecules typically have a length in the range of 1-20 kb, for example, over 1000 bp, or over 1500 bp or over 2000 bp, or over 3,000 bp). These strategies disclosed herein do not require clonally barcoded beads and can be performed completely in solution, i.e., the genomic fragments and the adapters are all in solution during the entire library preparation. Thus, they can be conveniently used to add barcodes to large numbers of molecules (e.g., 1 million to 10 million to 100 million to 1 billion molecules) in one library with reduced cost as compared to strategies that require barcoded beads.

The methods disclosed herein generate a nested set of nucleic acid constructs for each genomic fragment and generate a plurality of nested sets for a plurality of genomic fragments. The nucleic acid constructs may be single-stranded or double-stranded. Each nucleic acid construct in each nested set comprises a barcode and target sequence portion, and nucleic acid constructs within each nested set have different lengths. The nucleic acid constructs in each nested set share a unique barcode sequence. The target sequence portions having a first end and second end. The nucleic acid constructs in each nested set share identical nucleotide sequences near the first ends but differ in nucleotide sequences near the second ends. The methods can sequence near both the first and second ends of all nucleic acid constructs in the nested set, and the sequence reads are assembled to produce the sequence information for the entire long genomic DNA fragment. Various approaches to achieve this objective are described below.

In some approaches, the method provides ways to retain the information that can be used to identify the position of each nucleic acid sequence (corresponding to each sequence read) in original long DNA genomic DNA molecule. This positional information is useful to decipher sequence information for long DNA molecules with repetitive sequences.

### 2. Definitions

Components or a reaction in “a single reaction mixture” means that the reaction occurs in a single mixture without compartmentalization into separate tubes, vessels, aliquots, wells, chambers, or droplets during tagging steps. Components can be added simultaneously or in any order to make the single reaction mixture.

As used herein, “a first end” and “a second end” are used to define the two ends of each nucleic acid molecule in a nest set of nucleic acid molecules. The target sequence near the first ends of the nucleic acid molecules share the same nucleotide sequence and the but differ in nucleotide sequences near the second ends. In a double-stranded DNA molecule, the first end can be either the 5-prime end or the 3-prime end. Similarly, in a double stranded DNA molecule, the second end can be either the 5-prime end or the 3-prime end. Relative to the second end in the same molecule, the first end is closer to the barcode sequence.

As used herein, “unique molecular identifier” (UMI) refers to sequences of nucleotides present in DNA molecules that may be used to distinguish individual DNA molecules from one another. See, e.g., Kivioja, Nature Methods 9, 72-74 (2012). UMIs may be sequenced along with the DNA sequences with which they are associated to identify sequencing reads that are from the same source nucleic acid. The term “UMI” is used herein to refer to both the nucleotide sequence of the UMI and the physical nucleotides, as will be apparent from context. UMIs may be random, pseudo-random, or partially random, or nonrandom nucleotide sequences that are inserted into adapters or otherwise incorporated in source nucleic acid molecules to be sequenced. In some embodiments, each UMI is expected to uniquely identify any given source DNA molecule present in a sample. For purpose of this disclosure, the term “UMI” is used interchangeably with the term “barcode.”

As used herein, the term “single tube LFR” or “stLFR” refers to the process described in, e.g., US patent publication 2014/0323316 and Wang et al., Genome Research, 29: 798-808 (2019), the entire content of each of which is hereby incorporated by reference in its entirety. In stLFR, multiple copies of the same, unique barcode sequence (or “tag”) are associated with individual long nucleic acid fragments. In one embodiment of single tube LFR, the long nucleic acid fragment is labeled with barcodes at regular intervals. In one embodiment, the barcodes are introduced into the long nucleic acid molecule using one or more enzymes, e.g., transposases, nickases, and ligases. The barcode sequences among nucleic acid fragments can be conveniently performed in, e.g., a single vessel, without compartmentalization. This process allows analysis of a large number of individual DNA fragments without the need to separate fragments into separate tubes, vessels, aliquots, wells, or droplets during tagging steps.

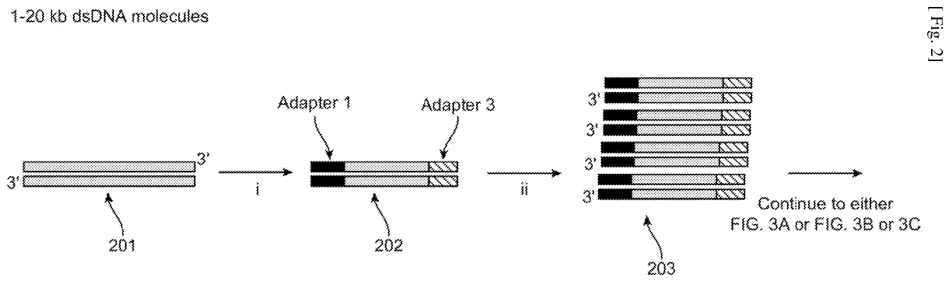

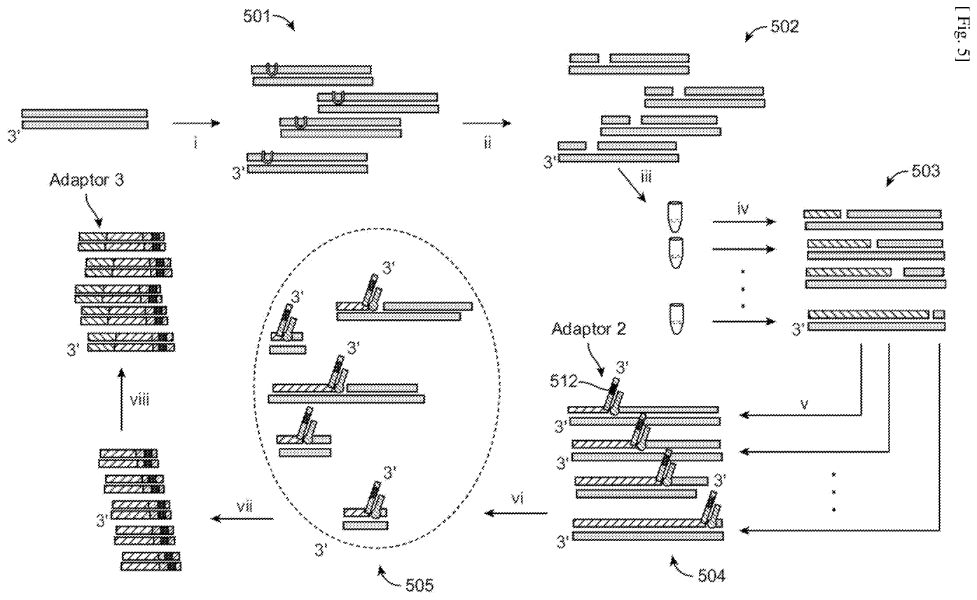

As used herein, a “unique” barcode refers to a nucleotide sequence that is used to identify an individual group of polynucleotides and distinguish it from other groups of polynucleotides among a mixture of groups. For example, a unique barcode for a nested set of nucleic acid constructs means the barcode sequence associated with one nested set is different from the barcode sequence associated with at least 90% of the total nested sets, more often at least 99% of the total nested sets, even more often at least 99.5% of the total nested sets, and most often at least 99.9% of the total nested sets. In some embodiments, a unique barcode is used to identify the position of a group of nucleic acid fragments in relation to the genomic DNA from which the group of nucleic acid fragments is derived. This barcode of this type is also referred to as positional barcode in this disclosure. In some cases, different groups of nucleic acid fragments each carrying a unique positional barcode exist in one single mixture. See, for example, [316] in FIG. 3C. In some cases, different groups of nucleic acid fragments each carrying a unique positional barcode are in separate aliquots and these separate aliquots can then be combined into one mixture. See, for example, [504] and [505] in FIG. 5.

The term “in solution,” when used in connection with an adapter (or any other nucleic acid constructs or polynucleotide complex) used in the methods or compositions disclosed herein, refers to that the adapter (or any other polynucleotide or polynucleotide complex) is not immobilized on a substrate and can freely move in solution. When used to describe a reaction, as in “a reaction performed in solution” refers to the reaction that occurred between nucleic acids, all of which are in solution.

The term “adaptered nucleic acid fragment,” and “adaptered fragment” are used interchangeably and refer to a polynucleotide comprising one target nucleic acid fragment and one or more adapter sequences.

The term “adapter sequence,” refers to a sequence on either strand of an adapter as will be clear from context. That is, “adapter sequence,” can refer to either or both the sequence of an adapter on one strand and the complementary sequence on the second strand. Likewise, the term “barcode sequence,” refers to the sequence of a barcode on one strand or its complementary sequence.

The terms “reversible terminator nucleotide,” and “reversible terminator” are used interchangeably and refer to a nucleotide having a 3-prime reversible blocking group. “Reversible blocking group” refers to a group that can be cleaved to provide a hydroxyl group at the 3′-position of the nucleotide that can be ligated to the 5-prime phosphate group of another nucleotide. The reversible blocking group can be cleavable by an enzyme, a chemical reaction, heat, and/or light. Exemplary nucleotides having 3-prime reversible blocking groups are known in the art and also disclosed in U.S. Pat. No. 10,988,501; the entire disclosure of which is herein incorporated by reference.

The term “target sequence,” refers to the sequence information of a DNA molecule, e.g., a genomic DNA fragment. Methods and compositions provided herein can be used to determine a target sequence.

The term “target sequence portion” refers a portion of the entire target sequence or a complement of the target sequence. Multiple nucleic acid fragments may comprise sequences corresponding to different portions of the same target sequence.

The term “extended primer” refers to the DNA strand produced by extending a primer annealed to a template.

The term “copy” refers to generating a complementary nucleotide strand of a template by primer extension.

The term “correspond to,” means a DNA sequence has the same or complementary sequence of another DNA sequence.

The term “near,” as used in referencing a sequence near a reference point (for example, a nucleotide sequence near a first end) refers to the nucleotide sequence within a specified length from said reference point. The specified length is typically less than 200 bases, less than 100 bases, less than 50 bases, less than 20 bases, or less than 10 bases. In some embodiments, the specified length is in a range of 1-50 bases, e.g., 1-30 bases, or 1-20 bases.

The term “exposed 5-prime,” refers to a 5-prime terminus of a DNA fragment formed after a breakage in bond between two nucleotides in an otherwise contiguous DNA strand. Likewise, the term “exposed 3-prime,” refers to a 3-prime terminus of a DNA fragment formed only after a breakage in bond between two nucleotides in an otherwise contiguous DNA strand.

The term “length suitable for sequencing,” as used herein, refers to that a DNA strand has a length that is equal to the length of a sequence read generated by MPS sequencing. This length may be dictated sequencing methods, but in general the length of a single DNA strand suitable for sequencing falls within a range of 200 bases-1.5 bases, e.g., 300-1000 bases, 300-500 bases, or 400-600 bases or 500-1000 bases, and the length of a DNA duplex suitable for sequencing fall within a range of 200-1.5 base pairs, e.g., 300-1000 base pairs, 300-500 base pairs, or 400-600 base pairs or 500-1000 base pairs.

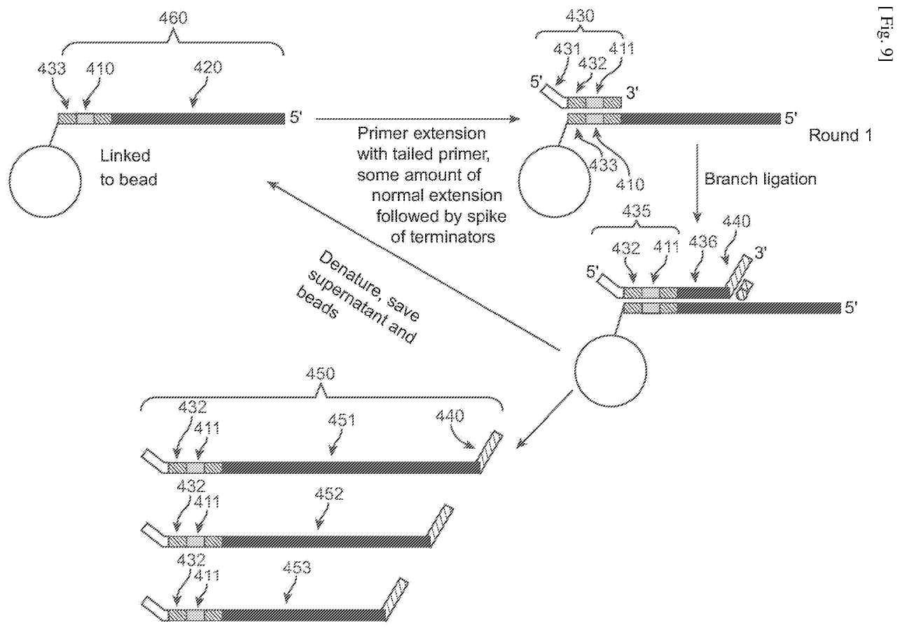

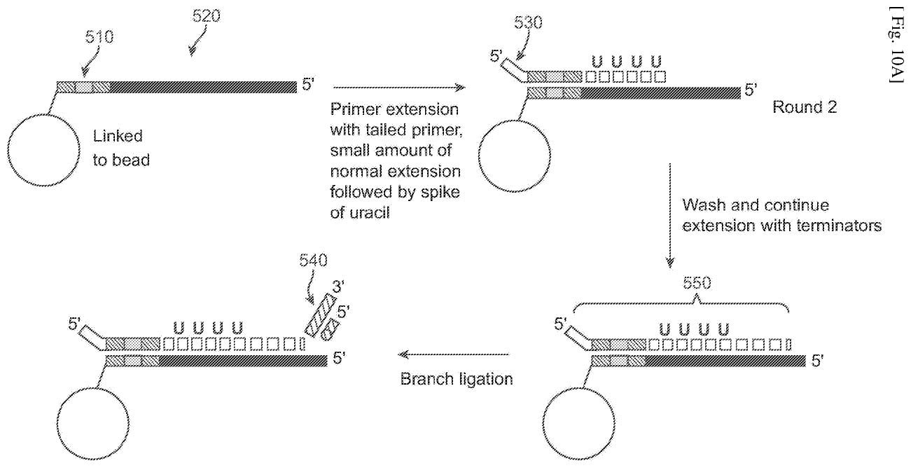

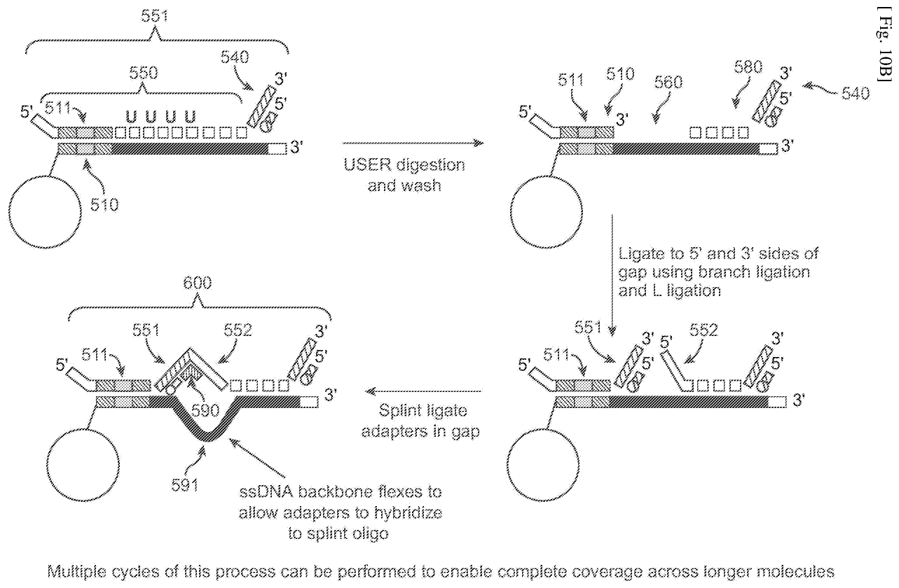

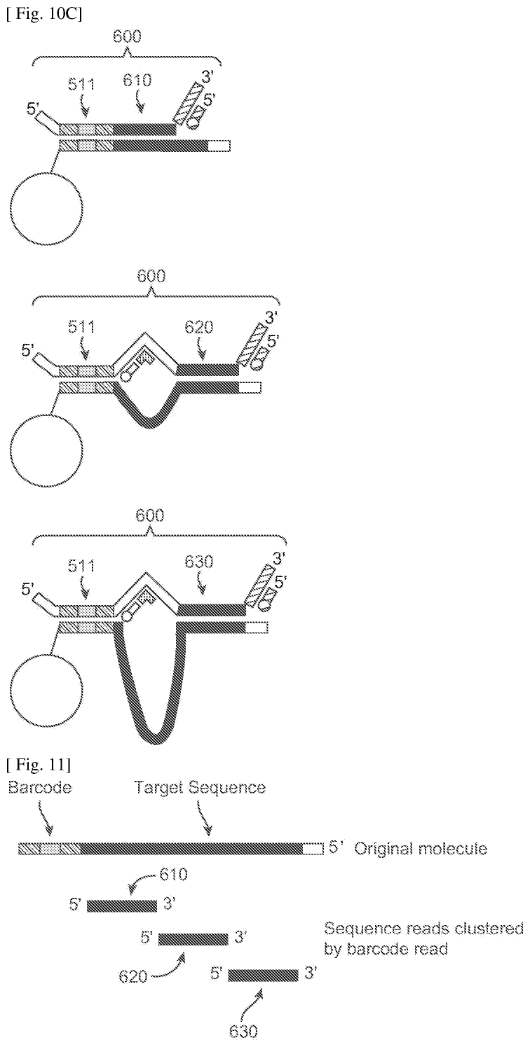

The term “join,” used in connection with a polynucleotide and a substrate (for example, a bead), refers to that the polynucleotide (or one terminus of the polynucleotide) directly contacts or is covalently linked to the substrate. For example, a surface may have reactive functionalities that react with functionalities on the polynucleotide molecules to form a covalent linkage. As one illustrative example, a barcoded fragment is joined to a bead shown in FIG. 9. The term “join,” can also be used to describe connecting one polynucleotide and another to form one single contiguous polypeptide, for example, 551 and 552 in FIG. 10B are joined to form a single contiguous adaptered fragment.

As used in this context, “fragment” is single-stranded although, as discussed above and elsewhere herein, a fragment may be hybridized to complementary strands to, for example, form a nucleic acid complex. The term “fragment” is generally used interchangeably with the term “polynucleotide.”

As used in this context, “barcode region” refers to the region in a DNA molecule where a barcode or the complement of the barcode is located.

The term “barcoded fragment,” refers to a fragment that comprises a barcode sequence or a complement of a barcode sequence.

The term “branch adapter,” refers to a partially double-stranded adapter. Said partially double-stranded adapter comprises (i) a double-stranded blunt end comprising a 5′ terminus of one strand and a 3′ terminus of the complementary strand and (ii) a single-stranded region comprising a barcode sequence. The 5′ terminus of the double-stranded region of the branch adapter can be ligated to the 3′ terminus of the nucleic acid fragment via branch ligation as further described below.

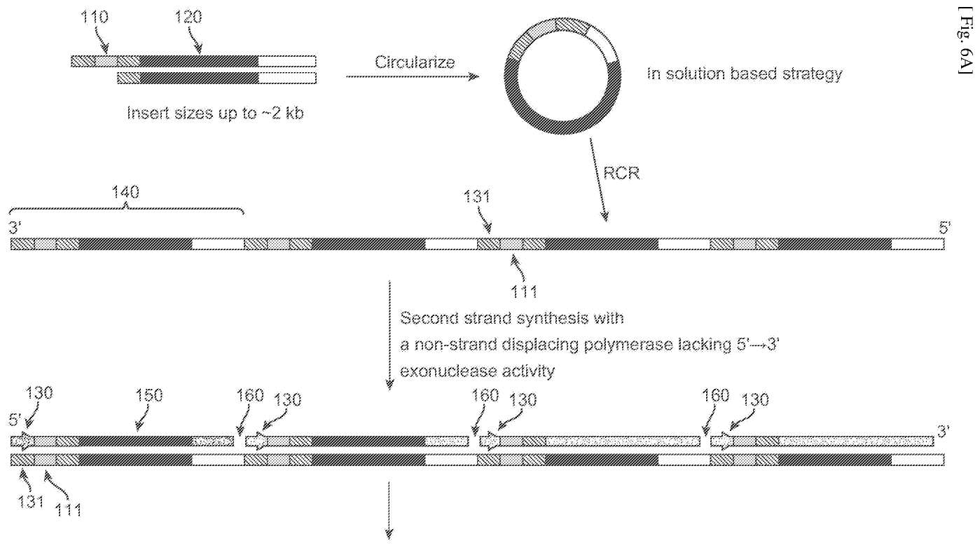

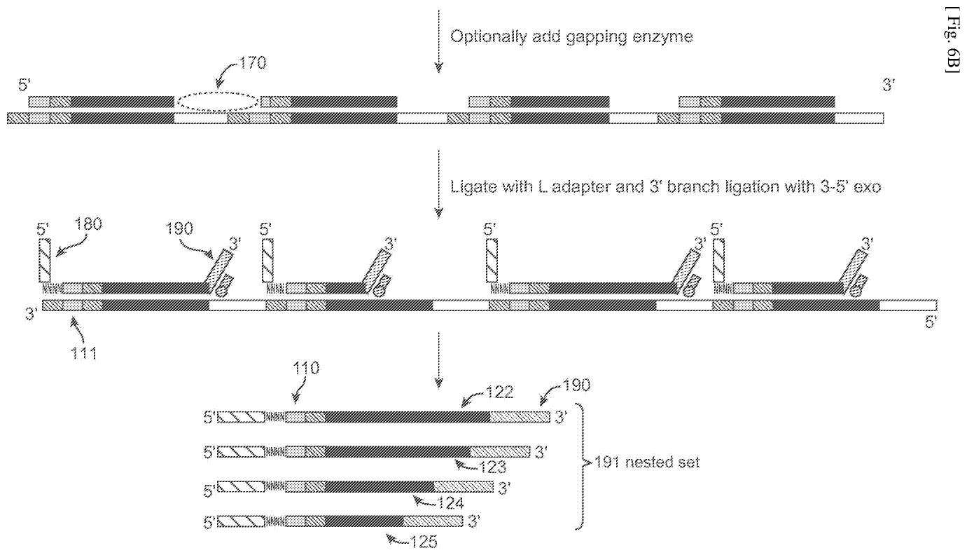

The term “nested set” refers to a plurality of nucleic acid fragments that (i) have different length, (ii) share identical nucleotide sequence at one end, and (iii) have different nucleotide sequence at the other end by truncation. One example of a nested set is shown as 191 in FIG. 6B.

The term “5-prime portion” of a polynucleotide refers to a contiguous nucleotide sequence region of the polynucleotide including the 5-prime terminus. The 5-prime portion may account for at least 10%, at least 20%, at least 30%, at least 40%, at least 50%, at least 60%, or at least 70% of the full length of the polynucleotide. The “5-prime portion” of a polynucleotide does not include the 3-prime terminus of the polynucleotide.

The term “3-prime portion” of a polynucleotide refers to a contiguous nucleotide sequence region of the polynucleotide including the 3-prime terminus. The 5-prime portion may account for at least 10%, at least 20%, at least 30%, at least 40%, at least 50%, at least 60%, or at least 70% of the full length of the polynucleotide. The “3-prime portion” of a polynucleotide does not include the 5-prime terminus of the polynucleotide.

The term “middle portion” of a polynucleotide refers to the portion between the 3-prime portion and the 5-prime portion.

The term “bubble” refers to the configuration of a DNA structure consisting of two DNA strands which comprises a non-hybridized region flanked by two double stranded regions. The non-hybridized region comprises two single-stranded loops, which lack sufficient complementarity such that they do not anneal to each other. An illustration of a bubble region is shown in FIG. 10C.

The term “interval” refers to a space separating two single-stranded nucleic acid fragments.

The term “gap” refers to an interval that has been widened (used interchangeably with the term “extended”). An interval is widened to form a gap. However, this does not necessarily mean that an interval is always smaller in length than a gap. For example, one particular interval may be larger in length than a gap formed by widening a different interval.

A process of long fragment library preparation for sequencing can be carried out according to various schemes. Described below are exemplary embodiments of the methods. A practitioner with skill in the arts of molecular biology and sequencing guided by this disclosure will recognize numerous variations of individual steps and reagents can be incorporated into the schemes below.

### 3. Methods

### 3.1 Adding Adapters to the Ends of Nucleic Acid Molecules

Various approaches can be used to add adapter sequences to one or both ends of a nucleic acid molecule, e.g., a genomic fragment. This can be done through e.g., adapter ligation, PCR amplification, and other methods that are known in the art.

In some approaches, each of a plurality of genomic fragment is ligated to an adapter comprising a barcode sequence that is unique for each genomic fragment. This unique barcode sequence can later be used to identify all reads emanating from a particular genomic fragment. Methods for labeling each genomic fragment with a unique barcode are well known and are also described further below, see the section entitled “Barcode.”

In various approaches, each genomic fragment is ligated to a first adapter at one end and a third adapter at the other end and is amplified by extending primers hybridized to the two adapters. The term “first,” “second,” or “third” are arbitrary and are used to refer to separate adapters. Unless specifically defined in context of the disclosure, they do not connote any specific physical relationship between the location where they appear in the genomic fragment, nor do they refer to any specific order in which the adapters used in the methods.

In some approaches, the first adapter comprises a barcode sequence as described above and a primer-binding sequence in a configuration such that when extending a primer that binds to the primer-binding sequence, the extension product will comprise in the order from the 5-prime to 3-prime, the primer sequence, the barcode sequence, and the target sequence.

### 3.2 Circularization

Methods of producing DNA circles are known. In one exemplary embodiment, a splint oligonucleotide of e.g., 8-40 base are annealed to both ends of the single-stranded molecules. These annealed oligos enable a 1-10 base overlap between the two ends of the product. Ligation can then be performed with T4 DNA ligase to create a single-stranded circle with a small region of double-stranded DNA at the site of ligation.

Circularization of single-stranded DNA molecules can be performed using methods well known in the art. In some approaches, for each of some single-stranded nucleic acid molecules, a splint oligo is then added, which hybridizes to the adapter sequences added to both termini of the target nucleic acid fragments. The single-stranded nucleic acids are then circularized in the presence of a ligase (e.g., T4 or Taq ligase). The DNA polymerase used for RCR can be any DNA polymerase that has strand-displacement activity, e.g., Phi29, Bst DNA polymerase, Klenow fragment of DNA polymerase I, and Deep-VentR NDA polymerase (NEB #MO258). These DNA polymerases are known to have different strengths of strand-displacement activity. It is within the ability of one of ordinary skill in the art to select one or more DNA polymerases suitable for the methods and compositions disclosed herein.

### 3.3 Aliquoting

Some approaches disclosed herein involves aliquoting a reaction mixture. Aliquots, used interchangeably with “pools,” refer to partitions of a whole. Different aliquots of the whole are similar in volume and compositions at the time the aliquots are formed. As used in this application, different aliquots may be subjected to different processing procedures and as a result they may acquire different compositions. For example, in some approaches of the disclosure, adapters having different positional barcodes are added to different aliquots, which results in aliquots with different compositions. Preferably, DNA fragments in each aliquot are of similar length. For aliquots having long and short DNA, specific methods can be used to minimize over coverage of shorter fragments. The products are PCR amplified and then split into 10-20 pools followed by controlled extension or ExoIII digestion or controlled nick translation, which proceeds for different duration of times for different pools. Short DNA fragments will be extended to completion to form blunt ends, and these fragments with blunt ends can be blocked from branch ligation using methods known in the art, for example, DNA tailing or 3′ blocking by terminal transferase. Exemplary methods of blocking short fragments from ligations are disclosed in WO2023001262, for example, section 7.2, entitled “Remove excel adapters”, the entire disclosure of said application is herein incorporated by reference in its entirety.

### 3.4 Controlled Extension

In some approaches, the method comprises extending a primer hybridized to a DNA fragment under conditions that permit controlling of the extent of an extension reaction. These extension-controlling conditions include, but are not limited to, selecting a polymerase (s) with a suitable polymerization rate or other properties, and using a variety of reaction parameters including (but not limited to) reaction temperature, duration of the extension, primer composition, DNA polymerase, primer and nucleotide concentration, additives, and buffer composition. In some cases, the extension can be controlled by a mixture of reversible terminator nucleotides and normal nucleotides for the extension. The ratio of the amount of reversible terminator nucleotides to the amount of normal nucleotides can be adjusted to achieve the desired extent of the extension. In general, a higher ratio of the amount of reversible terminator nucleotides to the amount of normal nucleotides will result in a less complete extension.

In some approaches, the amplified genomic fragments are distributed into a plurality of aliquots, and individual aliquots of the amplified genomic fragments are subject to different extension-controlling conditions, such that the extension products in different aliquots have different lengths. The individual aliquots may be in different vessels or different wells. The individual aliquots may also be in different partitions (e.g., droplets) in the same vessel.

The number of aliquots needed depend on the length of the target sequence and the length of sequence reads generated from the sequencing platform. Typically, the larger the size of the amplicon, the higher the number of aliquots are needed. In one illustrative example, for a 5 kb amplicon and 500 bases per read (pair end read length of 250 bases or single end length of 500 bases), typically 10-20 aliquots are used in the method. In some approaches, there are at least 3, at least 4, at least 5, at least 6, at least 7, at least 8, or at least 9 aliquots. In some approaches, the number of aliquots may fall in a range of 3-100, e.g., 5-50, 6-40, or 10-20.

To produce extension products in individual aliquots, a primer is annealed to the primer-binding sequence in the adaptered genomic fragment, and the primer is extended to copy the barcode sequence and beyond, i.e., extending into target sequence portion of the adaptered genomic fragment. The extension reactions in individual aliquots are controlled as discussed above, resulting in extension products having different sequences near the ends of extension products. In some approaches the extensions in different aliquots are terminated at different times for different aliquots. In some approaches, individual aliquots are extended for gradually increasing amount of time; for example, the first aliquot is extended for 2 minutes, the second aliquot is extended for 4 minutes, and so on. FIG. 3B. The length of time for the extensions in the aliquots may range from 10 seconds to 20 minutes. The extension can also be controlled by limiting the concentration of nucleotides in such a way that extension stops after 100 to 1,000 bases as a result of exhaustion of supply of nucleotides. In some approaches, the extension is performed using a polymerase with 5′ to 3′ exo activity, which may be suitable for performing nick-translation. Non-limiting examples of DNA polymerases that can be used in the methods include E. coli. DNA polymerase 1.

### 3.5 Controlled Digestion

In some approaches, the amplified genomic fragments are distributed into a plurality of aliquots and then are digested with a nuclease. In some embodiments, the nuclease is a double-stranded DNA nuclease with the 3′→5′ nuclease activity, such as ExoIII and Klenow. In some approaches, the digestions are controlled such that the length of the polynucleotide remaining after the digestion in different aliquots are different. The extent of the digestion can be controlled by parameters such as, reaction temperature, duration of the digestion, nuclease concentration, etc. In one exemplary approach, the time of digestion in individual aliquots are different such that the polynucleotides remaining after the digestion have different lengths. In one approach, the digestion of individual aliquots occurs in gradually increased time intervals such that the fragments after digestion in different aliquots have gradually decreased lengths, for example, the lengths of the fragments in different aliquots are 500 bases apart. A second adapter is then ligated to the newly formed ends after digestion via branch ligation in each aliquot, thereby producing the single-stranded nucleic acid constructs, each comprising a target sequence portion flanked by the first adapter sequence and the second adapter sequence.

### 3.6 Size Selection

In some approaches, the adaptered fragments or amplified adaptered fragments with lengths within a range that are suitable for sequencing are selected. Methods for selecting DNA fragments having desired lengths are well-known. One exemplary approach is to use AMPure XP beads, for example, the ones available from Pacific Biosciences (Menlo Park, California), part number 100-265-900, to select fragments having the desired lengths.

### 3.7 Amplification

Various methods involve amplifications, e.g., amplification of the genomic fragments or adaptered DNA fragments. Such amplification methods include without limitation: multiple displacement amplification (MDA), polymerase chain reaction (PCR), ligation chain reaction (sometimes referred to as oligonucleotide ligase amplification OLA), cycling probe technology (CPT), strand displacement assay (SDA), transcription mediated amplification (TMA), nucleic acid sequence-based amplification (NASBA), rolling circle amplification (RCR) (for circularized fragments), and invasive cleavage technology. Amplification can be performed after fragmenting or before or after any step outlined herein.

In some approaches, amplification is performed on adaptered genomic fragments by extending primers annealed to the adapter sequences. In some approaches, the genomic fragments having different target sequences are ligated to adapters at both ends, and the adapters share with common sequence. The genomic fragments are then amplified using the primers hybridized to the adapters at both ends. In some approaches, at least one of the adapters comprises a barcode.

In some approaches, the amplification is performed using target-specific primers, i.e., primers that hybridize to target sequence in the genomic DNA. In some approaches, the target-specific primers containing a common adapter tag with a random barcode to amplify specific regions.

In some approaches, the amplification can be a multi-plex PCR, i.e., using multiple primer pairs targeting different target sequences in the genomic DNA. In some approaches, the amplification is a multiplex PCR in which 2-1000 of different target regions are amplified using target-specific primers in one reaction, such that the reaction mixture comprises amplified genomic fragments having different target sequences.

In some approaches, adaptered fragments or genomic fragments can be amplified using rolling circle amplification (RCR). Genomic fragments are first denatured into single-stranded nucleic acid molecules. A splint oligo is added and hybridized to the adapter sequences flanking the target sequences, and the single-stranded nucleic acids are then circularized in the presence of a ligase (e.g., T4 or Taq ligase). The DNA polymerase used for RCR can be any DNA polymerase that has strand-displacement activity, and exemplary DNA polymerases include Phi29, Bst DNA polymerase, Klenow fragment of DNA polymerase I, and Deep-VentR NDA polymerase (NEB #MO258). These DNA polymerases are known to have different strengths of strand-displacement activity. It is within the ability of one of ordinary skill in the art to select one or more suitable DNA polymerase used for the invention.

### 3.8 Nicking

In various embodiments of the disclosure, genomic fragments or amplified genomic fragments (including those incorporating one or more adapter sequences) are combined with one or more nicking agents to create nicks in the genomic DNA fragments.

In some approaches, the nicking agent is an enzyme (generally referred to as a ‘nickase’). A nickase can be an endonuclease that cleaves a phosphodiester bond within a polynucleotide or removes one nucleotide from the polynucleotide. In some cases, the nickase is a non-sequence specific endonuclease, which nicks a DNA strand at random positions. Non-limiting examples of nicking agents include vibrio vulnificus nuclease (Vvn), Shrimp dsDNA specific endonuclease, and DNAse I. In some approaches, the nicking agent is a site- or sequence-specific nuclease, such as, a restriction endonuclease that nicks DNA at its recognition sequence. Non-limiting examples of site-specific nickases include Nt. CviPII (CCD), Nt. BspQI, and Nt. BbvCI, as described in Shuang-yong Xu, BioMol Concepts 2015; 6 (4): 253-267, the entire disclosure is herein incorporated by reference

In some approaches nicking agents disclosed herein are chemical nicking agents. Non-limiting examples of the chemical nicking agents include dipeptide seryl-histidine (Ser-His), Fe2+/H₂O₂, or Cu (II) complexes/H₂O₂.

In some approaches, the method uses two or more nicking agents. In some approaches the method used two or more nicking agents from the same category of nicking agents, e.g., any one category of non-specific nickase, site-specific nickase, or chemical nicking agents. In some approaches, the method uses nicking agents from different categories.

The length of the genomic fragments separated by the nicks after the treatment may vary. Typically, a higher the concentration of the nicking agent would produce more nicks which results in shorter fragments. A longer treatment time would similarly produce more nicks which results in shorter fragments. By adjusting one or more of these parameters, the length of the fragments can be controlled within the desired range. In some approaches, the average length of the nucleic acid fragments resulting from the nicking is between 200 and 10000 nucleotides, e.g., 200-500 nucleotides or 400-1000 nucleotides, or 1000-10000 nucleotides. One exemplary embodiment of using a nicking agent to generate nicks in the genomic fragments is shown in FIG. 3A.

In some embodiments, nicks created by the nickase are extended (widened) by an exonuclease to form gaps. This process can be referred to as “gapping,” and the exonucleases used in process can be referred to as “gapping enzymes.” Examples of enzymes with 3′ exonuclease activity include DNA Polymerase I, Klenow Fragment (in the absence of nucleotides), Exonuclease III, and others known in the art. Examples of enzymes with 5′ exonuclease activity include Bst DNA polymerase, T7 exonuclease, Exonuclease VIII truncated, Lambda exonuclease, T5 exonuclease, and other exonucleases known in the art. Low processivity exonucleases (i.e., exonucleases that remove nucleotides from the end of a polynucleotide at a relatively low rate) are preferred to open a short gap (e.g. 2-7 bases, 3-10 bases, or 3-20 bases) and disassociate from DNA to allow adapter ligation. In the case where an exonuclease is used, if necessary, protection of the DNA adapters from exonuclease digestion can be achieved by introducing phosphorothioated bonds between bases (or modified bases) at the 5′ and 3′ ends of the adapters.

Nicking and gapping of the amplified double-stranded genomic fragments produce DNA fragments having different length that end with exposed 3-prime ends, and a second adapter comprising a second adapter sequence can be ligated to the 3-prime ends via branch ligation. This process produces ligation products at least some of which are flanked by the first adapter sequences comprising the barcode sequences and the second adapter sequences. The ligation products are separated from the complementary strands they hybridize to by denaturing, thus forming nested set of single-stranded nucleic acid constructs comprising target sequence portions.

### 3.9 Nick Translation

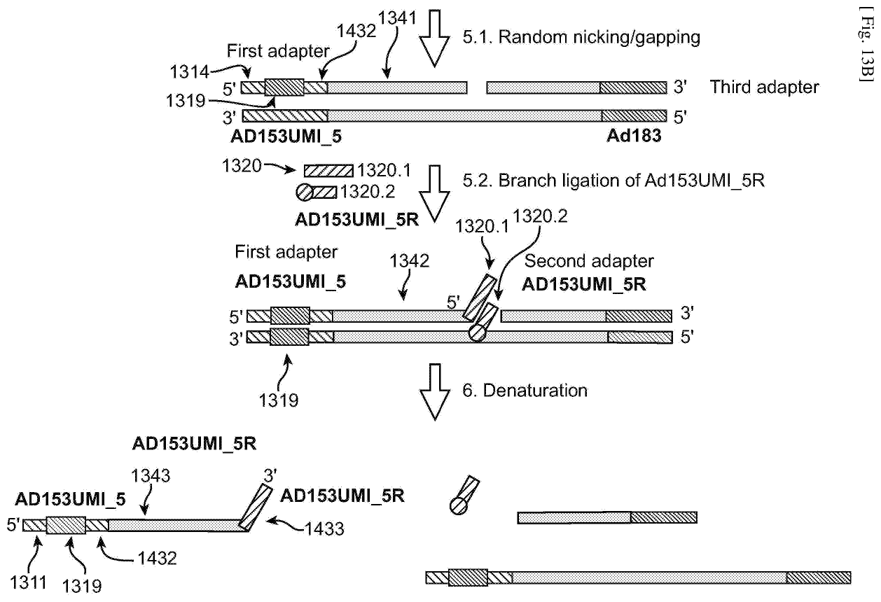

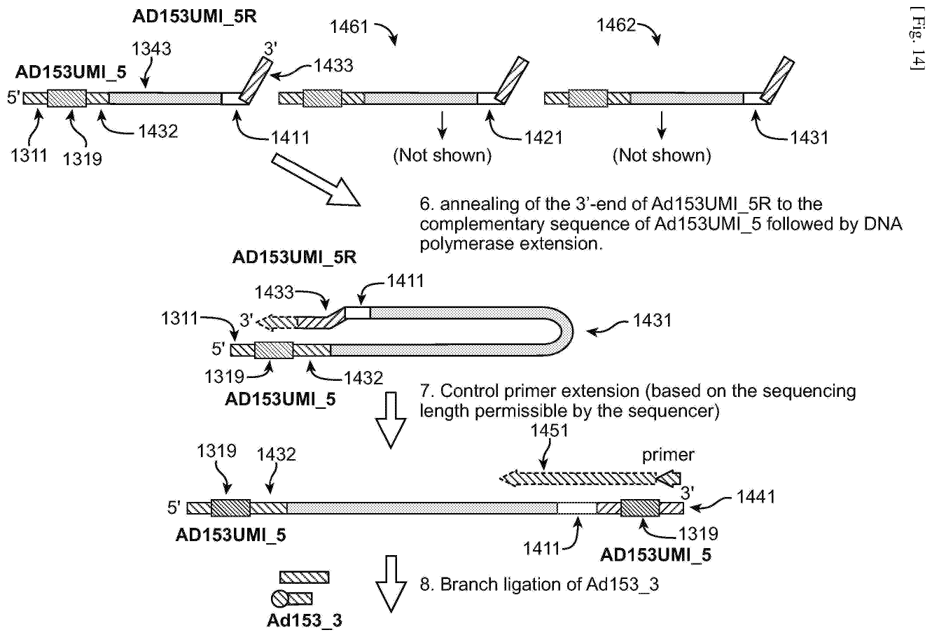

Nick translation is performed on nicks in DNA strands by a DNA polymerase (e.g., E. coli DNA polymerase I). DNA polymerases that are suitable for use in nick translation typically possess three activities: (1) a 5′ to 3′ polymerase activity that requires a single-stranded template and a primer with a 3′ hydroxyl group to synthesize a new nucleotide chain complementary to the template; (2) a 5′ to 3′ exonuclease activity that degrades double-stranded DNA from a free 5′ end; and (3) a 3′ to 5′ exonuclease activity that degrades double- or single-stranded DNA from a free 3′ hydroxyl end. This latter activity is a proofreading or editing function. On double-stranded DNA, the 3′ to 5′ exonuclease activity is blocked by the 5′ to 3′ polymerase activity. During the nick translation, the 5′ to 3′ polymerase activity of DNA polymerase adds nucleotides to the 3′-OH created by the nicking, while the 5′ to 3′ exonuclease activity simultaneously removes nucleotides from the 5′ side of the nick. The result of these concerted activities is that nucleotides are eliminated from the 5′ side of the nick while nucleotides are added to the 3′ side of the nick. This results in the movement- or translation-of the nick along the DNA. See Susan J. Karcher, Molecular Biology, A Project Approach, 1995, pages 135-192, the relevant portion is herein incorporated by reference. Nick translation may be used in various embodiments of the methods, for example, in FIG. 13A.

### 3.10 Branch Ligation

Branch ligation, also referred to as “3-prime ligation” or “3-prime branch ligation,” relies on a property of T4 ligase, ligates a double-stranded DNA adapter to a 3-prime end of DNA in an interval or gap. See, Wang et al., DNA Research, 2019 Feb. 1 16 (1):45-53, the entire disclosure is herein incorporated by reference. Branch ligation is efficient in ligating adapters because it does not require degenerate single-stranded bases on the end of the adapter to hybridize in the gap.

Adapters suitable for use in the branch ligation typically comprise: (i) a double-stranded blunt end comprising a 5-prime terminus of one strand and a 3-prime terminus of the complementary strand (ii) a single-stranded region comprising a barcode sequence. The double-stranded blunt end provides a 5-prime phosphate which can be ligated to the 3-prime of the target nucleic acid fragments via 3-prime branch ligation. In some embodiments, the double-stranded blunt end provides a 3-prime that is blocked from ligation by a dideoxynucleotide, 3′ phosphate group, 3′ overhang or the like. 3-prime branch ligation involves the covalent joining of the 5-prime phosphate from a blunt-end adapter (donor DNA) to the 3-prime hydroxyl end of a duplex DNA acceptor at 3-prime recessed strands, gaps, or intervals. In contrast to conventional DNA ligation, 3-prime branch ligation does not require complementary base pairing. 3-prime branch ligation is described in Wang et al., DNA Res. 26 (1) 45-53, doi: 10.1093/dnares/dsy037; PCT Pub. No. WO 2019/217452; US Pat. Pub. US2018/0044668 and International Application WO 2016/037418, US Pat. Pub. 2018/0044667, all incorporated by reference for all purposes.

In various embodiments, branch ligation is used to join an adapter to the genomic fragments. In some approaches nicks are introduced to the amplified genomic fragments, generating exposed 3-prime termini and 5-prime termini, then a second adapter is ligated at the nicks via branch ligation to form adaptered fragments. In some approaches, where controlled extension or digestion is performed using the genomic fragment as a template, the second adapter is then ligated at the newly formed 3-prime terminus of the extension product. The ligation thus generates adaptered fragments having the barcode sequence at the first end and the second adapter sequence at the second end.

The adapter used in the branch ligation in some cases contain additional information that are useful for the assembly of sequence reads. In some approaches, the second adapter comprises a positional barcode that is specific to individual aliquots. Fragments incorporating the second adapters in different aliquots comprise different positional barcode sequence, and fragments incorporating the second adapters in the same aliquot share the same positional barcode sequence. Aliquots in which DNA fragments now incorporating the positional barcode can be combined and sequenced. The presence of positional barcode can be used to determine long genomic fragments which have highly repetitive sequences. For example, the same sequence read from two aliquots will be assigned as duplicates in two different genomic locations rather than being erroneusly treated as one sequence read for one genomic location. The methods and compositions disclosed herein can accurately determine sequence information of highly repetitive sequences and thus useful for sequencing target sequences that are located in genomic loci where highly repetitive sequence are found, for example DNA fragments near the telomeres. The methods and compositions using these positional barcode may also valuable in sequencing target genes duplications of which correlate with a disease condition.

In some approaches, the genomic fragments were ligated with first adapters having the barcode and then ligated with second adapteres via branch ligation. In some approaches, the second adapters comprise the positional barcodes, and the branch ligation with the second adapters results in genomic fragment flanked by the first adapter sequence comprising the barcode sequence, which is unique for each genomic fragment, and the second adapter sequence comprising the positional barcode, which is unique for each aliquot. The dual barcodes allows the combining all aliquots from all nested sets in one single reaction for sequencing and thus greatly increase sequencing efficiency.

### 3.11 Sequencing

Libraries of adaptered fragments can be sequenced using methods known in the art, including for example without limitation, polymerase-based sequencing-by-synthesis (e.g., HiSeq 2500 system, Illumina, San Diego, CA), ligation-based sequencing (e.g., SOLiD 5500, Life Technologies Corporation, Carlsbad, CA), ion semiconductor sequencing (e.g., Ion PGM or Ion Proton sequencers, Life Technologies Corporation, Carlsbad, CA), zero-mode waveguides (e.g., PacBio RS sequencer, Pacific Biosciences, Menlo Park, CA), nanopore sequencing (e.g., Oxford Nanopore Technologies Ltd., Oxford, United Kingdom), pyrosequencing (e.g., 454 Life Sciences, Branford, CT), or other sequencing technologies. Some of these sequencing technologies are short-read technologies, but others produce longer reads, (e.g., the GS FLX+ (454 Life Sciences; up to 1000 bp), PacBio RS (Pacific Biosciences; approximately 1000 bp) and nanopore sequencing (Oxford Nanopore Technologies Ltd.; 100 kb). For haplotype phasing, longer reads are advantageous and require much less computation, although they tend to have a higher error rate and errors in such long reads may need to be identified and corrected according to methods set forth herein before haplotype phasing.

In some approaches, sequencing is performed using combinatorial probe-anchor ligation (cPAL) as described in, for example, US 20140051588, U.S. 20130124100, both of which are incorporated herein by reference in their entirety for all purposes.

In some approaches, sequencing is performed using DNBseq sequencers. The adaptered fragments or amplified products thereof are denatured to produce single-stranded molecules. These circles are then used to make DNA nanoballs (DNBs) for DNBseq sequencers.

In some approaches, the adaptered fragments or amplified products thereof are sequenced on Illumina or other systems that do not require circularization.

In some approaches, the sequencing is a paired-end sequencing comprising sequencing from either terminus of the same DNA fragment. In some approaches, first read reads are produced by extending a sequencing primer annealed to the adapter sequence that is closer to the first end of the target sequence fragment than the second end (“first read sequencing”), and second sequencing reads are produced by extending a sequencing primer annealed the adapter sequence that is closer the second end of the target sequence fragment than the first end (“second read sequencing”). The first read sequencing will produce the barcode sequence. The second read sequencing will produce overlapping reads to substantially or completely cover molecules up to 500 bp or 700 bp or 1000 bp in length. These overlapping sequencing reads would be clustered based on the barcode sequence determined by the first read sequencing in a de novo assembly.

In some approached, the sequencing is a single-end sequencing, and the sequence information of the genomic fragment is determined based on first read sequencing only.

### 3.12 Assemble Sequence Information

Sequence reads from the same nested set of nucleic acid constructs (derived from one genomic fragment to be sequenced, which has been ligated to an adapter having a unique barcode sequence) can be aligned based on the presence of the same barcode sequence. Sequence reads, each comprising sequence information near the first ends (which are the same) and second ends (which are variable) are assembled to provide the full length sequence of the long genomic DNA fragment.

### 4. Compositions

### 4.1 Samples

Samples containing target nucleic acids can be obtained from any suitable source. For example, the sample can be obtained or provided from any organism of interest. Such organisms include, for example, plants; animals (e.g., mammals, including humans and non-human primates); or pathogens, such as bacteria and viruses. In some cases, the sample can be or can be obtained from, cells, tissue, or polynucleotides of a population of such organisms of interest. As another example, the sample can be a microbiome or microbiota. Optionally, the sample is an environmental sample, such as a sample of water, air, or soil.

Samples from an organism of interest, or a population of such organisms of interest, can include, but are not limited to, samples of bodily fluids (including, but not limited to, blood, urine, serum, lymph, saliva, anal and vaginal secretions, perspiration and semen); cells; tissue; biopsies, research samples (e.g., products of nucleic acid amplification reactions, such as PCR amplification reactions); purified samples, such as purified genomic DNA; RNA preparations; and raw samples (bacteria, virus, genomic DNA, etc.). Methods of obtaining target polynucleotides (e.g., genomic DNA) from organisms are well known in the art.

### 4.2 Target Nucleic Acid

As used herein, the term “target nucleic acid” (or polynucleotide) or “nucleic acid of interest” refers to any nucleic acid (or polynucleotide) suitable for processing and sequencing by the methods described herein. In some approaches, the target nucleic acid is a genomic fragment, generated by fragmenting genomic DNA extracted from a sample. It is noted that while genomic fragments are used for illustration of the methods and compositions disclosed herein, sequencing libraries can also be prepared using these methods and compositions to sequence any target nucleic acid or fragments thereof, including those that contain modifications of the nucleotides, e.g., nucleotide analogs.

The nucleic acid may be single-stranded or double-stranded and may include DNA, RNA, or other known nucleic acids. The target nucleic acids may be those of any organism, including, but not limited, to viruses, bacteria, yeast, plants, fish, reptiles, amphibians, birds, and mammals (including, without limitation, mice, rats, dogs, cats, goats, sheep, cattle, horses, pigs, rabbits, monkeys and other non-human primates, and humans). A target nucleic acid may be obtained from an individual or from multiple individuals (i.e., a population). A sample from which the nucleic acid is obtained may contain nucleic acids from a mixture of cells or even organisms, such as: a human saliva sample that includes human cells and bacterial cells; a mouse xenograft that includes mouse cells and cells from a transplanted human tumor; etc. Target nucleic acids may be unamplified or they may be amplified by any suitable nucleic acid amplification method known in the art. Target nucleic acids may be purified according to methods known in the art to remove cellular and subcellular contaminants (lipids, proteins, carbohydrates, nucleic acids other than those to be sequenced, etc.), or they may be unpurified, i.e., include at least some cellular and subcellular contaminants, including without limitation intact cells that are disrupted to release their nucleic acids for processing and sequencing. Target nucleic acids can be obtained from any suitable sample using methods known in the art. Such samples include but are not limited to biosamples such as tissues, isolated cells or cell cultures, bodily fluids (including, but not limited to, blood, urine, serum, lymph, saliva, anal and vaginal secretions, perspiration and semen); and environmental samples, such as air, agricultural, water and soil samples, etc.

Target nucleic acids may be genomic DNA (e.g., from a single individual), cDNA, and/or may be complex nucleic acids, including nucleic acids from multiple individuals or genomes. Examples of complex nucleic acids include a microbiome, circulating fetal cells in the bloodstream of a expecting mother (see, e.g., Kavanagh et al., J. Chromatol. B 878: 1905-1911, 2010), circulating tumor cells (CTC) from the bloodstream of a cancer patient. In one embodiment, such a complex nucleic acid has a complete sequence comprising at least one gigabase (Gb) (adiploid human genome comprises approximately 6 Gb of sequence).

In some cases, target nucleic acids are genomic fragments. In some approaches the genomic fragments are longer than 10 kb, e.g., 10-100 kb, 10-500 kb, 20-300 kb, 50-200 kb, 100-400 kb, or longer than 500 kb. In some cases, target nucleic acids are 5,000 to 100,000 Kb. In some approaches, the target nucleic acids are 500 bases to 50,000 bases in length, e.g., 1000 bases to 20,000 bases, or 5000 bases to 10,000 bases. The amount of DNA (e.g., human genomic DNA) used in a single mixture may be <10 ng, <3 ng, <1 ng, <0.3 ng, or <0.1 ng of DNA. In some approaches, the amount of DNA used in the single mixture may be less than 3,000×, e.g., less than 900×, less than 300×, less than 100×, or less than 30× of haploid DNA amount. In some approaches, the amount of DNA used in the single mixture may be at least 1× of haploid DNA, e.g., at least 2× or at least 10× haploid DNA amount.

Target nucleic acids may be isolated using conventional techniques, for example as disclosed in Sambrook and Russell, Molecular Cloning: A Laboratory Manual, cited supra. In some cases, particularly if small amounts of the nucleic acids are employed in a particular step, it is advantageous to provide carrier DNA, e.g., unrelated circular synthetic double-stranded DNA, to be mixed and used with the sample nucleic acids whenever only small amounts of sample nucleic acids are available, and there is danger of losses through nonspecific binding, e.g., to container walls and the like.

According to some embodiments of the invention, genomic DNA or other complex target nucleic acids are obtained from an individual cell or small number of cells with or without purification by any known method.

As described above, methods of the disclosure are useful for sequencing long nucleic acid fragments. Long fragments of genomic DNA can be isolated from a cell by any known method. A protocol for isolation of long genomic DNA fragments from human cells is described, for example, in Peters et al., Nature 487: 190-195 (2012). In one embodiment, cells are lysed and the intact nuclei are pelleted with a gentle centrifugation step. The genomic DNA is then released through proteinase K and RNase digestion for several hours. The material can be treated to lower the concentration of remaining cellular waste, e.g., by dialysis for a period of time (i.e., from 2-16 hours) and/or dilution. Since such methods need not employ many disruptive processes (such as ethanol precipitation, centrifugation, and vortexing), the genomic nucleic acid remains largely intact, yielding a majority of fragments that have lengths in excess of 150 kilobases. In some approaches, the fragments are from about 5 to about 750 kilobases in length. In further embodiments, the fragments are from about 150 to about 600, about 200 to about 500, about 250 to about 400, and about 300 to about 350 kilobases in length. The smallest fragment that can be used for haplotyping is approximately 2-5 kb; there is no maximum theoretical size, although fragment length can be limited by shearing resulting from manipulation of the starting nucleic acid preparation.

In other embodiments, long DNA fragments are isolated and manipulated in a manner that minimizes shearing or absorption of the DNA to a vessel, including, for example, isolating cells in agarose in agarose gel plugs, or oil, or using specially coated tubes and plates.

According to another embodiment, in order to obtain uniform genome coverage in the case of samples with a small number of cells (e.g., 1, 2, 3, 4, 5, 10, 10, 15, 20, 30, 40, 50 or 100 cells from a microbiopsy or circulating tumor or fetal cells, for example), all long fragments obtained from the cells are barcoded using methods disclosed herein.

### 4.3 Barcode

According to one embodiment, a barcode-containing sequence is used that has two, three, or more segments of which, one, for example, is the barcode sequence. For example, an introduced sequence may include one or more regions of known sequence and one or more regions of degenerate sequence that serves as the barcode (s) or tag (s). The known sequence (B) may include, for example, PCR primer binding sites, transposon ends, restriction endonuclease recognition sequences (e.g., sites for rare cutters, e.g., Not I, Sac II, Mlu I, BssH II, etc.), or other sequences. The degenerate sequence (N) that serves as the tag is long enough to provide a population of different-sequence tags that is equal to or, preferably, greater than the number of fragments of a target nucleic acid to be analyzed. The higher the N value, the less likely two molecules will share the same barcode.

According to one embodiment, the barcode-containing sequence comprises one region of known sequence of any selected length. According to another embodiment the barcode-containing sequence comprises two regions of known sequence of a selected length that flank a region of degenerate sequence of a selected length, i.e., Bₙ Nₙ Bₙ, where N may have any length sufficient for tagging long fragments of a target nucleic acid, including, without limitation, N=10, 11, 12, 13, 14, 15, 16, 17, 18, 19 or 20, and B may have any length that accommodates desired sequences such as transposon ends, primer binding sites, etc. For example, such an embodiment may be B₂₀N₁₅B₂₀.

In one embodiment, a two or three-segment design is utilized for the barcodes used to tag long fragments. This design allows for a wider range of possible barcodes by allowing combinatorial barcode segments to be generated by ligating different barcode segments together to form the full barcode segment or by using a segment as a reagent in oligonucleotide synthesis. This combinatorial design provides a larger repertoire of possible barcodes while reducing the number of full-size barcodes that need to be generated. In further embodiments, unique identification of each long fragment is achieved with 8-12 base pair (or longer) barcodes.

In one embodiment, two different barcode segments are used. A and B segments are easily modified to each contain a different half-barcode sequence to yield thousands of combinations. In a further embodiment, the barcode sequences are incorporated on the same adapter. This can be achieved by breaking the B adapter into two parts, each with a half barcode sequence separated by a common overlapping sequence used for ligation. The two tag components have 4-6 bases each. An 8-base (2×4 bases) tag set is capable of uniquely tagging 65,000 sequences. Both 2×5 base and 2×6 base tags may include use of degenerate bases (i.e., “wild-cards”) to achieve optimal decoding efficiency.

In further embodiments, unique identification of each sequence is achieved with 8-12 base pair error correcting barcodes. Barcodes may have a length, for illustration and not limitation, of from 5-20 informative bases, usually 8-16 informative bases.

### 4.4 UMI

In various embodiments, unique molecular identifiers (UMIs) are used to distinguish individual DNA molecules from one another. The collection of adapters is generated, each having a UMI. Those adapters are attached to fragments or other source DNA molecules to be sequenced, and the individual sequenced molecules each has a UMI that helps distinguish it from all other fragments. In such implementations, a very large number of different UMIs (e.g., many thousands to millions) may be used to uniquely identify DNA fragments in a sample. One exemplary embodiment of the method using UMI is described in Example 2.

The UMI is at a length that is sufficient to ensure the uniqueness of each and every source DNA molecule. In some approaches, the unique molecular identifier is about 3-12 nucleotides in length, or 3-5 nucleotides in length. In some cases, each unique molecular identifier is about 3-12 nucleotides in length, or 3-5 nucleotides in length. Thus, a unique molecular identifier can be 3, 4, 5, 6, 7, 8, 9, 10, 11, 12, 13, 14, 15, 16, 17, 18, or more nucleotides in length.

A process of long fragment library preparation for sequencing can be carried out according to various schemes. These schemes can be used to generate a nested set of nucleic acid constructs for each genomic fragment and enable sequence determination near the two ends of each nucleic acid construct in each nested set. The nucleic acid constructs in each nested set can be either single-stranded or double-strand. These approaches allow efficient generation of sequence information for long genomic fragments. Some of these approaches involve making DNA circles. Other approaches use linear DNA molecules. Described below are exemplary embodiments of the methods. A practitioner with skill in the arts of molecular biology and sequencing guided by this disclosure will recognize numerous variations of individual steps and reagents that can be incorporated into the schemes below.

### 4.5 Barcoded Beads

The beads are barcoded by the barcode oligonucleotides in the adapters immobilized thereon. Each bead comprises multiple adapters and thus multiple barcode oligonucleotides. Each barcode oligonucleotide comprises at least one barcode. The barcode oligonucleotides on the same bead share the same barcode sequence and barcode oligonucleotides on different beads have different barcode sequences. As such, each bead carries many copies of a unique barcode sequence, which can be transferred to the target nucleic acid fragments using methods as described above.

The beads used may have a diameter in the range of 1-20 μm, alternatively 2-8 μm, 3-6 μm or 1-3 μm (e.g., about 2.8 μm). For example, the spacing of barcoded oligonucleotides on the beads is can at least 1, at least 2, at least 3, at least 4, at least 5, at least 6 or at least 7 nm. In come embodiments the spacing is less than 10 nm (e.g., 5-10 nm), less than 15 nm, less than 20 nm, less than 30 nm, less than 40 nm, or less than 50 nm. In some embodiments, the number of different barcodes used per mixture may be >1M, >10M, >30M, >100M, >300M, or >1B. As discussed below, a very large number of barcodes may be produced for use in the invention, e.g., using methods described herein. In some embodiments, the number of different barcodes are used per mixture may be >1M, >10M, >30M, >100M, >300M, or >1B and they are sampled from a pool of at least 10-fold greater diversity (e.g., from >10M, >0.1B, 0.3B, >0.5B, >1B, >3B, >1 GB different barcodes on beads.) In some embodiments, the number of barcodes per bead is between 100 k to 10M (e.g., between 200 k and 1M, between 300 k and 800 k, or about 400 k).

In some embodiments, the barcode region is about 3-15 nucleotides in length, e.g., 5-12, 8-12, or 10 nucleotides in length. In some cases, each barcode of the barcode region is about 3-12 nucleotides in length, or 3-5 nucleotides in length. Thus, a barcode, whether sample barcode, cell barcode or other barcode can be 3, 4, 5, 6, 7, 8, 9, 10, 11, 12, 13, 14, 15 or more nucleotides in length. In one example, each barcode region comprises three barcodes, each consisting of 10 bases, and the three barcodes are separated by 6 bases of common sequence.

Barcodes beads are transferred to the target nucleic acid sequence. In some embodiments, the transfer occurred at regular intervals through ligation of the 3′ terminus of the adapter oligonucleotide to the nucleic acid fragments created by nicking and the gapping as disclosed.

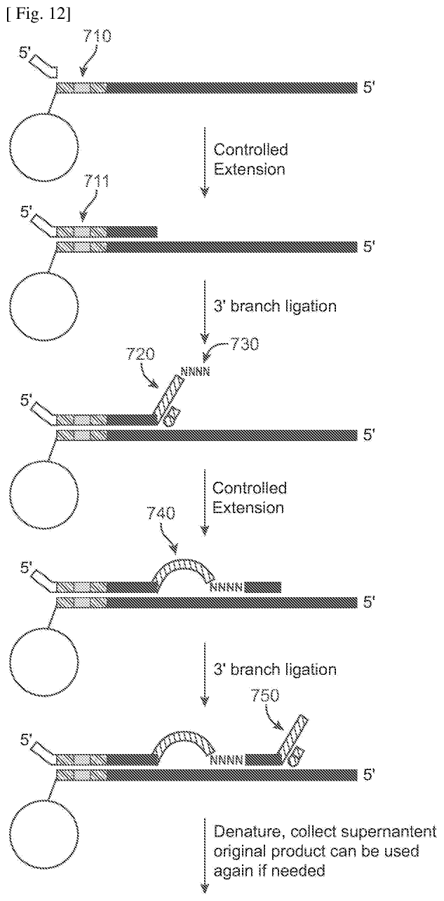

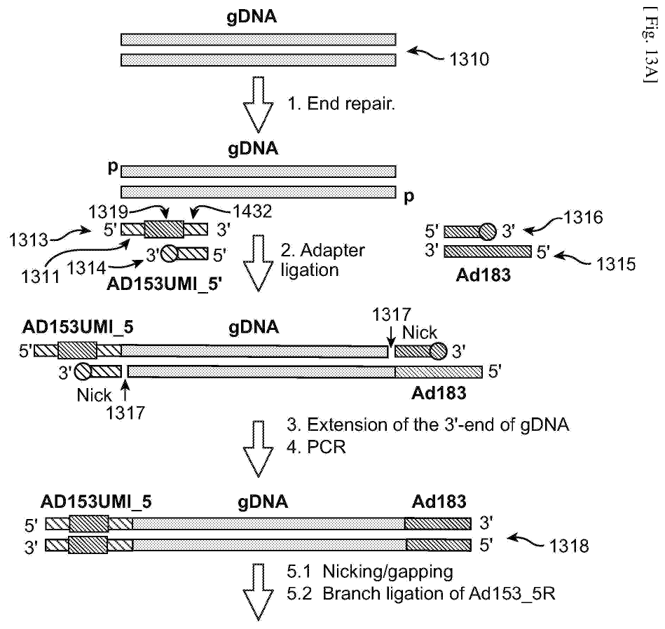

In some embodiments, the barcoded beads are constructed through a split and pool ligation-based strategy using three sets of double-stranded barcode DNA molecules. In some embodiments, each set of double-stranded barcode DNA molecules consists of 10 base pairs and the three sets are different in nucleic acid sequence. An exemplary method of the split and pool ligation to produce the barcoded beads is described in the PCT Pub. No. WO 2019/217452, the disclosure of which is herein incorporated by reference in its entirety. FIGS. 12 and 13 of WO 2019/217452 also illustrate the methodology of the split and pool method. In one approach, a common adapter sequence comprising a PCR primer annealing site was attached to Dynabeads™ M-280 Streptavidin (ThermoFisher, Waltham, MA) magnetic beads with a 5′ dual-biotin linker. Three sets 1, 536 of barcode oligos containing regions of overlapping sequence were constructed by Integrated DNA Technologies (Coralville, IA). Ligations were performed in 384 well plates in a 15 μL reaction containing 50 mM Tris-HCl (pH 7.5), 10 mM MgCl₂, 1 mM ATP, 2.5% PEG-8000, 571 units T4 ligase, 580 pmol of barcode oligo, and 65 million M-280 beads. Ligation reactions were incubated for 1 hour at room temperature on a rotator. Between ligations beads were pooled into a single vessel through centrifugation, collected to the side of the vessel using magnet, and washed once with high salt wash buffer (50 mM Tris-HCl (pH 7.5), 500 mM NaCl, 0.1 mM EDTA, and 0.05% Tween 20) and twice with low salt wash buffer (50 mM Tris-HCl (pH 7.5), 150 mM NaCl, and 0.05% Tween 20). Beads were re-suspended in 1× ligation buffer and distributed across 384 wells plates and the ligation steps were repeated.

In one aspect, the invention provides a composition comprising beads with adapter oligonucleotides comprising clonal barcodes attached, where the composition comprises more than 3 billion different barcodes and where the barcodes are tripartate barcodes with the structure 5′-CS1-BC1-CS2-BC2-CS3-BC3-CS4. In some embodiments, CS1 and CS4 are loner than CS2 and CS3. In some embodiments, CS2 and CS3 are 4-20 bases, CS1 and CS4 are 5 or 10 to 40 bases (e.g., 20-30), and the BC sequences are 4-20 bases (e.g., 10 bases) in length. In some embodiments, CS4 is complementary to a splint oligonucleotide. In some embodiments, the composition comprises bridge oligonucleotides. In some embodiments, the composition comprises bridge oligonucleotides, beads comprising a tripartate barcode as discussed above, and genomic DNA comprising hybridization sequences with a region complementary to the bridge oligonucleotides.

Another source of clonal barcodes such as a bead or other support associated with multiple copies of tags can be prepared by emulsion PCR or CPG (controlled-pore glass) or chemical synthesis other particles with copies of an adapted-barcode prepared by. A population of tag-containing DNA sequences can be PCR amplified on beads in an water-in-oil (w/o) emulsion by known methods. See, e.g., Tawfik and Griffiths Nature Biotechnology 16: 652-656 (1998); Dressman et al., Proc. Natl. Acad. Sci. USA 100: 8817-8820, 2003; and Shendure et al., Science 309: 1728-1732 (2005). This results in many copies of each single tag-containing sequence on each bead.

Another method for making a source of clonal barcodes is by oligonucleotide synthesis on micro-beads or CPG in a “mix and divide” combinatorial process. Using this process one can create a set of beads each having population of copies of a barcode. For example, to make all B₂₀N₁₅B₂₀ where each of about 1 billion is represented in ˜1000+ copies on each of 100 beads, on average, one can start with ˜100 billion beads, synthesize B₂₀ common sequence (adapter) on all of them and then split them in 1024 synthesis columns to make a different 5-mer in each, then mix them and split them again in 1024 columns and make additional 5-mer, and then repeat that once again to complete N15, and then mix them and in one big column synthesize the last B₂₀ as a second adapter. Thus, in 3050 syntheses one can make the same “clonal-like” sets of barcodes as in one big emulation PCR reaction with ˜1000 billion beads (1¹² beads) because only 1 in 10 beads will have a starting template (the other 9 would have none) to prevent having two templates with different barcode per bead.

An exemplary process for the barcode sequence assembly is described in PCT Pub. No. WO 2019/217452, and the disclosure of which is herein incorporated by reference.

### 4.6 Reaction Mixture

Provided herein is a reaction mixture useful for preparing a library of polynucleotides. The reaction mixture comprises 1) a polymerase that lacks 5-3′ exo activity and does not possess strand-displacement activity; 2) a DNA complex comprising a plurality of fragments hybridized to the one or more monomers of the DNA concatemer and separated by nicks or gaps. In some embodiments, some or all of the fragments are produced by extending RNA primers, thus these fragments incorporate RNA sequences at the 5-prime end. In some embodiments, the reaction mixture further comprises one or more gapping enzymes as described herein. In some embodiments, the gapping enzyme has 5′→3′ exonuclease activity. In some embodiments, the gapping enzyme has 3′→5′ exonuclease activity.

In some embodiments, each of the fragments is ligated to an L-adapter at the 5-prime terminus and a branch adapter at the 3-prime terminus.

Also disclosed herein is a DNA complex comprising a barcoded fragment immobilized on a solid support (e.g., a bead) and a fragment hybridized to the barcoded fragment. In some embodiments, the fragment comprises a plurality of uracils. In some embodiments, the fragment comprises a 5-prime portion, a 3-prime portion and a middle portion is located therebetween, and the middle portion of the fragment is not hybridized to the barcoded fragment. In some embodiments, the 5-prime portion of the fragment is an adapter sequence, and the 3-prime portion of the fragment comprises a branch adapter sequence. One illustrative embodiment is shown in FIG. 10A.

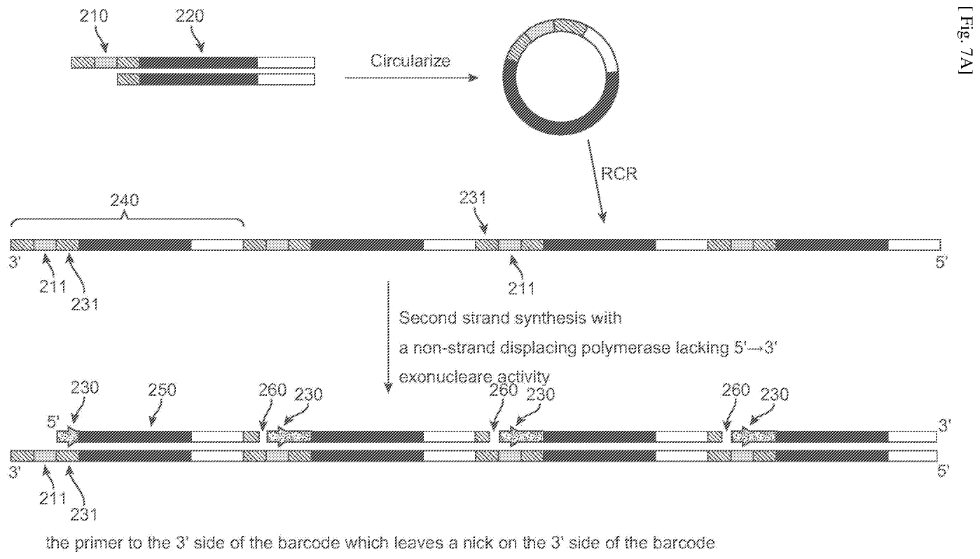

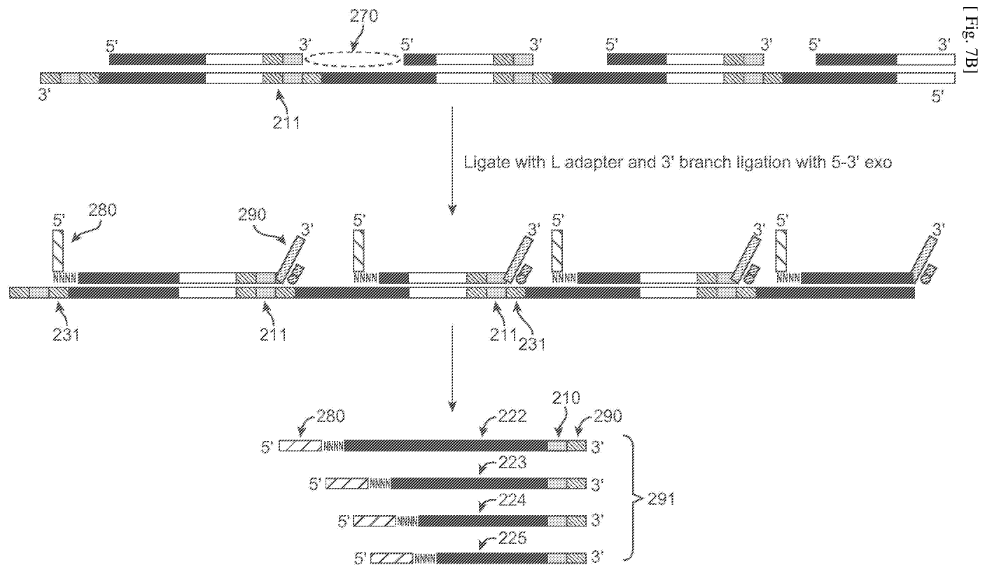

Also provided herein is a composition comprising a group of DNA fragments having overlapping target sequences. In some embodiments, the fragments having different lengths of target sequences but share the same barcode sequence. In some embodiments the fragment share a common adapter sequence in the 5-prime terminus and a common adapter sequence in the 3-prime terminus. In some embodiments, the fragments in the series share a common sequence in the 5-prime portion, which comprises the barcode sequence. One illustrative embodiment is show in FIG. 6B In some embodiments, the fragments in the series share a common sequence in the 3-prime portion, which comprises the barcode sequence. One illustrative embodiment is show in FIG. 7B.

Also provided herein is a composition comprising a plurality of nested sets of single stranded DNA loops, wherein each loop comprises a target sequence portion flanked by a first adapter sequence and a second adapter sequence. The first adapter sequence comprises, from 5′ to 3′, a primer-binding sequence, a barcode sequence and a first hybridization sequence, and the second adapter sequence comprises a second hybridization sequence. The first and the second hybridization sequences are hybridized to each other, thereby forming a loop. Each target sequence portion has a first end and a second end, wherein the distance between the first end and the barcode sequence is shorter than the distance between the second end and the barcode sequence. The single-stranded nucleic acid constructs in each nested set share the same barcode sequence and the single-stranded nucleic acid constructs in different nested sets have different barcode sequences. For each nested set of single-stranded nucleic acid constructs, the target sequence portions in that nested set have identical nucleotide sequences near the first ends and differ from each other by truncations near the second ends, such that the nested set of single-stranded nucleic acid DNA loops comprises a plurality of target sequence portions having different lengths.

### 5. DNA Circle-Based Scheme

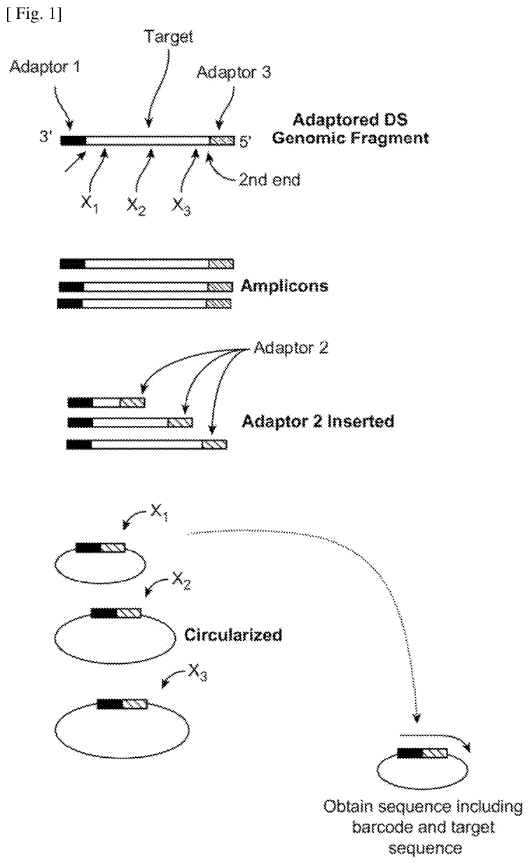

In some approaches, the methods use a DNA circle-based approach. The methods comprise circularizing the nucleic acid constructs in each nested set so that the two ends in each nucleic acid construct are joined together. See FIG. 1. Each nucleic acid construct comprises a target sequence flanked by a first adapter (“Adapter 1”) sequence and a third adapter (“Adapter 3”) sequence. Circularization of the nucleic acid constructs allows the sequences near both ends to be included in a single sequence read. The target sequence portions from different nucleic acid constructs in the same nested set can be assembled to generate sequence information that corresponds to the entire target sequence in the genomic fragment. The scheme is further illustrated below in detail.

### 5.1 Ligating Adapters to Both Ends of Genomic Fragments and Amplifying Genomic Fragments

One examplary approach of adding adapters to both ends of genomic fragments [201] is illustrated in FIG. 2. Step (i) shows ligating double-stranded genomic fragments to two double-stranded adapters at both ends to produce adaptered genomic fragments [202]. Step (ii) shows amplifying the adaptered double-stranded genomic fragments. Amplification can be performed using primers hybridizing primers to the first and third adapter sequences (not shown). This step results in amplified genomic fragments [203] with blunt ends.

### 5.2 Producing Nested Sets of Single-Stranded Nucleic Acid Constructs Comprising Target Sequences and Circularization

In the circularization approach, the amplified genomic fragments are processed to produce nested sets of single-stranded nucleic acid constructs (for example, [303] in FIG. 3A), and the single-stranded nucleic acid constructs are circularized. Each single-stranded DNA construct comprises a target sequence portion that has a first end and a second end. The target sequence portion is flanked by the first adapter sequence abutting the first end. A second adapter sequence (Adapter 2) is inserted adjacent to the second end. In some approaches, the first adapter sequence is at the 5-prime of the single-stranded DNA construct, and the second adapter sequence is at the 3-prime. The first adapter sequence comprises a barcode sequence that is unique to individual nested sets, i.e., single-stranded DNA constructs within the same nested set share the same barcode sequence and single-stranded DNA constructs from different nested sets have different barcode sequences. In some cases, the first end of the single-stranded DNA construct is closer to the barcode sequence than the second end.

Various approaches can be used to produce single-stranded nucleic acid constructs from the amplified genomic fragments. In some approaches, a nested set of single-stranded DNA constructs are generated by contacting amplified genomic fragments with nicking agents to introduce nicks in a target sequence. Then second adapters are ligated at the nicks via branch ligation. One example of such an approach is illustrated in FIG. 3A. Step (iii) of FIG. 3A shows contacting the amplified genomic fragments (generated from (ii) from FIG. 2) with a nicking agent to produce nicks at random positions in the target sequences in the amplified genomic fragments. Each amplified genomic fragment may be nicked one or more times, and the nicks produced in the fragment can be extended by using one or more exonucleases to form gaps. This process results in one or more fragments, but only one of the fragments contains the first adapter sequence (comprising the barcode sequence [311]). A number of parameters can affect the length of the nucleic acid fragments separated by the nicks and/or gaps. Typically, the higher the concentration of the nicking agent, the longer treatment time by the nicking agents, the shorter the length the fragments. By adjusting one or more of these parameters, the length of the fragments can be controlled within a desired range. In some embodiments, the average length of the nucleic acid fragments resulted from the nicking is between 200 and 10000 nucleotides, e.g., 200-500 nucleotides or 400-1000 nucleotides or 1000-10000 nucleotides. Step (iv) shows ligating a second adapter comprising a second adapter sequence at the nicks via branch ligation to form ligated products [302], each of some of the ligated products comprises a first adapter sequence and a second adapter sequence. Step (v) shows denaturing the ligated products to form single-stranded nucleic acid constructs [303], each of some ligated products comprising the first adapter sequence and the second adapter sequence. The population of single-stranded nucleic acid constructs represent a nested set of constructs comprising target sequence.

In some other approaches, generation of a nested set of single-stranded DNA constructs involves annealing a primer to the primer binding sequence in the first adapter that has been ligated to the genomic fragment and extending the primer to produce a primer extension product. One example of such an approach is shown in FIG. 3B. Step (iii) of FIG. 3B shows distributing the amplified adaptered double-stranded genomic fragments [203] as shown in FIG. 2 into a plurality of aliquots. Step (iv) shows denaturing the amplification products in each aliquot to form single-stranded molecules [304] and then hybridizing a primer to the primer-binding sequence. Said primer is extended in the presence of polymerase and dNTPs, and the extension is controlled such that the extension products in different aliquots have different lengths, thereby forming a nested set of extension products. The extension products each has a first end (where the primer starts) and a second end (where the extension ends), and the extension products share the same sequence near the first ends and have different sequences near the second ends. Step (v) shows adding second adapters to the second ends by branch ligation. In some embodiments, these second adapters comprise positional barcode sequences [312] that are unique to individual aliquots. As a result, single-stranded nucleic acid constructs [305] formed as the result of the branch ligations in different aliquots comprise different positional barcode sequences. The single-stranded nucleic acid constructs in the same aliquot share the same positional barcode sequence. The aliquots are then combined into one single mixture [306]. The dashed oval represents one single mixture. Subsequent steps are all performed in one single mixture. Step (vi) shows denaturing the product in the single mixture to form adaptered fragments [307].

In some other approaches, generation of a nested set of single-stranded DNA constructs involves ligating adapters via branch ligation having positional barcode sequences after each specified period of time. One exemplary approach is shown in FIG. 3C. Unlike the approach illustrated in FIG. 3B, the reactions in FIG. 3C are performed in single tube throughout the entire procedure-no aliquoting is needed. Step iii shows ligating a second adapter to the extended primers after the primer is extended for a first time period, resulting in a reaction mixture of fragments ligated with the second adapter [314]; step iv shows ligating an additional adapter to the extended primers (in the same reaction mixture) after the primer is extended for an additional time period, resulting in a mixture of fragments ligated with the second adapter [314] and fragments ligated with a first additional adapter [315]; step v shows adding a second additional adapter after the primer is extended for yet another period of time, which resulting a mixture of fragments ligated with the second, first additional, and second additional adapters[314], [315], and [316], and so on. The process of adding adapters with unique positional barcodes can be repeated for 3-50 rounds, e.g., 10-40 rounds, or 10-20 rounds. Each of the second, first additional, second additional adapter, and further additional adapters comprises a unique positional barcode sequence and ligation of each of these adapters to the extended primer is via branch ligation.

The molar amount of each of the adapters used in the branch ligation (as illustrated in steps (iii)-(v) of FIG. 3C is a small percentage of the total molar amount of the amplified genomic fragments, such that only a fraction of the extended primers that are available for branch ligation in each round are ligated with the adapter. In some embodiments, the molar amount of the adapter used is 1-20%, e.g., 1-10%, or 2-15% of the total molar amount of the amplified genomic fragments. In some embodiments, the amounts of the adapters (e.g., the second adapter, the first additional adapter, the second additional adapter) used in different rounds are same. In some embodiments, the amounts of the adapters used in different rounds are different. Step (vi) of FIG. 3C shows denaturing the reaction mixture [316] to produce single-stranded adaptered fragments [317]. The single-stranded fragments are then circularized as described below.

### 5.3 Circularization

The single-stranded nucleic acid constructs are then circularized to form single-stranded circles. Methods for circularization of single-stranded nucleic acids are well known, see Section 3.2. At least some of these single-stranded DNA fragments comprise the barcode sequence and a target sequence portion. In each nested set, target sequence portions share the same nucleotide sequence near the first ends but have different nucleotide sequences near the second ends. Upon circularization, the first adapter sequence and the second adapter sequence in each single-stranded nucleic acid construct are joined, which brings the first end and a second end of the target sequence portion into proximity with each other such that a single sequence read can identify the sequence information near both ends. Exemplary approaches are illustrated in FIG. 3A, step (vi) [308] and FIG. 3B, step (vii) [306].

5.4 Producing Linear Double-Stranded Adaptered Constructs from Single-Stranded Circles

Various approaches can be used to produce linear double-stranded adaptered constructs from single-stranded DNA circles (for example those generated using methods in FIG. 3A or FIG. 3B or FIG. 3C). Some of these approaches involves random fragmentation of the single-stranded circles to produce fragments and generating double-stranded adaptered constructs from these fragments. DNA circles can be fragmented using methods that are known in the art, for example, sonification, to produce a plurality of single-stranded DNA fragments. Each single-stranded DNA fragment comprises the barcode sequence that was in the DNA circle. Optionally, size selection is performed to select fragments having lengths that are suitable for sequencing.

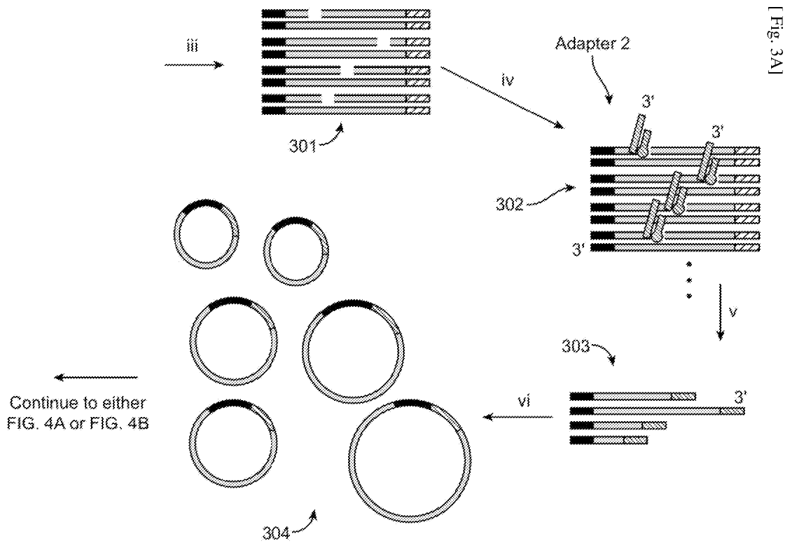

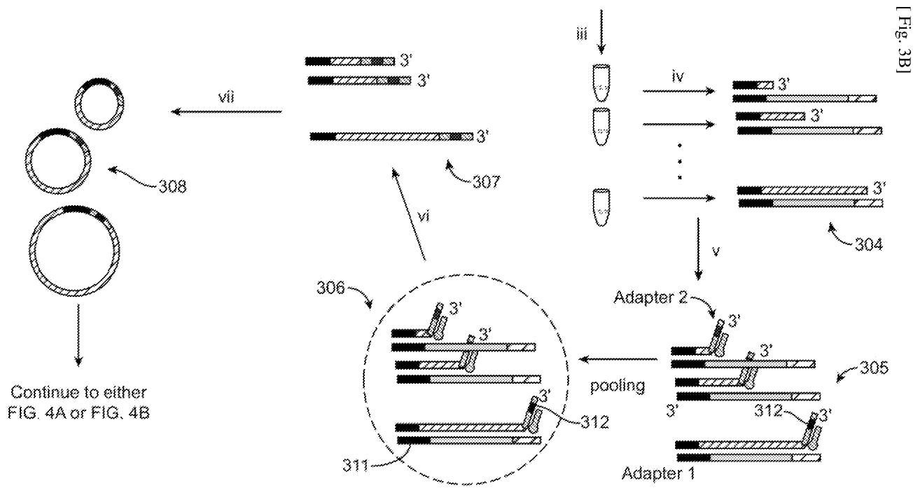

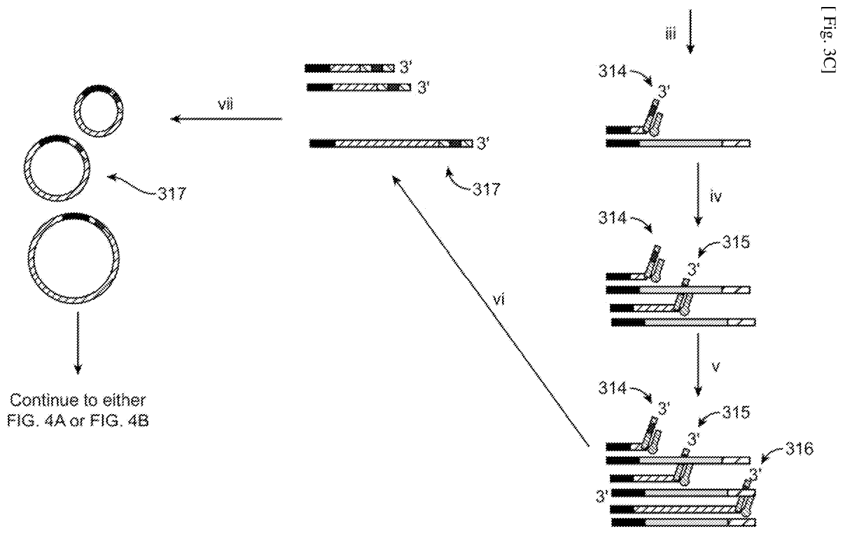

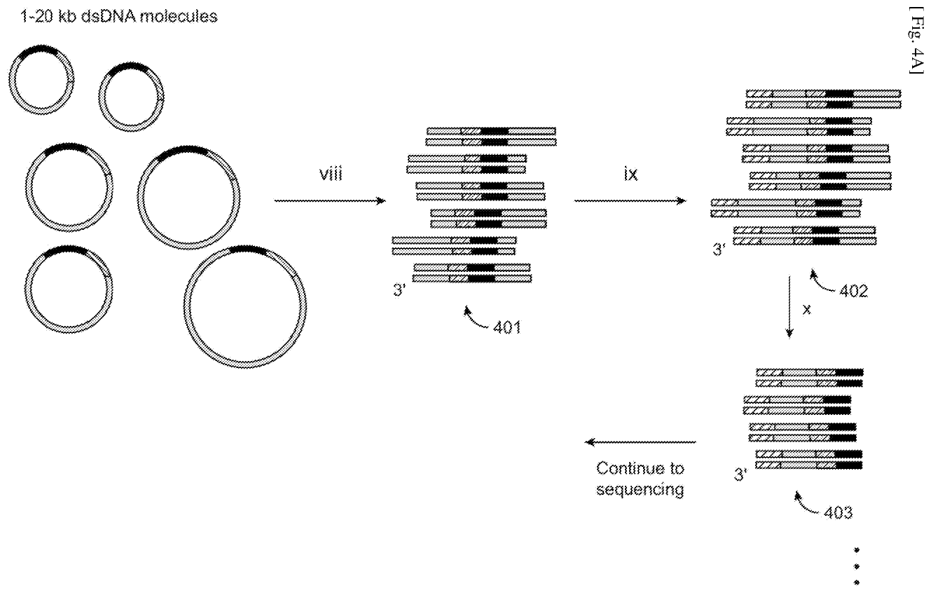

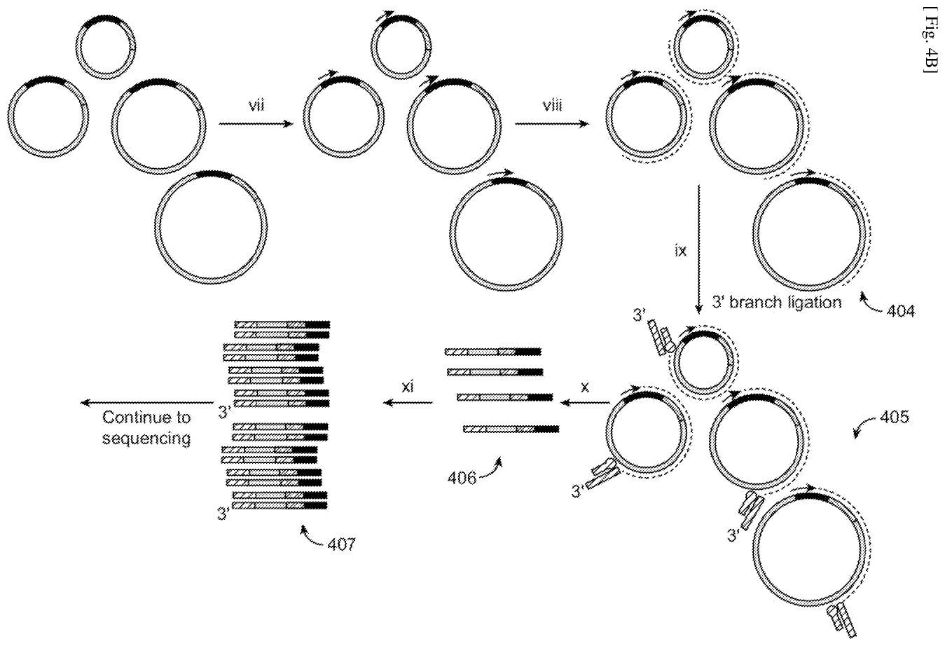

One example of such approaches is illustrated in FIG. 4A. Complementary strands are synthesized using the single-stranded DNA fragments as templates, resulting in formation of a plurality of double-stranded fragments [401]. Step (viii) shows ligating adapters to both ends of the double-stranded fragments to produce adaptered double-stranded constructs [402]. Step (ix) shows performing size selection to generate nucleic acid constructs having lengths that are suitable for sequencing [403].

In some other approaches, linear adaptered double-stranded constructs are generated by extending a primer hybridized to the circle under extension-controlling conditions to produce extended primers of lengths suitable for sequencing. One illustrative example is shown in FIG. 4B, steps (vii) and (viii). Typically, the controlled extension does not result in copying the entire template sequence; rather, the extended primers remain hybridized to the circles [404] with exposed 3-prime ends (i.e., 3-prime recessed ends) that are ready for branch ligation. In some cases, the extended primers have a length within the range of 300-1000 bases, e.g., 300-500 base or 400-600 base to achieve more efficient sequencing. Any short artifact products can be removed through exonuclease treatment or purification. Thus, size selection is not necessary with this approach and all extension products can be used in generating the sequence reads.

A second adapter is then ligated to the recessed 3-prime ends of the extended primers via branch ligation to form adaptered extended primers, each having a second adapter sequence on one end and the primer binding sequence and the barcode sequence on the other end. FIG. 4B, step (ix) [405]. The adaptered extended primers [406] are collected (FIG. 4B, steps (x)) and primer extension is performed using the adaptered extended primers as templates to produce the complementary strands and then form double-stranded DNA fragments [407]. FIG. 4B, steps (xi). The double-stranded DNA fragments can be amplified and sequenced.

A nested set of linear double stranded fragments for each genomic fragment to be sequenced can be generated using the DNA circle-based scheme as described above. Each of the double-stranded DNA fragments in the nested set comprises different target sequence portions of the genomic fragment, and these different target sequence portions together can be assembled to decipher the sequence of the original long DNA molecule. See the section above entitled “assemble sequence information.”

### 6. Linear DNA-Based Scheme

In some approaches, sequence libraries comprising double-stranded adaptered constructs comprising target sequences are generated using a linear DNA-based approach, this is, no DNA circle is generated during the process.

### 6.1 Adding Adapters to Both Ends of Genomic Fragments and Amplify Genomic Fragments

First, adapters are added to both ends of genomic fragments as illustrated in FIG. 2 and also described in section 5.1 above. In some approaches, the genomic fragments are amplified similar to section 5.1 above except that the amplification is carried out by a polymerase in a reaction mixture containing uracils or using primers comprising uracils, thereby producing amplified nucleic acid fragments incorporating uracils in the reaction mixture. One example is shown in FIG. 5, step (i), where the uracils are part of the amplification primers (not shown) and are incorporated into the amplified genomic fragments during amplification.

### 6.2 Creating Nicks

Next, nicks are introduced into amplified genomic fragments. In some approaches, the amplification is in the presence of uracils as described above, and nicks can be introduced to the amplified genomic fragments containing the uracils by contacting them with a uracil-DNA glycosylase. The uracil glycosylase can remove the uracils to form abasic sites. An enzyme (e.g., APE1 or EndoIV) is also added to the reaction to remove the sugar groups from abasic sites. This treatment of the uracil-containing genomic fragments using the enzymes as described above results in nicks the extension products in the region containing uracil bases, each nick flanked by a 5-prime exposed terminus and a 3-prime exposed terminus.

Preferably, uracils are spiked to the amplification reaction after the extension of the amplification primer has passed the barcode region but before reaching an extension length that is approximately the size of the desired read length, also referred to as a length that is suitable for sequencing. The length that is suitable for sequencing may be in a range between 25-1000 bases, depending on the read length dictated by the sequencing methods. In some approaches, this is accomplished by spiking uracils into the reaction mixture after the extension has already been initiated, i.e., when all other components required for amplification have already been added to the reaction mixture. In some approaches, uracils are spiked to the reaction mixture roughly 10 seconds to 10 minutes after the initiation of the extension.

In other approaches, primers used for the implication of the genomic fragment comprise the uracils, which are incorporated into the amplified genomic fragments [501]. In some embodiments, the forward primer comprise one or more uracil. In some embodiments, each forward primer comprises a single uracil such that one nick is generated in each of the double-stranded nucleic acid fragment [502] (after the enzymatic treatment to remove uracils as described above).

### 6.3 Aliquoting

The reaction mixture is then distributed into a plurality of aliquots. See FIG. 5, step (iii)

### 6.4 Nick Translation to Produce a Nested Set of Nucleic Acid Constructs

Next, nick translation is performed with a DNA polymerase with a 5′→3′ exonuclease activity in the aliquots to synthesize DNA strands with newly formed ends (second ends). Nonlimiting examples of DNA polymerases include DNA Poll, Taq, Bst full length, Pfu DNA polymerase. The ends that are opposite to the second ends are the first ends. The extension is controlled such that the DNA strands synthesized in different aliquots have different lengths. Each synthesized DNA strand comprises a first end and a second end, and the DNA strands in different aliquots share the same sequence near the first ends and have different sequences near the second ends [503]. Each of the DNA strands synthesized comprises a target sequence portion with a first end and a second end, the second end being the end formed by the nick translation and the first end being the end opposite from the second end. The DNA strands in different aliquots share the same sequence near the first ends and have different sequence near the second ends. One illustrative example is shown in FIG. 5, step (iv).

### 6.5 Branch Ligation in Individual Aliquots

Adapters (second adapters) are added to the aliquots after the completion of the nick translation reactions. These second adapters are ligated to the second ends of the newly synthesized DNA strands. Each second adapter is partially double stranded and comprises a first adapter oligonucleotide and a second adapter oligonucleotide. The first and second adapter oligonucleotides are complementary and hybridized to each other. During branch ligation, the 5-prime end of the first adapter oligonucleotide is joined to the 3-prime end of a DNA strand synthesized via nick translation as described above (for example, [504] in FIG. 5).

In some approaches, the second adapter comprises a positional barcode that is unique to the aliquot. In some approaches, the aliquots now comprising unique positional barcodes are then combined into one single reaction mixture (for example, [505] in FIG. 5).

In some approaches, the second adapter further comprises an anchoring component for separation of fragments ligated to second adapters from those that are not ligated to the second adapters. In some approaches, the anchoring component allows the adaptered fragments to be captured by solid supports and the captured adaptered fragments can then be isolated from other reagents in solution. In some approaches, the anchoring component can be a biotin, and the solid support is coated with streptavidin. In some approaches, the anchoring component is an oligonucleotide in the second adapter and the solid support is a magnetic bead with oligonucleotides immobilized thereon.

The synthesized DNA strands ligated with the second adapters from different aliquots from (v) are then combined to form in a single mixture (for example, [505]. FIG. 5, step (vi), where the dashed oval represents one single mixture). Subsequent steps are all performed in one single mixture.

### 6.6 Extending the Second Adapter Oligonucleotide of the Second Adapter to Form Double-Stranded Fragments

The branch ligation results in the first adapter oligonucleotide joined to the nucleic acid constructs and the second adapter oligonucleotide not joined but remain hybridized the now joined first adapter oligonucleotide. A primer is then hybridized to the first adapter oligonucleotide and the hybridized primer is extended are to generate double-stranded fragments. In some approaches, the double-stranded fragments so produced have blunt ends. In some approaches, the double-stranded fragments so produced comprises positional barcodes that are unique to individual aliquots. One illustrative example is shown in FIG. 5, step (vii).

### 6.7 Producing Linear Double-Stranded Adaptered Constructs

In some embodiments, the double-stranded DNA molecules having the lengths that are suitable for sequencing are selected. In some approaches, the double-stranded fragments having lengths within a range from 200 bp-1.5 kb, e.g., from 500-1000 bp, are selected. In some approaches. The selected double-stranded fragments are ligated to adapters (“third adapters”) via e.g., blunt-end ligation, thereby producing double-stranded adaptered constructs. See, FIG. 5, Step (viii). The double-stranded adaptered constructs can then be sequenced as disclosed herein.

The sequences near the positional barcode in the double stranded fragments in individual aliquots can be determined by sequencing and sequence reads corresponding to different target sequence portions in individual nucleic acid constructs are assembled to generate sequence information for the entire target sequence.

### 7. Loop-Mediated Complete stLFR

Also provided is a loop-mediated complete stLFR methods to prepare libraries to sequence long DNA molecules. The loop-mediated complete stLFR according to an embodiment of the method comprises preparing a plurality of nested sets of single-stranded nucleic acid constructs using any of the methods disclosed herein. Each single-stranded nucleic acid construct in each nested set comprises a target sequence portion of the long DNA molecule flanked by a first adapter sequence at the 5′ end and a second adapter sequence at the 3′ end (see, e.g., FIG. 13B). The first adapter sequence comprises, from 5′ to 3′, a primer-binding sequence (e.g., 1311 in FIG. 13A), a barcode sequence (e.g., 1319 in FIG. 13A) and a first hybridization sequence (e.g., 1432 in FIG. 13A). The second adapter sequence comprises a second hybridization sequence. The first and the second hybridization sequences are complementary to each other. Each target sequence portion has a first end and a second end, and the distance between the first end and the barcode sequence is shorter than the distance between the second end and the barcode sequence. The single-stranded nucleic acid constructs in each nested set share the same barcode sequence and the single-stranded nucleic acid constructs in different nested sets have different barcode sequences. For each nested set of single-stranded nucleic acid constructs, the target sequence portions in that nested set have identical nucleotide sequences near the first ends and differ from each other by truncations near the second ends, such that each nested set of single-stranded nucleic acid constructs comprises a plurality of target sequence portions having different lengths. Various schemes can be used to generate nested sets of target sequence fragments, as further described below.

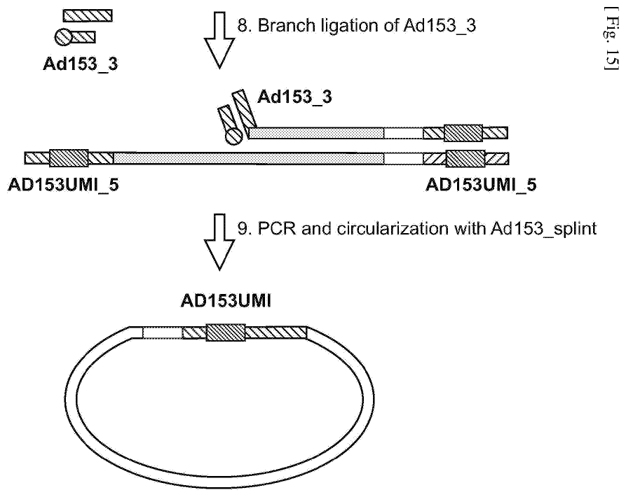

In some embodiments, the method further comprises subjecting the plurality of nested sets of single-stranded nucleic acid constructs to hybridization conditions in a reaction, whereby the first adapter sequence is hybridized to the second adapter sequence, thereby forming a loop (for example, 1431 in FIG. 14). The method further comprises extending the second adapter sequence to copy the barcode sequence and the primer-binding sequence in the first adapter sequence using a DNA polymerase. The method further comprises denaturing the reaction, which results in opening the loop and forming linear single-stranded DNA constructs. Each linear single-stranded DNA construct comprises a barcode sequence and primer-binding sequence at the 3′ end. In some embodiments, the method further comprises annealing a primer to the primer-binding sequence at the 3′ of the linear single-stranded DNA construct and extending the primer to generate an extension product having a length that is suitable for sequencing. Details of the loop-mediated complete stLFR methods are discussed further below.

One exemplary embodiment of the loop-mediated complete stLFR comprises ligating two partially double stranded blunt-end adapters (comprising a first adapter sequence and a third adapter sequence, respectively) to the end-repaired DNA fragments bearing 5′-phosphate groups to prepare adaptered double-stranded genomic fragments each comprising a target sequence flanked by the first adapter sequence and a third adapter sequence. In some embodiments, the third adapter sequence is added to the DNA fragment by nick translation, as described in FIG. 13A and Example 4.

In some embodiments, the double adaptered, double-stranded genomic fragments are amplified. The random nicking and gapping is performed on the double adaptered, double-stranded genomic fragments. Random nicking produced a nested set of fragments having different length of the target sequence portions and sharing the common barcode sequence at the 5-prime. One of such fragments is shown as 1341 in FIG. 13B. [0152]

In the case where an exonuclease is used to open gaps as described above, if necessary, protection of the DNA adapters can be achieved through phosphorothioated bonds between bases and/or modified bases at the 5′ and 3′ ends of the adapters.

A second adapter (e.g., AD153UMI_5R, shown in FIG. 13B) is then ligated to the 3′-side of nicks or ssDNA gaps in the adapter-ligated DNA fragments via branch ligation. This results in a set of fragments having different lengths of the target sequence portions, each flanked by two adapter sequences (for example, AD153UMI_5 and AD153UMI_5R in FIG. 13B). One of such fragments is shown as 1342 in FIG. 13B. The second adapter is a partially double stranded DNA adapter molecule comprising a longer strand (1320.1 in FIG. 13B) and a shorter strand (1320.2 in FIG. 13B). The longer strand has a 5′-phosphate. The longer strand further comprises a first adapter sequence comprising a first hybridization sequence (for example, 1432 in FIG. 14), which is located 3′ relative to the barcode sequence (for example, 1319 in FIG. 14). The longer strand of the second adapter comprises a second adapter sequence comprising a second hybridization sequence (for example, 1433 in FIG. 14). The first and second hybridization sequences are complementary and can hybridize to each other under conditions that are suitable for hybridization, as further disclosed below.

The ligation of the first and/or the second adapter to the DNA fragments via branch ligation can be performed in solution or on beads. In the case where the process is performed on the beads surface, adaptered DNA fragments are preloaded to beads at a high concentration of PEG (5%-15%) before adding other reaction components. In some embodiments, the branch ligation reaction is performed in the presence of additives (e.g., polyethylene glycol or betaine) to increase the activity of ligation and/or the nicking enzyme. This reaction can be incubated at room temperature, 37° C., or cycled between various temperatures, such as 5-15° C. degrees and 37° C. degrees at a pH ranging from 5.0 to 9.0. The incubation may last 5 minutes to several hours. The amount of time and nickase concertation varies depending on the desired number of nicks per DNA fragment. The reaction can be stopped through a DNA purification method (such as Ampure XP beads) if performed in solution, or simply through a washing step with a Tris NaCl buffer containing PEG (5%-15%) if performed on beads.

In some embodiments, after the branch ligation, the DNA fragments are denatured to produce single stranded DNA molecules, each comprising a target sequence portion flanked by the adapter sequences with single stranded hybridization sequences (e.g., the molecule shown in the bottom left of FIG. 13B). In some embodiments, after the branch ligation, the branch-ligated DNA fragments can be heat denatured (90° C.-95° C.). Alternatively, branch-ligated DNA fragments can be denatured by alkaline agents (e.g., 0.05M-0.2M NaOH or KOH) with further neutralization by neutralizing agents (e.g., HCl, Tris-HCl, MOPS). Single-stranded DNA molecules (for example, 1343 in FIG. 13B) comprising target sequence portions flanked by the adapter sequences are formed.

Alternatively, in some embodiments, instead of denaturing, the branch-ligated DNA fragments are digested using one or more dsDNA specific exonucleases possessing 3′-5′ exonuclease activity (e.g., Exonuclease III) to expose 5′ single-stranded first hybridization sequences in the first adapters (e.g., 1432 in FIG. 13 or FIG. 14), available for the hybridization with the second hybridization sequences in the second adapters at the 3′ end of the DNA fragments.

Hybridization between the first and the second hybridization sequences can be carried out in a hybridization buffer containing buffering agents (e.g., Tris-HCl, MOPS, sodium phosphate) and/or salts. In some embodiments, the hybridization buffer also comprises co-factors, such as MgCl2 and dNTPs for subsequent enzymatic reactions.

The DNA hybridization step is followed by extending the hybridized 3′-end of branch adapter (e.g., AD153UMI_5R shown in FIG. 14) to copy the barcode on the first adapter (e.g., AD153 UMI_5 shown in FIG. 14) and the primer binding sequence. In some embodiments, to increase specificity, the extension is performed using one or more DNA polymerases lacking 3′-exonuclease activity. Exemplary DNA polymerases can be used include, but not limited to, Taq DNA polymerase, Klenow Fragment (3′→5′ exo-), and Bst DNA Polymerase, Large Fragment. In some embodiments, the extension is carried out at a temperature that is suitable for the polymerase to carry out the polymerization reaction. In some embodiments, the temperature ranges from 30° C. to 75° C.

The product of linear extension (for example, 1431 in FIG. 14) is in a form of a duplex or partially duplex DNA molecule with a loop. The duplex or partially duplex DNA molecule comprises a double-stranded adapter comprising barcode sequence, and the barcode sequence is attached to a target sequence portion of the long DNA molecule. The duplex or partially duplex DNA molecule is then denatured to open the loop and form a single-stranded fragment (for example, 1441 in FIG. 14) comprising a target sequence portion flanked by the first and the second adapter sequences. By copying the barcodes, the ends of the target sequence portions of different lengths 1411, 1421, 1431, etc. are brought to the proximity of the barcode sequence 1319 on the same DNA strand, such that these ends (having different target sequences) can be sequenced and assembled based on the common barcode sequence 1319. The adapter sequence at the 3-prime of the target sequence portion now comprises a copy of the barcode sequence and a copy of the primer binding sequence. In some embodiments, the primer binding site is recognized by a universal amplification primer. A primer can be annealed to the primer binding sequence and extended to generate an extension product having a length that is suitable for sequencing. The extension product may be ligated to a fourth adapter (for example, Ad153_3 in FIG. 15) via branch ligation, and the fragments (for example, 1510 in FIG. 15) so produced can be amplified by PCR and circularized for DNB sequencing.

Other exemplary schemes for generating nested sets of target sequence fragments are disclosed in sections 8 “Concatemer based methods” and section 9 “combination scheme;” see below. The first scheme starts from ssDNA or dsDNA circles and the second scheme starts from linear ssDNA or dsDNA. Both schemes involve adding adapter sequences to both ends of the molecule of interest, and this can be done through adapter ligation, amplicon PCR, and any number of other strategies. One of these adapters has a barcode sequence that can later be used to identify all reads emanating from this specific DNA fragment/molecule.

### 8. Concatemer Based Methods

### 8.1.1. Concatemer

In some embodiments, provided herein is a concatemer-based method, which produces a nested set of adaptered fragments having target sequence fragments having different length. In some embodiments, the concatemer is produced by rolling circle replication of a single-stranded circular template. The single-stranded circular template can be produced by circularizing a single-stranded DNA molecule using methods well known in the art. For example, circularization can be performed by using a splint oligo having a sequence that is complementary to the adapter sequence at both ends of the molecule and thus brings the 5′ and 3′ ends together for ligation. A DNA concatemer can then be produced by extending a primer annealed to a sequence in the circular template by a DNA polymerase having strand-displacement activity.

The circular DNA template disclosed herein comprises a barcode, a primer sequence, and a target sequence. With circles made, the next step is to form DNA concatemers, e.g., DNA nanoballs (DNBs). The incubation time for making concatemers can range from 20 minutes to several hours. Longer concatemer making times can result in very long concatemers (>100 kb) that may break into separate concatemers. Because of the unique barcode contained within each circle, this breakage is not a problem as all reads coming from these separate concatemers can still be properly identified using the barcode information. Each concatemer comprises a plurality of identical monomers, and each monomer comprises a complement of a target sequence, a complement of the barcode sequence that identifies the DNB, and a primer-binding sequence. The primer-binding sequence comprises a sequence that is complementary to the primer sequence. In some embodiments, the primer-binding sequence is shared by a population of single-stranded concatemers.

### 8.1.2. Producing a Plurality of Extended Primers Separated by Intervals

After concatemers are formed they are converted to dsDNA by extending primers annealed to the primer-binding sequences in the concatemer. In some embodiments, the primers are used in a concentration that is sufficient to ensure that almost all primer-binding sites on the DNB are occupied by extension primers. The extension can then be performed using a polymerase that lacks 5-3′ exonuclease activity and does not possess strand-displacement activity. This results in formation of a DNA complex comprising a plurality of extended primers complementary to the one or more monomers of the DNA concatemer. These extended primers in the DNA complex are hybridized to the DNA concatemer and separated by intervals. See, for example, FIG. 6A. Each extended primer comprises a target sequence fragment. In some embodiments, the primers are DNA primers. In some embodiments, the primers are RNA primers. In some embodiments, the primers are a mixture of RNA primers and DNA primers.

DNA polymerases that lack 5-3′ exonuclease activity and do not possess strand-displacement activity are known, non-limiting examples of which include Klenow exo-, Q5, hemo klen Taq, T7 polymerase, T4 polymerase. Readily available from commercial sources, for example, New England BioLabs, Ipswich, MA.

### 8.1.3. Producing Gaps for Addition of Adapters (in Concatemer Based Methods)

In some embodiments, the intervals between the fragments in the DNA complex as described above are extended (widened) by an exonuclease to form gaps. See, for example, FIG. 6B. This process can be referred to as “gapping” and the exonucleases used in process can be referred to as “gapping enzymes.” Examples of enzymes with 3′ exonuclease activity include DNA Polymerase I, Klenow Fragment (in the absence of nucleotides), Exonuclease III, and others known in the art. Examples of enzymes with 5′ exonuclease activity include Bst DNA polymerase, T7 exonuclease, Exonuclease VIII truncated, Lambda exonuclease, T5 exonuclease, and other exonucleases known in the art. Low processivity exonucleases (i.e., exonucleases that remove nucleotides from the end of a polynucleotide at a relatively low rate) are preferred to open a short gap (e.g. 2-7 bases, 3-10 bases, or 3-20 bases) to allow adapter ligation. Exemplary exonucleases that can be used are shown in Table 1.

TABLE 1
Exemplary exonucleases

| Type of | Direction of | |
|---|---|---|
| DNA substrate | degradation | exonuclease |
| Single- | 5′-->3′ | RecJ Exo VII |
| stranded | | |
| Single- | 3′-->5′ | Exo I, ExoT |
| stranded | | |
| Double- | 5′-->3′ | Bst polymerase, Taq polymerase, T7 |
| stranded | | exonuclease, Exonuclease VIII truncated, |
| | | Lambda exonuclease, T5 exonuclease, |
| Double- | 3′-->5′ | ExoIII, T4 DNA polymerase or Phi29 |
| stranded | | DNA polymerase, DNA Polymerase I, |
| | | Klenow Fragment without nucleotides, |
| | | Exo III |

In scenarios where the fragments are produced by extending RNA primers as described above, RNase H can be added to degrade the RNA primers, thus extending the intervals to form gaps. The gaps will generally have the length of the RNA primer (e.g., 8-40 bases, 10-35 bases, or 10-25 bases). The 5′ terminus and 3′ terminus flanking the interval can be ligated with an L adapter and a 3′ branch ligation adapter, respectively.

FIGS. 6A and 6B exemplify a process of using one or more gapping enzymes (e.g., exonucleases having 3′→5′ exonuclease activity) to widening the intervals (160), resulting in gaps (170). FIG. 7A and FIG. 7B exemplify a process of using one or more gapping enzymes (e.g., exonucleases having 5′→3′ exonuclease activity) to widening the intervals (260), resulting in gaps (270).

To preserve the integrity of the barcode sequence, the exonucleases to be used should only be used to digest the terminus farther away from the barcode sequence. For example, if the barcode sequence is closer to the 5-prime terminus of the target sequence fragment (as in FIG. 6B), then an exonuclease having 3′ to 5′ exonuclease activity is used to digest the fragment starting from 3-prime terminus. On the other hand, if the barcode sequence is closer to the 3-prime terminus of the target sequence fragment (as in FIG. 7B), then an exonuclease having 5′ to 3′ exonuclease activity is used to digest the fragment starting from the 5-prime terminus.

If the primer-binding sequence is located 3-prime to the complement of the barcode sequence in each monomer (i.e., placement of the extension primer is on the 5 prime relative to the barcode sequence in the extended primer), a 3-5′ exonuclease can be added during the ligation step to create target sequence fragments of different sequences by truncation at the 3-prime end, but the identical sequence at the 5-prime end (FIG. 6B). Importantly, this results in every adaptered fragment containing a barcode and coverage across the entire length of the original molecule comprising the target sequence. In addition, the L-oligo adapter can be designed to recognize a portion of the adapter sequence on the concatemer for improved ligation efficiency.

If the primer is located 5-prime to the complement of the barcode sequence in each monomer (i.e., placement of the extension primer is on the 3 prime relative to the barcode sequence in the extended primer), a 5′-3′ exo can be used instead, which generates target sequence fragments having different sequences by truncation at the 5-prime end and the identical sequence at the 3-prime end (FIG. 7B).

If the primer used is a DNA extension primer, a low concentration of exonuclease can be added before adding ligase, adapters, and more exonuclease. This will open most of the intervals into gaps before ligase has a chance to reseal them. If the primer-binding sequence is located 3-prime to the complement of the barcode sequence in each monomer (as in FIG. 6A), an exonuclease having the 3′→5′ exonuclease activity is used (FIG. 6B). If the primer is located 5-prime to the complement of the barcode sequence in each monomer (as in FIG. 7A), an exonuclease having the 5′ →3′ exonuclease activity is used (FIG. 7B).

### 8.1.4. Simultaneous Ligating and Exonuclease Treatment

In some embodiments, an exonuclease is used to generate target sequence fragments having different sizes. Due to the stocastic nature of exonuclease, exonuclease-treatment results in a distribution of different sized, truncated, extended primers, which comprise target sequence fragments. These target sequence fragments have identical nucleotide sequences at the first end and differ from each other by truncations at the second end, such that each set of adaptered fragments comprising the target sequence fragments having different length. In some embodiments, the first ends are the 5-prime termini of the target sequence fragments, as illustrated in (191) in FIG. 6B. In some embodiments, the first ends are the 3-prime termini of the target sequence fragments, as illustrated in (291) in FIG. 7B.

In some embodiments, exonuclease treatment of the extended primers and ligating the extended primers to adapters occur in the same reaction mixture. In some embodiments, ligating comprises ligating at least the branch adapter to the nucleic acid fragment. In some embodiments the ligating includes ligating both the branch adapter and the L-adapter in solution to the extended primers. The following are the exemplary conditions under which exonuclease treatment and ligating can occur in the same reaction.

### Temperature

The reaction may be maintained at a temperature within a range from 5-65° C., e.g., 5-42° C., 10-37° C., or 5-15° C. In some embodiments, the reaction is maintained at room temperature, 37° C. In some embodiments when a thermo-stabile ligase and exonuclease are used, the reaction may be kept at a temperature that is higher than 37° C.

pH

In some embodiments, the pH of the reaction mixture is maintained at a pH within a range from 5.0 to 9.0, e.g., from 7.0 to 9.0, to accommodate all enzymatic functions required for the library preparation. The duration of the exonuclease treatment and ligating reaction may vary depending on the desired size of the nucleic acid fragments and other conditions, e.g., enzyme (including polymerase, exonuclease, or both) concentration, time, temperature, amount of input DNA.

### Time

Typically the duration of the ligating and exonuclease treatment reaction may last from 5 minutes to 5 hours, e.g., 15-90 minutes, or 30-120 minutes. The reaction may be terminated using methods well known in the art. In some embodiments, the exonuclease treatment and ligating are performed in solution, and the reaction can be terminated through a DNA purification method (such as Ampure XP beads, from Beckman Coulter). In some embodiments, the exonuclease treatment and ligation are performed on beads, and the reaction can be terminated by washing the beads with a buffer (e.g., a Tris NaCl buffer) to remove the enzymes and components required for the nicking and ligating reactions.

### 8.1.5. Ligation of Adapters

As discussed above and illustrated in FIG. 6B, extending primers (130) annealed to the adapter sequence in the DNA concatemer generate a plurality of extended primers (150) each having a 5′ terminus and a 3′ terminus. In some embodiments, each of the at least some of the extended primers is ligated with two adapters, one at either terminus. In some embodiments, an L-adapter is ligated to the 5′ terminus and a branch adapter is ligated to the 3′ terminus of the extended primer. The result is a plurality of adaptered fragments having two different adapter sequences; and all of the adaptered fragments produced in a reaction have the same defined arrangement (e.g., an L-adapter at 5′ and a branch adapter at 3′).

### 9. Combinational Scheme

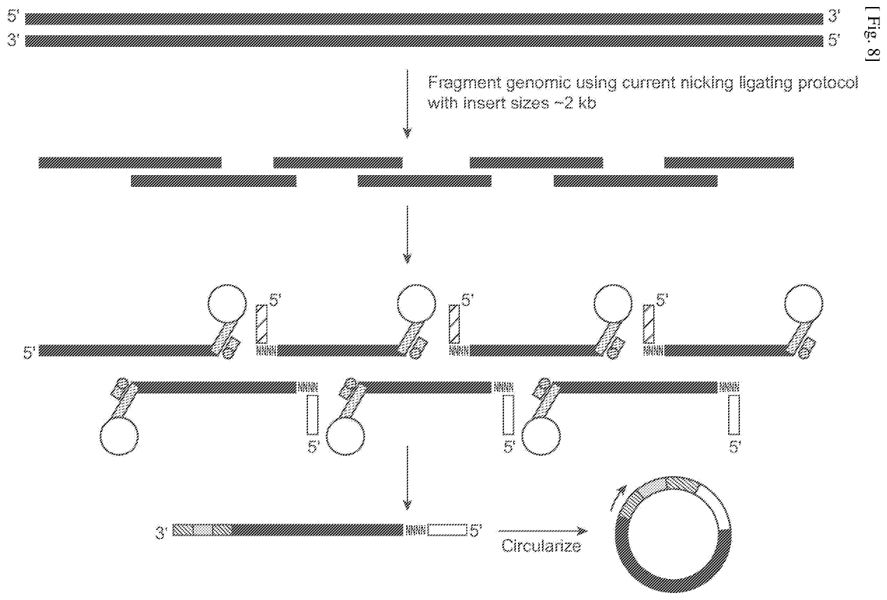

In some embodiments, the method disclosed herein can be combined with the stLFR method to sequence long genomic DNAs, for example, a genomic fragment having a length of 20 kb to 200 kb. In this case, we would perform stLFR as disclosed in, e.g., U.S. Pat. No. 9,328,382B2, PCT publication WO2023001262, followed by the methods described above and adjust the size of barcoded fragments from about 300 to 1000 bp or 500 bp to 1500 bp or 1 kb to 3 kb. An advantage of using longer inserts is tolerance on bias in enzymes enabling stLFR cobarcoding (e.g., transposase or DNA nicking enzymes). It is also easier to remove stLFR adapter-adapter artefacts by size selection (i.e., removing DNA less than 300 bp in length). The barcode provided by the bead would become the barcode used for each circle. After performing the processes above, each 2 kb fragment would have greater than 1× read coverage and could them be combined with other fragments sharing the same stLFR barcode to create long fragments (up to several megabases is possible) with close to 1× or more coverage across the entire fragment (5-15× average read coverage). An exemplary method according to this embodiment is shown in FIG. 8.

Overall, 10× or more coverage in long fragments per each haplotype with each long fragment receiving ˜5-10× read coverage would require 50-100× total read coverage per haplotype. Such complete and accurate WGS will become more affordable with further cost reduction of MPS (NGS).

In some embodiments, the method starts with any linear DNA molecule with an adapter on at least one end. In some embodiments, the method starts with PCR amplicons, which can provide enough copies of each barcoded molecule. In some embodiments, this process can be performed in solution. In some embodiments, this method can be performed on beads, on which the one terminus (5-prime or 3-prime) of the adapter of the linear DNA molecule is immobilized thereon (FIG. 9) and the adapter comprises a unique barcode. In some embodiments, the barcoded fragment comprises a barcode sequence, a target sequence, and a primer binding sequence, wherein 3-prime terminus of the barcoded fragment is immobilized on a bead. In some embodiments, the barcoded fragment comprises a barcode sequence, a target sequence, and a primer binding sequence, wherein 5-prime terminus of the barcoded fragment is immobilized on a bead. In some embodiments, the primer-binding sequence is 3 prime relative to the barcode sequence.

Polynucleotides (e.g., barcoded fragments) can be immobilized on the beads in a variety of ways, including covalent and non-covalent attachment. In some embodiments, the 3′ or 5′ end of the adapter of the polynucleotide is attached to a biotin and the barcoded fragments are captured onto streptavidin-coated beads. In some embodiments, the polynucleotide is joined to a substrate (e.g., a bead), that is, one terminus of the polynucleotide directly contacts or is linked to the substrate. For example, a surface may have reactive functionalities that react with complementary functionalities on the polynucleotide molecules to form a covalent linkage. Long DNA molecules, e.g., several nucleotides or larger, may also be efficiently attached to hydrophobic surfaces, such as a clean glass surface that has a low concentration of various reactive functionalities, such as —OH groups. In still another embodiment, polynucleotide molecules can be adsorbed to a surface through non-specific interactions with the surface, or through non-covalent interactions such as hydrogen bonding, van der Waals forces, and the like.

In some embodiments, a polynucleotide (e.g., a barcoded fragment) is immobilized to a surface through hybridizing to a capture oligonucleotide on the surface and forming complexes, e.g., double-stranded duplexes or partially double-stranded duplexes, with component of the capture oligonucleotide.

In some embodiments, the method uses a primer comprising a primer sequence that is complementary to the primer sequence in the barcode fragment. In some embodiments, the primer is a tailed primer, which comprises a tail that is not complementary to the barcoded fragment. In some embodiments, the tail comprises a common adapter sequence. FIG. 9.

In some embodiments, the extension is controlled such that the polymerase extends the primer past the barcode region on the barcoded fragment. In some embodiments, the polymerase extends the primer past the barcode region by a length roughly equal to length that is suitable for sequencing (aka., a sequencing read length), for example in the range of 25-1000 bases. FIG. 9. The extension product, i.e., the extended primer can be separated from the barcoded fragment and leaving the barcoded fragment as a template for subsequent cycle of extension reactions. Various ways of controlling extension reaction can be used, which is further described below in section 3.4 (“Controlled extension”). In some embodiments, the primer extension (of one or more cycles) can be controlled in a manner so that the subsequent cycle of extension produces longer or shorter extension product than that of the previous cycle of extension. In some embodiments, the controlled extension is performed in the presence of reversible terminators. In some embodiments, the controlled extension is performed by using a different polymerase that is capable of performing a longer or shorter extension. One of the advantages of using terminators is that the length of additional polymerization can be controlled by the concentration of the terminators which can be easier to control than time. At the end of the polymerization, the terminators can be reversed, the beads washed, In some embodiments, the beads can be washed in a buffered NaCl solution. This is then followed by 3′ branch ligation of a branch adapter (e.g., 440 in FIG. 9). After denaturation of the DNA using either heat or alkaline conditions, the supernatant can be collected, purified.

This process, including, e.g., the primer extension and denaturation, can be performed many rounds to generate a nested set of adaptered fragments comprising target sequence fragments of varying length in such a way that the entire original DNA molecule is covered. These adaptered fragments also share the same barcode sequence. This method will result in variable target sequence fragments having sizes ranging from about 100 bp to 5000 bp, from 100 bp to 3000 bp, from 100 bp to 1000 bp, from 100 bp to 750 bp, or from 100 bp to 500 bp.

The target sequence fragments generated above can be circularized for DNA nanoballs (DNB) preparation and sequencing. As described above, these target sequence fragments have identical nucleotide sequences at the first end (the end that is closer to the barcode sequence) and differ from each other by truncations at the second end. In some embodiments, the sequencing is a paired-end sequencing comprising sequencing from either terminus of the same DNA fragment. In some embodiments, first read reads are produced by extending a sequencing primer annealed to the adapter sequence that is closer to the first end of the target sequence fragment than the second end (“first read sequencing”), and second sequencing reads are produced by extending a sequencing primer annealed the adapter sequence that is closer the second end of the target sequence fragment than the first end (“second read sequencing”). The first read sequencing will produce the barcode sequence. The second read sequencing will produce overlapping reads to substantially or completely cover molecules up to 500 bp or 700 bp or 1000 bp in length. These overlapping sequencing reads would be clustered based on the barcode sequence determined by the first read sequencing in a de novo assembly.

In some embodiments, in order to more efficiently cover the entire molecule but avoid producing fragments that have excessive lengths (e.g., over 700 bp, or over 1000 bp, or over 1500 bp or over 2000 bp or over 3,000 bp) uracils are incorporated in the middle portion of the extension step. For example, uracils may be added to the reaction after the extension has passed the barcode region but before reaching an extension length that is approximately the size of the desired read length (e.g., 25-1000 bases depending on the read length dictated by the sequencing methods). This can be achieved by extending the primer for a first extension period, then spiking uracil into the reaction mixture to allow the primer to continue extending for second extension period, then washing the beads, and new uracil-free deoxynucleotide mix (normal deoxynucleotides) are added to the reaction to allow the primer to extend further for a third extension period (FIG. 10A-10B). In some embodiments, this third extension reaction (uracil-free extension) is performed in the presence of reversible terminators, e.g., using a mixture of normal nucleotides and reversible terminators.

After the extension reactions are completed, the terminators, if used, can be reversed, the beads washed, and the extension product is ligated with a 3′ branch ligation adapter. Next, a uracil glycosylase can be added to remove the uracils to form abasic sites and an enzyme that can remove the sugar groups from abasic sites are added to the reaction. This will result in the fragmenting of the extension products in the region containing uracil bases. Non-limiting examples of enzymes that are capable of removing sugar groups from abasic sites include APE1 or EndoIV. Removing these fragmented products will leave gaps that is flanked by a 5-prime exposed terminus and a 3-prime exposed terminus. L-adapter and an internal branch adapter are ligated to the exposed 5-prime terminus and the exposed 3-prime terminus. In some embodiments, the sequences of the L-adapter, the internal branch adapter, the adapter sequences at the 5′ and 3′ ends of the extended fragment are all distinguishable from one another. The 5′ and 3′ ends of these two adapters can then be joined via a splint oligonucleotide and ligated by T4 ligase to rejoin the 5′ and 3′ sides of the extended fragment (the single-stranded part of the template molecule will fold bringing two adapters in close proximity to hybridize the splint oligonucleotide, FG. 5B).

Now this product can be denatured, separated from the beads, and collected. In some cases, the beads can be reused for one or more cycles. See FIG. 10C. The denatured products from all rounds are collected and sequenced. This procedure decreases the overall length of the target sequence fragments in each of the adaptered fragment by removing a section of the middle of the extension product. See FIG. 10C. FIG. 11 shows the shorted target sequence fragments having sequences that correspond to different regions of the target sequence of the original molecule. This allows read coverage by MPS for molecules up to 1500 bp, 2000 bp, 3000 bp, 4000 bp or 5000 bp. For 3 kb molecules to get 10× coverage with 300 base per read, 100 such reads are needed, because of the losses in library preparation and DNB or cluster making it preferable that the library process would start with at least 300, or at least 1000, or at least 10,000 copies of each DNA molecule. The copy number can be reduced by 2-10 fold or more, if beads are reused. If longer sequencing reads are available (e.g., 600 base) and 25-100 reuses of shorter template molecules (e.g., 1500 bp) are performed, it is possible to this process can be run without initially amplifying molecules by linear or exponential PCR or other methods. However, amplified molecules allow multiple reactions (2 or more, 3 or more, 4 or more, 2-6, or 4-8) in parallel with longer and longer regions of uracils to best cover all regions of DNA molecules having the length in the range from 1 kb to 10 kb, for example, from 1 kb to 5 kb, or from 1 kb to 3 kb.

Another exemplary solution to the problem (i.e., the original molecule comprising the target sequence being too long for sequencing) is to use a first branch adapter having a degenerate sequence region at the 3-prime portion. This first branch adapter is ligated the extended tailed primer formed after a first extension as described above. The first extension is controlled such that the primer is extended past the barcode region. In some embodiments, the degenerate sequence region comprises 3-10, for example 3-8, 5-10, or 6-10 degenerate nucleotides. The first branch adapter can hybridize to random locations in the barcoded fragment through the degenerate sequence region, which result in skipping of replication of some random portion of the barcoded fragment. A second controlled extension is then performed by extending the 3-prime terminus of the first branch adapter. The second extension may be performed such that 100-300 bases are added to said 3-prime terminus to form a second extension product. A second branch adapter can then be ligated to the 3-prime terminus of the second extension product to produce an adaptered fragment. See FIG. 12.

In some embodiments, the adaptered fragments are denatured and released from the bead. The barcoded fragments can be used as extension template for the additional cycles of extensions to generate more adaptered fragments. The first and second extensions in each cycle are controlled so that the adaptered fragments produced from the cycles having overlapping target sequence fragments. These adaptered fragments can be sequenced and sequencing reads of the overlapping target sequence fragments can be assembled to generate the sequence information for the entire target sequence.

In some embodiments, the barcoded fragments have been amplified such that multiple copies of the barcoded fragment are used as templates for extension (e.g., for extending a primer annealed to the barcode fragment). In some embodiments, these multiple copies are immobilized on the same bead. In some embodiments, these multiple copies are immobilized on more than one bead. These copies can be identified by the same barcode they share. In this embodiment, one cycle (including the first extension, ligation with the first branch adapter, the second extension, and ligation with the second branch adapter) is often sufficient to generate overlapping target sequence fragments. But if needed, the extension products can be denatured and released from the beads, and the barcoded fragment can be reused for the additional cycles of generating additional adaptered fragments as described above.

### 10. Exemplary Embodiments of the Disclosure

Embodiment 1 is a method of producing single-stranded adaptered constructs for sequencing comprising: preparing a plurality of nested sets of single-stranded nucleic acid constructs, wherein each single-stranded nucleic acid construct in each nested set comprises a target sequence portion flanked by a first adapter sequence at the 5′ end and a second adapter sequence at the 3′ end, wherein the first adapter sequence comprises, from 5′ to 3′, a primer-binding sequence, a barcode sequence and a first hybridization sequence and the second adapter sequence comprises a second hybridization sequence, wherein the first and the second hybridization sequences are complementary to each other, wherein each target sequence portion has a first end and a second end, wherein the distance between the first end and the barcode sequence is shorter than the distance between the second end and the barcode sequence, wherein the single-stranded nucleic acid constructs in each nested set share the same barcode sequence and the single-stranded nucleic acid constructs in different nested sets have different barcode sequences, and wherein for each nested set of single-stranded nucleic acid constructs, the target sequence portions in that nested set have identical nucleotide sequences near the first ends and differ from each other by truncations near the second ends, such that each nested set of single-stranded nucleic acid constructs comprises a plurality of target sequence portions having different lengths.

Embodiment 2 is the method of Embodiment (s) 1, wherein the method further comprises subjecting the plurality of nested sets of single-stranded nucleic acid constructs to hybridization conditions, whereby the first adapter sequence is hybridized to the second adapter sequence, thereby forming a loop.

Embodiment 3 is the method of Embodiment (s) 2, wherein the method further comprises extending the second adapter sequence to copy the barcode sequence and the primer-binding sequence in the first adapter sequence using a DNA polymerase to form an extension product.

Embodiment 4 is the method of Embodiment (s) 3, wherein the method further comprises denaturing the extension product to open the loop, thereby forming linear single-stranded DNA constructs, wherein each linear single-stranded DNA construct comprises a barcode sequence and primer-binding sequence, wherein the primer-binding sequence is located 3′relative to the barcode sequence. In some embodiments, the primer-binding sequence at the 3′end of the linear single-stranded DNA construct.

Embodiment 5 is the method of Embodiment (s) 4, wherein the method further comprises annealing a primer to the primer-binding sequence at the 3′ of the linear single-stranded DNA construct and extending the primer to generate an extension product having a length that is suitable for sequencing.

Embodiment 6 is a method of producing single-stranded DNA circles comprising single-stranded adaptered constructs for sequencing comprising: preparing a plurality of nested sets of single-stranded nucleic acid constructs, wherein each single-stranded nucleic acid construct in each nested set comprises a target sequence portion flanked by a first adapter sequence and a second adapter sequence, wherein the first adapter sequence comprises a barcode sequence and a primer-binding sequence, wherein each target sequence portion has a first end and a second end, wherein the distance between the first end and the barcode sequence is shorter than the distance between the second end and the barcode sequence, wherein the single-stranded nucleic acid constructs in each nested set share the same barcode sequence and the single-stranded nucleic acid constructs in different nested sets have different barcode sequences, wherein for each nested set of single-stranded nucleic acid constructs, (a) the target sequence portions in that nested set have identical nucleotide sequences near the first ends and differ from each other by truncations near the second ends, such that each nested set of single-stranded nucleic acid constructs comprises a plurality of target sequence portions having different lengths, and (b) circularizing the single-stranded nucleic acid constructs in each nested set to produce the single-stranded DNA circles, in which the first adapter sequence and the second adapter sequence are joined.

Embodiment 7 is the method of any one of Embodiments 1-6, wherein each nested set of single-stranded nucleic acid constructs is prepared by: (i) preparing adaptered double-stranded genomic fragments each comprising a target sequence flanked by the first adapter sequence and a third adapter sequence, (ii) amplifying the adaptered double-stranded genomic fragments to produce amplified genomic fragments by using primers hybridized to the first and third adapter sequences, (iii) contacting the amplified genomic fragments from (ii) with a nicking agent to produce nicks in the target sequences in one strand of the amplified genomic fragments, (iv) ligating a second adapter comprising the second adapter sequence at the nicks in (iii) via branch ligation to form ligated products, and (v) denaturing the ligated products from (iv) to form the single-stranded nucleic acid constructs, each comprising the first adapter sequence and the second adapter sequence.

Embodiment 8 is the method of any one of Embodiments 1-6, wherein each nested set of single-stranded nucleic acid constructs is prepared by (i) preparing adaptered double-stranded genomic fragments each comprising a target sequence flanked by the first adapter sequence and a third adapter sequence, (ii) amplifying the adaptered double-stranded genomic fragments to produce amplified genomic fragments, (iii) distributing the amplified genomic fragments into a plurality of aliquots, (iv) denaturing the amplified genomic fragments in (iii) to prepare single-stranded genomic fragment, wherein at least some of the single-stranded genomic each comprising the primer-binding sequence, (iv) extending a primer hybridized to the primer-binding sequence under extension-controlling conditions such that the lengths of extension products from different aliquots are different, thereby producing extension products having newly formed ends, and the extension products have different sequences near the newly formed ends in different aliquots, wherein each extension product comprises a target sequence portion, and (v) ligating a second adapter comprising the second adapter sequence at the newly formed ends via branch ligation in each aliquot, thereby producing the single-stranded nucleic acid constructs, each comprising the first adapter sequence and the second adapter sequence.

Embodiment 9 is the method of any one of Embodiments 1-6, wherein each nested set of single-stranded nucleic acid constructs is prepared by (i) preparing adaptered double-stranded genomic fragments each comprising a target sequence flanked by the first adapter sequence and a third adapter sequence, (ii) amplifying the adaptered double-stranded genomic fragments to produce amplified genomic fragments, (iii) distributing the amplified genomic fragments into a plurality of aliquots, (iv) adding a double-stranded DNA nuclease with 3′ 5′ nuclease activity the plurality of aliquots under controlled conditions such that the lengths of products remaining after the double-stranded DNA nuclease digestion in different aliquots are different, thereby producing digestion products having newly formed ends with different sequences in different aliquots, wherein each digestion product comprises a target sequence portion, and (v) ligating a second adapter comprising the second adapter sequence at the newly formed ends via branch ligation in each aliquot, thereby producing the single-stranded nucleic acid constructs, each comprising the first adapter sequence and the second adapter sequence.

The method of any one of Embodiments 1-6, wherein each nested set of single-stranded nucleic acid constructs is prepared by (i) preparing adaptered double-stranded genomic fragments each comprising a target sequence flanked by the first adapter sequence and a third adapter sequence, (ii) amplifying the adaptered double-stranded genomic fragments to produce amplified genomic fragments, (iii) denaturing the amplified genomic fragments to prepare single-stranded genomic fragments, wherein at least some of the single-stranded genomic fragments each comprising the primer-binding sequence, (iv) for each single-stranded genomic fragment, extending a primer hybridized to the primer-binding sequence for a first period of time to produce an extended primer, wherein the extension is incomplete such that the length of the extended primer is a fraction of the length of the single-stranded genomic fragment, wherein the extended primer comprises a target sequence portion, and ligating a second adapter via branch ligation to the end of the extended primer formed by the extension, thereby producing single-stranded nucleic acid constructs in one reaction mixture, each comprising the first adapter sequence and the second adapter sequence, (v) repeat step (iv) for multiple rounds, for each round, the primer is further extended for an additional period of time, and an additional adapter having a unique positional barcode is ligated to the further extended primer, wherein the additional adapter is used in a molar amount that is a fraction of the total molar amount of the amplified genomic fragments, thereby producing a mixture of nested set of single-stranded nucleic acid constructs.

Embodiment 11 is the method of Embodiment 8, wherein the target sequence comprises repetitive sequences, wherein the second adapter comprises a positional barcode sequence that is unique to each aliquot, wherein the single-stranded nucleic acid constructs formed in (v) in different aliquots comprise different positional barcode sequence, and the single-stranded nucleic acid constructs in the same aliquot share the same positional barcode sequence.

Embodiment 12 is the method of Embodiment 6, wherein the primer-binding sequence is 3-prime in relation to the barcode sequence.

Embodiment 13 is the method of Embodiment 6, wherein the method further comprises (vi) fragmenting the single-stranded DNA circles to produce a plurality of single-stranded DNA fragments, wherein at least some of which comprise the barcode sequence, (vii) producing double-stranded DNA fragments from the single-stranded DNA fragments from step (vi), (vii) ligating a second adapter to each of the double-stranded DNA fragments from step (vii), thereby producing double-stranded adaptered fragments.

Embodiment 14 is the method of Embodiment 13, the method further comprises (viii) amplifying the double-stranded adaptered fragments, and optionally (ix) selecting the amplified double-stranded adaptered fragments having lengths within a range of 300-1000 bases.

Embodiment 15 is the method of Embodiment 6, wherein the method further comprises (vi) hybridizing a primer to the primer-binding sequence in each of the single-stranded DNA circles, (vii) extending the primer under extension-controlling conditions using each of the single-stranded DNA circles as templates, wherein the extending produces an extended primer hybridized to single-stranded DNA circles, thereby producing a plurality of extended primers having different lengths, wherein said each of the extended primers comprises the barcode sequence and the primer-binding sequence, (viii) ligating a second adapter to the plurality of extended primers via branch ligation to produce adaptered extended primers.

Embodiment 16 is the method of any one of Embodiments 6-15, wherein the method further comprises amplifying the adaptered extended primers to produce amplified double-stranded fragments, selecting the amplified double-stranded fragments having lengths within a range from 300 bases to 1000 bases, and sequencing the selected amplified double-stranded adaptered fragments.

Embodiment 17 is the method of Embodiments 1-16, wherein the single-stranded DNA circles are prepared in solution, without solid supports.

Embodiment 18 is the method of Embodiment 6, wherein the first end or the second end is attached to a solid support.

Embodiment 19 is a method of producing double-stranded adaptered constructs for sequencing, wherein the method comprises: (i) amplifying a plurality of genomic fragments, each genomic fragment comprising a target sequence, to produce a plurality sets of amplified nucleic acid fragments in a mixture, wherein the amplified nucleic acid fragments in each set share the same target sequence, optionally the amplification is performed using target-specific primers, for each set, the method further comprises (ii) contacting the amplified nucleic acid fragments with an enzyme, wherein the enzyme introduces breaks in the amplified nucleic acid fragments, (iii) distributing the mixture of fragments into a plurality of aliquots, (iv) performing nick translation on the aliquots of fragments to synthesize DNA strands under conditions such that the DNA strands synthesized in different aliquots have different lengths, wherein each of the DNA strands comprises a target sequence portion with a first end and a second end, and wherein the DNA strands in different aliquots share the same sequence near the first ends and have different sequence near the second ends, (v) for each aliquot, ligating second adapters to the second ends of the DNA strands synthesized in (iv) via branch ligation, wherein each second adapter is a partially double stranded adapter comprising a first adapter oligonucleotide and a second adapter oligonucleotide, wherein both the first adapter oligonucleotide and a second adapter oligonucleotide are complementary and hybridized to each other, wherein each of the second adapters comprises a positional barcode sequence, wherein each ligation comprises joining a 5-prime end of the first adapter oligonucleotide of the second adapter to a second end of the synthesized DNA strand, wherein the first adapter oligonucleotides ligated to the second ends of the synthesized DNA strands in different aliquots comprise different positional barcode sequence, and the first adapter oligonucleotides ligated to the second ends of the synthesized DNA strands in the same aliquot share the same positional barcode sequence, (vi) combining the synthesized DNA strands ligated with the second adapters from different aliquots from (v) in a single mixture, (vii) extending a primer hybridized to the first adapter oligonucleotides that have been ligated to the synthesized DNA strands to produce double-stranded fragments having blunt ends, and (viii) optionally selecting the double-stranded fragments of (vii) with a size within a range from 200 bp-1.5 kb (will disclose a range around the optimal 500-1000 bp in the specification) from the single mixture, and (ix) ligating a third adapter to the blunt ends of the double-stranded fragments, thereby producing double-stranded adaptered constructs.

Embodiment 20 is the method of Embodiment 19, wherein step (i) comprises amplifying the plurality of genomic fragments in a mixture comprising uracils, thereby producing amplified nucleic acid fragments with uracils incorporated, and wherein step (ii) comprises contacting the amplified nucleic acid fragments with a uracil-DNA glycosylase, wherein the uracil-DNA glycosylase removes the uracils from the amplified genomic fragments.

Embodiment 21 is the method of Embodiment 19, wherein the amplifying the plurality of genomic fragments in step (i) is performed using primers comprising the uracils, thereby producing the plurality sets of amplified nucleic acid fragments comprising uracil.

Embodiment 22 is the method of Embodiment 21, wherein each of the plurality of genomic fragments is amplified using a forward primer and a reverse primer, and wherein each forward primer comprise one or more uracils.

Embodiment 23 is the method of Embodiment 22, wherein each of the plurality of genomic fragments is amplified using a forward primer and a reverse primer, and wherein each reverse primer comprise a single uracil.

Embodiment 24 is the method of Embodiment 19, wherein step (ii) comprises contacting the amplified genomic fragments with an endonuclease, wherein the endonuclease cuts the amplified genomic fragments at random.

Embodiment 25 is the method of Embodiment 24, wherein the endonuclease is EndoIV or APE1.

Embodiment 26 is a reaction mixture comprising the single-stranded DNA circles produced in claim 6.

Embodiment 27 is a reaction mixture comprising the combined synthesized DNA strands from step (vi) of the claim 18.

Embodiment 28 is a method for preparing a plurality of nested sets of adaptered fragments, wherein each adaptered fragment is a single-stranded nucleic acid comprising a target sequence fragment having a first end and a second end, a 5-prime adapter sequence, a 3-prime adapter sequence, a primer sequence, and a barcode sequence, wherein in each nested set of adaptered fragments, the target sequence fragments have identical nucleotide sequences at the first end and differ from each other by truncations at the second end, such that each nested set of adaptered fragments comprises a plurality of target sequence fragments having different length, wherein the first end is closer to the barcode sequence than the second end, wherein the method comprises: (a) providing, in a reaction, a population of single-stranded DNA concatemers, wherein each concatemer comprises a plurality of identical monomers, and each monomer comprises a complement of a target sequence, a complement of the barcode sequence that identifies the concatemer, and a primer-binding sequence shared by the population of single-stranded concatemers, wherein the primer-binding sequence comprises a sequence that is complementary to the primer sequence, wherein both the primer-binding sequence and complement of the barcode sequence are 3-prime to the complement of the target sequence; (b) annealing primers comprising the primer sequence to primer-binding sequences of multiple monomers of each of plurality of the concatemers; (c) extending at least some of the primers hybridized to the primer-binding sequences with a DNA polymerase that has 5′-->3′ exonuclease activity and does not have strand displacement activity, wherein the extending produces a plurality of extended primers, each said extended primer comprising a target sequence fragment with barcode sequences and primer sequences, wherein the extended primers are hybridized to the concatemer; wherein the extended primers are separated by intervals, and (d) contacting the plurality of the extended primers with a 5-prime adapter comprising the 5-prime adapter sequence, a 3-prime adapter comprising the 3-prime adapter sequence, a DNA ligase, and an exonuclease having single-strand DNA exonuclease activity under conditions in which the exonuclease degrades a portion of the target sequence fragments in the extended primers, to produce shortened extended primers, the 5-prime adapters are ligated to the 5′ end of the shortened extended primers, and the 3-prime adapters are ligated to the 3′ end of the shortened extended primers, thereby producing a group of plurality of nested sets of adaptered fragments.

Embodiment 29 is the method of Embodiment 28, wherein the population of single-stranded DNA concatemers are produced by rolling circle replication of circular templates, wherein each of the circular templates comprises the target sequence, the barcode sequence and the primer sequence.

Embodiment 30 is the method of Embodiment 28, wherein the 5-prime adapter is an L-adapter and the 3-prime adapter is a branch adapter.

Embodiment 31 is the method of Embodiment 28 wherein the method further comprises adding a nuclease to extend the intervals formed in step (c), wherein the nuclease has single-strand exonuclease activity.

Embodiment 32 is the method of Embodiment 31, wherein the at least some of the primers are RNA primers, and wherein the nuclease is an RNAse H, wherein the RNAse H digests the RNA primers, thereby extending the intervals.

Embodiment 33 is the method of Embodiment 28, wherein the primer-binding sequence is located 3-prime to the complement of the barcode sequence in step (a), wherein the exonuclease has a 3′ 5′ exonuclease activity, and wherein the barcode sequence in each of the set of adaptered fragments is located 5-prime relative to the target sequence fragment.

Embodiment 34 is the method of Embodiment 28, wherein the primer-binding sequence is located 5-prime relative to the complement of the barcode sequence in step (a), wherein the exonuclease has a 5′ 3′ exonuclease activity, and wherein the barcode sequence is 3-prime relative to the target sequence fragment in each of the adaptered fragments.

Embodiment 35 is the method of any one of the preceding claims, wherein the both the 5-prime adapter and the 3-prime adapter are in solution.

Embodiment 36 is the method of Embodiment 35, wherein the reaction is free of solid supports.

Embodiment 37 is the method of any one of the preceding claims, wherein the target sequence has a length between 500 bases to 50 kilobases.

Embodiment 38 is the method of Embodiment 30, wherein the branch adapter comprises a double-stranded blunt end comprising a 5′ terminus of one strand and a 3′ terminus of the complementary strand and wherein the 5′ terminus of the strand in the double-stranded blunt end is ligated to the 3′ terminus of at least one of the extended primers via branch ligation.

Embodiment 39 is the method of Embodiment 30, wherein the L-adapter comprises 1-10 degenerated bases at 3-prime.

Embodiment 40 is a method for preparing a plurality of nested sets of adaptered fragments, wherein each adaptered fragment is a single-stranded nucleic acid comprising a target sequence fragment having a first end and a second end, a 5-prime adapter sequence, a 3-prime adapter sequence, a primer-binding sequence, and a complement of a barcode sequence, wherein in each nested set of adaptered fragments, the target sequence fragments have identical nucleotide sequences at a first end and differ from each other at a second end, such that each nested set of adaptered fragments comprises a plurality of target sequence fragments having different length, wherein the first end is closer to the barcode sequence than the second end, wherein the method comprises (a) providing a barcoded fragment comprising a barcode sequence, a target sequence, and a primer binding sequence, wherein the barcoded fragment is immobilized on a bead at one terminus, (b) annealing a primer comprising the 5-prime adapter sequence to the primer-binding sequence in the barcoded fragment, wherein the 5-prime adapter sequence comprises i) a complement of the barcode sequence, and ii) a primer sequence complementary to the primer binding sequence in the barcoded fragment, (c) extending the primer to produce an extended primer comprising a target sequence fragment and a complement of the barcode sequence, (d) contacting the extended primer with a branch adapter comprising the 3-prime adapter sequence to produce an adaptered fragment, (e) separating the adaptered fragment from the barcoded fragment that remains immobilized on the bead, and (f) repeating steps (b)-(e) for one or more cycles under extension-controlling conditions to produce one or more adaptered fragments, wherein the adaptered fragment generated from step (e) and the adaptered fragments generated from step (f) and constitute the nested set of adaptered fragments, and wherein the adaptered fragments in each nested set comprise target sequence fragments having different length.

Embodiment 41 is the method of Embodiment 40, wherein the primer is extended under extension-controlling conditions with uracils in one or more cycle of extensions s to produce the extended primer, thereby producing the adaptered fragment incorporating the uracils at 5 prime portion of the target sequence fragment, (g) contacting the adaptered fragment with an enzyme that removes the incorporated uracils, thereby creating at least one interval flanked by an exposed 3-prime terminus and an exposed 5-prime terminus of the adaptered fragment, (h) ligating an internal branch adapter to the exposed 3-prime terminus in the at least one interval and ligating an L-adapter to the exposed 5-prime terminus in the interval, and (i) joining the internal branch adapter that has been ligated to the exposed 3-prime terminus and the L-adapter that has been ligated to the exposed 5-prime terminus in step (h), thereby creating a shortened adaptered fragment, thereby producing a set of shortened adaptered fragments comprising shortened target sequence fragments having sequences that correspond to different regions of the target sequence and the different regions are overlapping.

Embodiment 42 is the method of Embodiment 41, wherein ligating the internal branch adapter and the L-adapter comprises contacting the internal branch adapter and the L-adapter with an splint oligonucleotide, wherein the a splint oligonucleotide comprises a 5-prime portion that is complementary to a sequence in the internal branch adapter and a 3-prime portion that is complementary to the L-adapter, thereby the splint oligonucleotide hybridizes to the internal branch adapter via the 5-prime portion and the splint oligonucleotide hybridizes to the L-adapter via the 3-prime portion, thereby ligating the internal branch adapter and the L-adapter.

Embodiment 43 is a method for preparing a plurality of sets of adaptered fragments, wherein each adaptered fragment is a single-stranded nucleic acid comprising a target sequence fragment having a first end and a second end, a 5-prime adapter sequence, a 3-prime adapter sequence, a primer-binding sequence, and a complement of a barcode sequence, wherein the method comprises (a) providing a barcoded fragment comprising a barcode sequence, a target sequence, and a primer binding sequence, wherein the barcoded fragment is immobilized on a bead at one terminus, (b) annealing a primer comprising the 5-prime adapter sequence to the primer-binding sequence in the barcoded fragment, wherein the 5-prime adapter sequence comprises i) a complement of the barcode sequence, and ii) a primer sequence complementary to the primer binding sequence in the barcoded fragment, (c) extending the primer to produce an extended primer comprising a target sequence fragment and the complement of the barcode sequence, (d) contacting the extended primer with a first branch adapter comprising a 3-prime portion comprising a degenerate sequence region, thereby forming a first extension product comprising the degenerate sequence region at the 3-prime portion, wherein the 3-prime portion is hybridized to the barcoded fragment through the degenerate sequence region, (e) extending the 3-prime portion of the first extension product to generate a second extension product, and (f) contacting the second extension product with a second branch adapter to produce the adaptered fragment.

Embodiment 44 is the method of Embodiment 43, wherein the method further comprises (g) denaturing to separate the adaptered fragment from the barcoded fragment.

Embodiment 45 is the method of Embodiment 44, wherein the method further comprises repeating steps (b)-(g) for one or more cycles under extension-controlling conditions to produce one or more adaptered fragments.

Embodiment 46 is a DNA complex comprising a plurality of fragments hybridized to one or more monomers of a DNA concatemer, wherein the plurality of fragments are separated by intervals, wherein each of the plurality of fragments comprises a barcode sequence and a target sequence fragment having a first end and a second end, wherein the target sequence fragments of the plurality of fragments have identical nucleotide sequences at the first end and differ from each other by truncations at the second end, such that the target sequence fragments of the plurality of fragments have different length.

Embodiment 47 is the DNA complex of Embodiment 46, wherein each of the plurality of fragments is ligated to an L-adapter at 5-prime terminus and a branch adapter at 3-prime terminus.

Embodiment 48 is a DNA complex comprising (a) a barcoded fragment immobilized on a solid support, wherein the barcoded fragment comprises a barcode sequence and a target sequence, and (b) a polynucleotide hybridized to the barcoded fragment, wherein the polynucleotide comprises a 5-prime portion comprising a complement of the barcode sequence, a 3-prime portion comprising a target sequence fragment, wherein the 5-prime portion and the 3-prime portion are annealed to the barcoded fragment, leaving a middle portion not annealed to the barcoded fragment, thereby forming a bubble.

Embodiment 49 is a plurality of DNA complexes of any one of Embodiments 46-48, wherein the DNA complexes share the same barcode sequence.

Embodiment 50 is a composition comprising a nested set of adaptered fragments each comprising a barcode sequence and a target sequence fragment having a first end and a second end, a 5-prime adapter sequence, and a 3-prime adapter sequence, wherein the target sequence fragments have identical nucleotide sequences at the first end and differ from each other by truncations at the second end, such that the nested set of adaptered fragments comprises a plurality of target sequence fragments having different length, and wherein the nested set of adaptered fragments share same barcode sequence.

Embodiment 2.1. A method of producing single-stranded DNA circles comprising single-stranded adaptered constructs for sequencing comprising:
preparing a plurality of nested sets of single-stranded nucleic acid constructs, wherein each single-stranded nucleic acid construct in each nested set comprises a target sequence portion flanked by a first adapter sequence and a second adapter sequence, wherein the first adapter sequence comprises a barcode sequence and a primer-binding sequence,
wherein each target sequence portion has a first end and a second end,
wherein the distance between the first end and the barcode sequence is shorter than the distance between the second end and the barcode sequence,
wherein the single-stranded nucleic acid constructs in each nested set share the same barcode sequence and the single-stranded nucleic acid constructs in different nested sets have different barcode sequences,
wherein for each nested set of single-stranded nucleic acid constructs,
(a) the target sequence portions in that nested set have identical nucleotide sequences near the first ends and differ from each other by truncations near the second ends, such that each nested set of single-stranded nucleic acid constructs comprises a plurality of target sequence portions having different lengths, and
(b) circularizing the single-stranded nucleic acid constructs in each nested set to produce the single-stranded DNA circles, in which the first adapter sequence and the second adapter sequence are joined.

Embodiment 2.2 The method of embodiment 2.1, wherein each nested set of single-stranded nucleic acid constructs is prepared by:
(i) preparing adaptered double-stranded genomic fragments each comprising a target sequence flanked by the first adapter sequence and a third adapter sequence,
(ii) amplifying the adaptered double-stranded genomic fragments to produce amplified genomic fragments by using primers hybridized to the first and third adapter sequences,
(iii) contacting the amplified genomic fragments from (ii) with a nicking agent to produce nicks in the target sequences in one strand of the amplified genomic fragments,
(iv) ligating a second adapter comprising the second adapter sequence at the nicks in (iii) via branch ligation to form ligated products, and
(v) denaturing the ligated products from (iv) to form the single-stranded nucleic acid constructs, each comprising the first adapter sequence and the second adapter sequence.

Embodiment 2.3. The method of embodiment 2.1, wherein each nested set of single-stranded nucleic acid constructs is prepared by
(i) preparing adaptered double-stranded genomic fragments each comprising a target sequence flanked by the first adapter sequence and a third adapter sequence,
(ii) amplifying the adaptered double-stranded genomic fragments to produce amplified genomic fragments,
(iii) distributing the amplified genomic fragments into a plurality of aliquots,
(iv) denaturing the amplified genomic fragments in (iii) to prepare single-stranded genomic fragment, wherein at least some of the single-stranded genomic each comprising the primer-binding sequence,
(iv) extending a primer hybridized to the primer-binding sequence under extension-controlling conditions such that the lengths of extension products from different aliquots are different, thereby producing extension products having newly formed ends, and the extension products have different sequences near the newly formed ends in different aliquots,
wherein each extension product comprises a target sequence portion, and
(v) ligating a second adapter comprising the second adapter sequence at the newly formed ends via branch ligation in each aliquot, thereby producing the single-stranded nucleic acid constructs, each comprising the first adapter sequence and the second adapter sequence.

Embodiment 2.4. The method of embodiment 2.1, wherein each nested set of single-stranded nucleic acid constructs is prepared by
(i) preparing adaptered double-stranded genomic fragments each comprising a target sequence flanked by the first adapter sequence and a third adapter sequence,
(ii) amplifying the adaptered double-stranded genomic fragments to produce amplified genomic fragments,
(iii) distributing the amplified genomic fragments into a plurality of aliquots,
(iv) adding a double-stranded DNA nuclease with 3′→5′ nuclease activity the plurality of aliquots under controlled conditions such that the lengths of products remaining after the digestion in different aliquots are different, thereby producing digestion products having newly formed ends with different sequences in different aliquots,
wherein each digestion product comprises a target sequence portion, and
(v) ligating a second adapter comprising the second adapter sequence at the newly formed ends via branch ligation in each aliquot, thereby producing the single-stranded nucleic acid constructs, each comprising the first adapter sequence and the second adapter sequence.

Embodiment 2.5. The method of embodiment 2.1, wherein each nested set of single-stranded nucleic acid constructs is prepared by
(i) preparing adaptered double-stranded genomic fragments each comprising a target sequence flanked by the first adapter sequence and a third adapter sequence,
(ii) amplifying the adaptered double-stranded genomic fragments to produce amplified genomic fragments,
(iii) denaturing the amplified genomic fragments to prepare single-stranded genomic fragments, wherein at least some of the single-stranded genomic fragments each comprising the primer-binding sequence,
(iv) for each single-stranded genomic fragment,
extending a primer hybridized to the primer-binding sequence for a first period of time to produce an extended primer,
wherein the extension is incomplete such that the length of the extended primer is a fraction of the length of the single-stranded genomic fragment,
wherein the extended primer comprises a target sequence portion, and
ligating a second adapter at the newly formed end of the extended primer via branch ligation, thereby producing single-stranded nucleic acid constructs in one reaction mixture, each comprising the first adapter sequence and the second adapter sequence,
(v) repeat step (iv) for multiple rounds, for each round, the primer is further extended for an additional period of time, and an additional adapter having a unique positional barcode is ligated to the further extended primer,
wherein the additional adapter is used in a molar amount that is a fraction of the total molar amount of the amplified genomic fragments,
thereby producing a mixture of nested set of single-stranded nucleic acid constructs.

Embodiment 2.6. The method of embodiment 2.3, wherein the target sequence comprises repetitive sequences, wherein the second adapter comprises a positional barcode sequence that is unique to each aliquot,
wherein the single-stranded nucleic acid constructs formed in (v) in different aliquots comprise different positional barcode sequence, and the single-stranded nucleic acid constructs in the same aliquot share the same positional barcode sequence.

Embodiment 2.7. The method of embodiment 2.1, wherein the primer-binding sequence is 3-prime in relation to the barcode sequence.

Embodiment 2.8. The method of embodiment 2.1, wherein the method further comprises
(vi) fragmenting the single-stranded DNA circles to produce a plurality of single-stranded DNA fragments, wherein at least some of which comprise the barcode sequence,
(vii) producing double-stranded DNA fragments from the single-stranded DNA fragments from step (vi),
(vii) ligating a second adapter to each of the double-stranded DNA fragments from step (vii), thereby producing double-stranded adaptered fragments.

Embodiment 2.9. The method of embodiment 2.8, the method further comprises (viii) amplifying the double-stranded adaptered fragments, and
optionally (ix) selecting the amplified double-stranded adaptered fragments having lengths within a range of 300-1000 bases.

Embodiment 2.10. The method of embodiment 2.1, wherein the method further comprises
(vi) hybridizing a primer to the primer-binding sequence in each of the single-stranded DNA circles,
(vii) extending the primer under extension-controlling conditions using each of the single-stranded DNA circles as templates,
wherein the extending produces an extended primer hybridized to single-stranded DNA circles, thereby producing a plurality of extended primers having different lengths,
wherein said each of the extended primers comprises the barcode sequence and the primer-binding sequence,
(viii) ligating a second adapter to the plurality of extended primers via branch ligation to produce adaptered extended primers.

Embodiment 2.11. The method of embodiment 2.10, wherein the method further comprises
amplifying the adaptered extended primers to produce amplified double-stranded fragments,
selecting the amplified double-stranded fragments having lengths within a range from 300 bases to 1000 bases (will disclosed in the specification nested ranges around the optimal length of 600 bases), and
sequencing the selected amplified double-stranded adaptered fragments.

Embodiment 2.12. The method of embodiments 2.1-2.11, wherein the single-stranded DNA circles are prepared in solution, without solid supports.

Embodiment 2.13. The method of embodiment 2.1, wherein the first end or the second end is attached to a solid support.

Embodiment 2.14. A method of producing double-stranded adaptered constructs for sequencing, wherein the method comprises:
(i) amplifying a plurality of genomic fragments, each genomic fragment comprising a target sequence, to produce a plurality sets of amplified nucleic acid fragments in a mixture, wherein the amplified nucleic acid fragments in each nested set share the same target sequence, optionally the amplification is performed using target-specific primers,
for each nested set, the method further comprises
(ii) contacting the amplified nucleic acid fragments with an enzyme, wherein the enzyme introduces breaks in the amplified nucleic acid fragments,
(iii) distributing the mixture of fragments into a plurality of aliquots,
(iv) performing nick translation on the aliquots of fragments to synthesize DNA strands under conditions such that the DNA strands synthesized in different aliquots have different lengths, wherein each of the DNA strands comprises a target sequence portion with a first end and a second end, and wherein the DNA strands in different aliquots share the same sequence near the first ends and have different sequence near the second ends,
(v) for each aliquot, ligating second adapters to the second ends of the DNA strands synthesized in (iv) via branch ligation, wherein each second adapter is a partially double stranded adapter comprising a first adapter oligonucleotide and a second adapter oligonucleotide,
wherein both the first adapter oligonucleotide and a second adapter oligonucleotide are complementary and hybridized to each other,
wherein each of the second adapters comprises a positional barcode sequence,
wherein each ligation comprises joining a 5-prime end of the first adapter oligonucleotide of the second adapter to a second end of the synthesized DNA strand,
wherein the first adapter oligonucleotides ligated to the second ends of the synthesized DNA strands in different aliquots comprise different positional barcode sequence, and the first adapter oligonucleotides ligated to the second ends of the synthesized DNA strands in the same aliquot share the same positional barcode sequence,
(vi) combining the synthesized DNA strands ligated with the second adapters from different aliquots from (v) in a single mixture,
(vii) extending a primer hybridized to the first adapter oligonucleotides that have been ligated to the synthesized DNA strands to produce double-stranded fragments having blunt ends, and
(viii) optionally selecting the double-stranded fragments of (vii) with a size within a range from 200 bp-1.5 kb (will disclose a range around the optimal 500-1000 bp in the specification) from the single mixture, and
(ix) ligating a third adapter to the blunt ends of the double-stranded fragments, thereby producing double-stranded adaptered constructs.

Embodiment 2.15. The method of embodiment 2.14, wherein step (i) comprises amplifying the plurality of genomic fragments in a mixture comprising uracils, thereby producing amplified nucleic acid fragments with uracils incorporated, and
wherein step (ii) comprises contacting the amplified nucleic acid fragments with a uracil-DNA glycosylase, wherein the uracil-DNA glycosylase removes the uracils from the amplified genomic fragments.

Embodiment 2.16. The method of embodiment 2.14, wherein the amplifying the plurality of genomic fragments in step (i) is performed using primers comprising the uracils, thereby producing the plurality sets of amplified nucleic acid fragments comprising uracil.

Embodiment 2.17. The method of embodiment 2.16, wherein each of the plurality of genomic fragments is amplified using a forward primer and a reverse primer, and wherein each forward primer comprise one or more uracils.

Embodiment 2.18. The method of embodiment 2.17, wherein each of the plurality of genomic fragments is amplified using a forward primer and a reverse primer, and wherein each reverse primer comprise a single uracil.

Embodiment 2.19. The method of embodiment 2.14, wherein step (ii) comprises contacting the amplified genomic fragments with an endonuclease, wherein the endonuclease cuts the amplified genomic fragments at random.

Embodiment 2.20. The method of embodiment 2.19, wherein the endonuclease is EndoIV or APE1.

Embodiment 2.21. A reaction mixture comprising the single-stranded DNA circles produced in embodiment 2.7.

Embodiment 2.22. A reaction mixture comprising the combined synthesized DNA strands from step (vi) of the embodiment 1.19.

### EXAMPLES

### 1. Example 1. In Solution Co-Barcoding Using Infrequent Random Nicking

This example describes generating full coverage of a 1-20 kb DNA molecule. It can be a useful method for assembly for most sequences especially using sequencing platforms such as SE400-SE1000 or PE300+ MPS reads. Only when the target nucleic acid comprise highly repetitive sequence, will positional co-barcoding, as described herein, be needed. The method starts by ligating a barcoded adapter on one end of a molecule and a nonbarcoded adapter on the opposite end, this is achieved through ligation of a Y adapter or other commonly used methods. This method can also be used for targeted sequences if a common adapter tag is added to each PCR primer with one of the adapter tags in each PCR primer pair containing a barcode. After PCR, molecules are treated with a nonspecific nicking enzyme at low concentration, low temperature, and/or a short period of time to introduce a nick within each template. If necessary, this nick can be widened into a gap of several bases in the sequence using 5′ or 3′ exonucleases or polymerases without dNTPs. Branch ligation is then performed to add another adapter. The molecules are then circularized using a splint oligonucleotide between the branch ligation adapter and the barcode containing adapter. FIG. 3A. The circles are then fragmented to 500-1000 base pairs followed by primer extension from the barcoded adapter in such a way that the barcode is copied. One more round of ligation of an adapter and the molecules can now be sequenced directly or PCR amplified and sequenced.

Another embodiment of this process uses controlled extension of ˜600 bases, after circularization, followed by 3′ branch ligation and then PCR. FIG. 4B. This has the benefit of generating products that fall within a relatively narrow size range as opposed to random fragmenting that will generate a broad distribution of sizes. Any short artifact products are removed through exonuclease treatment or purification.

The final result of this process is a series of overlapping sequence reads from each original DNA molecule that all share the same barcode sequence. Random nicking provides similar coverage of short and long DNA molecules present in the same pool. This enables complete reassembly of each original DNA molecule.

### 2. Example 2. In Solution Positional Co-Barcoding of 1-20 kb Fragments

This example is similar to Example 1 except that it incorporates a barcode that can be shared amongst all the sub-fragments of the original molecule (co-barcoding). The process starts by either using targeted PCR primers containing a common adapter tag with a random barcode to amplify specific regions or by ligation of an adapter with common sequence and a random barcode to dsDNA fragments 1-20 kb in length. Preferably a pool has DNA fragments of similar length. For pools having long and short DNA, specific methods can be used to minimize over coverage of shorter fragments. The products are PCR amplified and then split into 10-20 pools followed by a different amount of controlled extension or ExoIII digestion per pool (as described above) or controlled nick translation. Short DNA fragments will be completely extended to form blunt ends, and these fragments with blunt ends can be blocked from branch ligation using methods known in the art, for example, DNA tailing or 3′ blocking by terminal transferase. After this step, a 3′ branch ligation is performed to add an adapter with a common sequence and a barcode sequence specific for each pool (positional barcode). Now, unlike above, the products are circularized, this links the DNA molecule barcode to the positional barcode with some common adapter sequence in between. Next the circles are fragmented to 500-1000 base pairs and the fragments are primer extended with a primer the is to the 5′ of both the molecule and the positional barcode. After extension, a third adapter is ligated and now sequencing, or PCR and sequencing can be performed. Instead of primer extension a blunt-end third adapter with non-phosphorylated 5′ end can be ligated followed by PCR and sequencing. FIG. 3B.

Another method that can be employed based on this process, is to allow the reactions above to occur in a single tube, as opposed to in separate pools. This can be achieved by adding a limited amount of 3′ branch ligation adapter with a different sequence after each time interval. For example, a first 3′ branch ligation adapter is added 10 minutes after the initiation of the extension, a second 3′ branch ligation adapter is added 20 minutes after the initiation of the extension, and so no, and the first and the second 3′ branch ligation adapter have different sequences. The ideal amount of 3′ branch ligation adapter is one that would result in the ligation of 1-10% of the total number of molecules (depending on the length of the molecules used). This process of adding a limited amount of adapter would be repeated 10-20 or more times (equivalent to the number of total pools used in the other approach). This has the advantage of being performed in a single tube but will require multiple rounds of adapter pipetting into the same tube.

### 3. Example 3. Target Enriched 2-20 kb Pools for Non-Related Sequences

The methods described in this example performed on using long range PCR on targeted regions of the genome. In some cases, multiplex PCRs can be performed such that 100s to 1000s of different target regions can be amplified in one or more reactions. After amplification, the products are split into different pools. Depending on the size of amplicons and the sequencing read lengths expected to be used, the number of pools can be increased or decreased, but around 10-20 pools is a good number for a 5 kb product with ˜500 bases of reads (either pair end 250 or single end 500). For each pool either a timed digestion with ExoIII or a controlled extension with a polymerase with 5′-3′ exo activity is performed (e.g., E. coli DNA polymerase 1). Importantly, for each pool the time is varied in steps for ExoIII treatment (e.g., 1 minute, 2 minutes, 3 minutes, 4 minutes, etc.) in such a way that the amount digested between each pool is roughly 500 bases in this example. Likewise, if controlled extension or nick translation is performed, the time or ratio of dNTPs can be varied to achieve similar results. It is important to note that there is variability in the amount of extension or digestion in each pool, instead of a specific length of product, there is a range of products. This results in overlapping fragments between the different pools and after sequencing this overlap will make in silico assembly of each original molecule much easier. After this step, a 3′ branch ligation is performed to add an adapter with a common sequence and a barcode sequence specific for each pool. This adapter can include a biotin on the 3′ end to help with purification steps. The pools are then combined, and the products are fragmented to ˜500-1000 base pairs followed by primer extension from the adapter sequence and then ligation of a second common adapter. The products can then either be PCR amplified or directly circularized for DNB formation and sequencing. FIG. 5.

### 4. Example 4. Looping-Extension Complete stLFR

The loop-mediated complete stLFR involves ligating two functionalized partially double stranded blunt-end adapters to the end-repaired DNA fragments bearing 5′-phosphate groups.

### Ligation of Blunt-Ended Adapters to the End-Repaired DNA Fragments Bearing 5′-Phosphate Groups

The first partially double stranded blunt-end adapter (for example, AD153UMI_5, FIG. 13A) has a longer strand (1313) and a shorter strand (1314) annealed to form a blunt end and an unpaired end. The blunt end is ligatable. The longer strand (1313) comprises a single-stranded 5′-overhang, which comprises one or more barcodes (UID) (1319), such as a unique molecular identification sequences (UMI) or a multiplex sample barcode (1319). The single-stranded 5′-overhang may also comprise a sequence complementary to the universal amplification primer. The barcode sequences may be present as a single sequence or as several separate sequences. Optionally, this single-stranded 5′-overhang comprises a single T overhang at the 3′ for the ligation to the A-tailed DNA fragment. The shorter strand (1314), annealed to the longer strand (1313), comprises a 5′-end that does not contain a 5′-phosphate group and thus unligatable to the DNA fragment. The 3′-end of the shorter strand (1314) is also unligatable because it has been modified to prevents ligation. These modifications of the 3′ for ligation prevention include but are not limited to an inverted nucleoside, a dideoxy nucleoside, 3′-amino group, 3′-phosphate group. When the first partially double stranded blunt end adapter contacts a genomic DNA (1310) under ligation conditions, only the longer strand (1313) is ligated to the gDNA, while the shorter strand (1314), neither the 5′ nor the 3′ end of which is ligatable, cannot be ligated to the genomic DNA fragment, leaving a nick (1317) between the shorter strand and the gDNA strand. See FIG. 13A.

The second partially double stranded blunt-end adapter (e.g., Ad183 as shown in FIG. 13) is designed similar to the first one with the exception that it does not comprise barcode sequences (UID).

Genomic fragments are then ligated with the adapters above and purified using SPRI bead purification (Beckman Coulter Life Sciences, Indianapolis, IN). The adapter-ligated DNA molecules are subjected to enzymatic extension of 3′-ends of genomic DNA fragments to the unligatable 5′-end of the shorter strand (1314) of the partially double-stranded adaptera using DNA polymerases possessing a strand-displacing activity (e.g., Bst DNA polymerase, Large fragment; Phi29 DNA polymerase; Bsu DNA polymerase, Large fragment; Bsm DNA polymerase, Large Fragment) or possessing 5′-3′ exonuclease activity (e.g., rTaq DNA polymerase, E. coli DNA polymerase I). This forms double-stranded adapter-ligated DNA molecules with double-stranded adapters attached to the DNA fragment sequence (1318). The 3′-end of shorter strand (1320.2) of the branch adapter comprises 15-20 bases sequences complementary to the 5′-end of the longer strand of the branch adapter. The longer strand of the branch adapter comprises barcode sequences and to have melting temperatures (Tm) of 50°-70° C. The 3′-end of the short stand (1320.2) is blocked by 3′-terminal modifications preventing a ligation (e.g., dideoxy nucleoside, 3′-amino group, 3′-phosphate group). FIG. 13B. Adapter sequences derived from the first partially double-stranded adapter comprise sites for universal amplification primers, therefore the double-stranded adapter-ligated DNA molecules (1318) can be amplified by PCR or other amplification method which rely on two priming sequences. FIG. 13A.

The random nicking and gapping is performed. This can be achieved by using a non-specific nicking nuclease, which only breaks the DNA backbone of one strand per catalysis; for examples Vvn and mutants, Shrimp dsDNA specific endonuclease, DNAse I. This can also be achieved by using mixtures of multiple nicking enzymes such as several site-specific nickases (e.g., CCD). In some cases, an additional enzyme with 3′ exo activity (such as DNA Polymerase I, Klenow Fragment without nucleotides, Exonuclease III, or similar) or with 5′ exo activity (Bst DNA polymerase full length without nucleotides, T7 exonuclease, Exonuclease VIII truncated, Lambda exonuclease, T5 exonculease, or similar) can be added as well to increase the opening of the nick for more room for branch adapter ligation. Low processivity exonuclease are preferred to open a short gap (e.g., 2-7 bases) and disassociate from DNA to allow adapter ligation. FIGS. 13A and 13B. Random nicking produced a set of fragments having different length of the target sequence fragments and share the common barcode sequence at the 5′. One of such fragments is shown as 1341 in FIG. 13B

A branch adapter (e.g., AD153UMI_5R, shown in FIG. 13B) is then ligated to the 3′-side of nicks or ssDNA gaps in the adapter-ligated DNA fragments in the presence of a T4 DNA ligase. The branch adapter (1320) is a partially double stranded DNA adapter molecule with a 5′-Phosphate on the longer strand (1320.1). This produced a set of fragments having different length of the target sequence fragments, each flanked by adapter sequence AD153UMI_5 and AD153UMI_R, one of which is shown as 1342 in FIG. 13B.

The longer strand of the first partially double stranded blunt-end adapter and the longer strand of the branch adapter comprise a first hybridization sequence (1432) and a second hybridization sequence (1433), respectively. The first hybridization sequence (1432) is located 3′ relative to the barcode sequence (1319).

In the case where an exonuclease is used to open gaps as described above, if necessary, protection of the DNA adapters can be achieved through phosphorothioated bonds between bases and/or modified bases at the 5′ and 3′ ends of the adapters.

The ligation of the branch adapter to the adaptered DNA fragments (e.g., adaptered genomic DNA fragments) can be performed in solution or on beads. In the case where the process is performed on the beads surface, adaptered DNA fragments are preloaded to beads at high concentration of PEG (5%-15%) before adding other reaction components.

In some embodiments, the branch ligation reaction is performed in the presence of additives (e.g., polyethylene glycol or betaine) to increase the activity of ligation and/or the nicking enzyme. This reaction can be incubated at room temperature, 37 C, or cycled between various temperatures, such as 5-15 C degrees and 37 C degrees at a pH ranging from 5.0 to 9.0. After 5 minutes to several hours. The amount of time and nickase concertation varies depending on the desired number of nicks per DNA fragment. The reaction can be stopped through a DNA purification method (such as Ampure XP beads) if performed in solution, or simply through a washing step with a Tris NaCl buffer containing PEG (5%-15%) if performed on beads.

In some embodiments, after the branch ligation, the DNA fragments are denatured. subjecting the reaction mixture to heat to a temperature between 90° C.-95° C., end points inclusive. Alternatively, branch-ligated DNA fragments can be denatured by alkaline agents (e.g., 0.05M-0.2M NaOH or KOH) with further neutralization by neutralizing agents (e.g., HCl, Tris-HCl, MOPS). Single stranded DNA molecules (1343) comprising genomic DNA and the adapter sequences at both ends are formed. FIG. 13B.

Alternatively, in some embodiments, instead of denaturing, a 5′ single-stranded tail (e.g., 1432 in FIG. 13 or FIG. 14) at adapter-ligated DNA fragments required for the hybridization of the 3-end of branch adapter can be generated by digestion using one or more dsDNA specific exonucleases possessing 3′-5′ exonuclease activity (e.g., Exonuclease III).

### DNA Loop Formation and Enzymatic Extension of the Hybridized 3′-End of Branch Adapter

The longer strand of the branch adapter and the longer strand of the first partially double stranded blunt-end adapter comprise complementary sequences (1432 and 1433) and are capable of hybridizing to each other. Hybridization is carried out in a hybridization buffer containing buffering agents (e.g., Tris-HCl, MOPS, sodium phosphate), salts, and co-factors which are essential for subsequent enzymatic reactions, such as MgCl2, dNTPs. FIG. 14.

The DNA hybridization step is followed by the linear extension step of the hybridized 3′-end of branch adapter (e.g., AD153UMI_5R shown in FIG. 14) to copy the barcode on the first adapter AD153 UMI_5. To increase the specificity, linear extension is performed using DNA polymerases lacking 3′-exonuclease activity, such as Taq DNA polymerase, Klenow Fragment (3′→5′ exo-), Bst DNA Polymerase, Large Fragment. Depending on used DNA polymerase, the extension can be carried out at different temperatures ranging from 30° C. to 75° C.

The product (1431) of linear extension represents partially duplex DNA molecules with double-stranded adapter comprising UID sequence attached to the DNA fragment. The product is then denatured to form a single-stranded sequence with adapter sequences at both ends (1441), which brings the ends of the target sequence fragments 1411, 1421, 1431, etc. close to the barcode sequence 1319. Adapter sequence comprises barcode sequences and the site for universal amplification primer, therefore, can be used in the next step, controlled primer extension to produce fragments having lengths that are suitable for sequencing.

While this invention has been disclosed with reference to specific aspects and embodiments, it is apparent that other embodiments and variations of this invention may be devised by others skilled in the art without departing from the true spirit and scope of the invention.

Each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

1. A method of producing single-stranded adaptered constructs for sequencing comprising:
preparing a plurality of nested sets of single-stranded nucleic acid constructs, wherein each single-stranded nucleic acid construct in each nested set comprises a target sequence portion flanked by a first adapter sequence at the 5′ end and a second adapter sequence at the 3′ end,
wherein the first adapter sequence comprises, from 5′ to 3′, a primer-binding sequence, a barcode sequence and a first hybridization sequence and the second adapter sequence comprises a second hybridization sequence,
wherein the first and the second hybridization sequences are complementary to each other,
wherein each target sequence portion has a first end and a second end,
wherein the distance between the first end and the barcode sequence is shorter than the distance between the second end and the barcode sequence,
wherein the single-stranded nucleic acid constructs in each nested set share the same barcode sequence and the single-stranded nucleic acid constructs in different nested sets have different barcode sequences, and
wherein for each nested set of single-stranded nucleic acid constructs, the target sequence portions in that nested set have identical nucleotide sequences near the first ends and differ from each other by truncations near the second ends, such that each nested set of single-stranded nucleic acid constructs comprises a plurality of target sequence portions having different lengths.

2. The method of claim 1, further comprising subjecting the plurality of nested sets of single-stranded nucleic acid constructs to hybridization conditions, whereby the first adapter sequence is hybridized to the second adapter sequence, thereby forming a loop.

3. The method of claim 2, wherein the method further comprises extending the second adapter sequence to copy the barcode sequence and the primer-binding sequence in the first adapter sequence using a DNA polymerase to form an extension product.

4. The method of claim 3, wherein the method further comprises denaturing the extension product to open the loop, thereby forming linear single-stranded DNA constructs, wherein each linear single-stranded DNA construct comprises a barcode sequence and primer-binding sequence, wherein the primer-binding sequence is located 3′ relative to the barcode sequence.

5. The method of claim 4, wherein the method further comprises annealing a primer to the primer-binding sequence at the 3′ of the linear single-stranded DNA construct and extending the primer to generate an extension product having a length that is suitable for sequencing.

6. A method of producing single-stranded DNA circles comprising single-stranded adaptered constructs for sequencing comprising:
preparing a plurality of nested sets of single-stranded nucleic acid constructs, wherein each single-stranded nucleic acid construct in each nested set comprises a target sequence portion flanked by a first adapter sequence and a second adapter sequence, wherein the first adapter sequence comprises a barcode sequence and a primer-binding sequence,
wherein each target sequence portion has a first end and a second end,
wherein the distance between the first end and the barcode sequence is shorter than the distance between the second end and the barcode sequence,
wherein the single-stranded nucleic acid constructs in each nested set share the same barcode sequence and the single-stranded nucleic acid constructs in different nested sets have different barcode sequences,
wherein for each nested set of single-stranded nucleic acid constructs,
(a) the target sequence portions in that nested set have identical nucleotide sequences near the first ends and differ from each other by truncations near the second ends, such that each nested set of single-stranded nucleic acid constructs comprises a plurality of target sequence portions having different lengths, and
(b) circularizing the single-stranded nucleic acid constructs in each nested set to produce the single-stranded DNA circles, in which the first adapter sequence and the second adapter sequence are joined.

7. The method of any one of claims 1-6, wherein each nested set of single-stranded nucleic acid constructs is prepared by:
(i) preparing adaptered double-stranded genomic fragments each comprising a target sequence flanked by the first adapter sequence and a third adapter sequence,
(ii) amplifying the adaptered double-stranded genomic fragments to produce amplified genomic fragments by using primers hybridized to the first and third adapter sequences,
(iii) contacting the amplified genomic fragments from (ii) with a nicking agent to produce nicks in the target sequences in one strand of the amplified genomic fragments,
(iv) ligating a second adapter comprising the second adapter sequence at the nicks in (iii) via branch ligation to form ligated products, and
(v) denaturing the ligated products from (iv) to form the single-stranded nucleic acid constructs, each comprising the first adapter sequence and the second adapter sequence.

8. The method of any one of claims 1-6, wherein each nested set of single-stranded nucleic acid constructs is prepared by
(i) preparing adaptered double-stranded genomic fragments each comprising a target sequence flanked by the first adapter sequence and a third adapter sequence,
(ii) amplifying the adaptered double-stranded genomic fragments to produce amplified genomic fragments,
(iii) distributing the amplified genomic fragments into a plurality of aliquots,
(iv) denaturing the amplified genomic fragments in (iii) to prepare single-stranded genomic fragment, wherein at least some of the single-stranded genomic each comprising the primer-binding sequence,
(iv) extending a primer hybridized to the primer-binding sequence under extension-controlling conditions such that the lengths of extension products from different aliquots are different, thereby producing extension products having newly formed ends, and the extension products have different sequences near the newly formed ends in different aliquots,
wherein each extension product comprises a target sequence portion, and
(v) ligating a second adapter comprising the second adapter sequence at the newly formed ends via branch ligation in each aliquot, thereby producing the single-stranded nucleic acid constructs, each comprising the first adapter sequence and the second adapter sequence.

9. The method of any one of claims 1-6, wherein each nested set of single-stranded nucleic acid constructs is prepared by
(i) preparing adaptered double-stranded genomic fragments each comprising a target sequence flanked by the first adapter sequence and a third adapter sequence,
(ii) amplifying the adaptered double-stranded genomic fragments to produce amplified genomic fragments,
(iii) distributing the amplified genomic fragments into a plurality of aliquots,
(iv) adding a double-stranded DNA nuclease with 3′→5′ nuclease activity the plurality of aliquots under controlled conditions such that the lengths of products remaining after the double-stranded DNA nuclease digestion in different aliquots are different, thereby producing digestion products having newly formed ends with different sequences in different aliquots,
wherein each digestion product comprises a target sequence portion, and
(v) ligating a second adapter comprising the second adapter sequence at the newly formed ends via branch ligation in each aliquot, thereby producing the single-stranded nucleic acid constructs, each comprising the first adapter sequence and the second adapter sequence.

10. The method of any one of claims 1-6, wherein each nested set of single-stranded nucleic acid constructs is prepared by
(i) preparing adaptered double-stranded genomic fragments each comprising a target sequence flanked by the first adapter sequence and a third adapter sequence,
(ii) amplifying the adaptered double-stranded genomic fragments to produce amplified genomic fragments,
(iii) denaturing the amplified genomic fragments to prepare single-stranded genomic fragments, wherein at least some of the single-stranded genomic fragments each comprising the primer-binding sequence,
(iv) for each single-stranded genomic fragment,
extending a primer hybridized to the primer-binding sequence for a first period of time to produce an extended primer,
wherein the extension is incomplete such that the length of the extended primer is a fraction of the length of the single-stranded genomic fragment,
wherein the extended primer comprises a target sequence portion, and ligating a second adapter via branch ligation to the end of the extended primer formed by the extension,
thereby producing single-stranded nucleic acid constructs in one reaction mixture, each comprising the first adapter sequence and the second adapter sequence,
(v) repeat step (iv) for multiple rounds, for each round, the primer is further extended for an additional period of time, and an additional adapter having a unique positional barcode is ligated to the further extended primer,
wherein the additional adapter is used in a molar amount that is a fraction of the total molar amount of the amplified genomic fragments, thereby producing a mixture of nested set of single-stranded nucleic acid constructs.

11. The method of claim 8, wherein the target sequence comprises repetitive sequences, wherein the second adapter comprises a positional barcode sequence that is unique to each aliquot,
wherein the single-stranded nucleic acid constructs formed in (v) in different aliquots comprise different positional barcode sequence, and the single-stranded nucleic acid constructs in the same aliquot share the same positional barcode sequence.

12. The method of claim 6, wherein the primer-binding sequence is 3-prime in relation to the barcode sequence.

13. The method of claim 6, wherein the method further comprises
(vi) fragmenting the single-stranded DNA circles to produce a plurality of single-stranded DNA fragments, wherein at least some of which comprise the barcode sequence,
(vii) producing double-stranded DNA fragments from the single-stranded DNA fragments from step (vi),
(vii) ligating a second adapter to each of the double-stranded DNA fragments from step (vii), thereby producing double-stranded adaptered fragments.

14. The method of claim 13, the method further comprises (viii) amplifying the double-stranded adaptered fragments, and
optionally (ix) selecting the amplified double-stranded adaptered fragments having lengths within a range of 300-1000 bases.

15. The method of claim 6, wherein the method further comprises
(vi) hybridizing a primer to the primer-binding sequence in each of the single-stranded DNA circles,
(vii) extending the primer under extension-controlling conditions using each of the single-stranded DNA circles as templates,
wherein the extending produces an extended primer hybridized to single-stranded DNA circles, thereby producing a plurality of extended primers having different lengths,
wherein said each of the extended primers comprises the barcode sequence and the primer-binding sequence,
(viii) ligating a second adapter to the plurality of extended primers via branch ligation to produce adaptered extended primers.

16. The method of any one of claims 6-15, wherein the method further comprises
amplifying the adaptered extended primers to produce amplified double-stranded fragments,
selecting the amplified double-stranded fragments having lengths within a range from 300 bases to 1000 bases (will disclosed in the specification nested ranges around the optimal length of 600 bases), and
sequencing the selected amplified double-stranded adaptered fragments.

17. The method of claims 1-16, wherein the single-stranded DNA circles are prepared in solution, without solid supports.

18. The method of claim 6, wherein the first end or the second end is attached to a solid support.

19. A method of producing double-stranded adaptered constructs for sequencing, wherein the method comprises:
(i) amplifying a plurality of genomic fragments, each genomic fragment comprising a target sequence, to produce a plurality sets of amplified nucleic acid fragments in a mixture, wherein the amplified nucleic acid fragments in each set share the same target sequence, optionally the amplification is performed using target-specific primers,
for each set, the method further comprises
(ii) contacting the amplified nucleic acid fragments with an enzyme,
wherein the enzyme introduces breaks in the amplified nucleic acid fragments,
(iii) distributing the mixture of fragments into a plurality of aliquots,
(iv) performing nick translation on the aliquots of fragments to synthesize DNA strands under conditions such that the DNA strands synthesized in different aliquots have different lengths, wherein each of the DNA strands comprises a target sequence portion with a first end and a second end, and wherein the DNA strands in different aliquots share the same sequence near the first ends and have different sequence near the second ends,
(v) for each aliquot, ligating second adapters to the second ends of the DNA strands synthesized in (iv) via branch ligation, wherein each second adapter is a partially double stranded adapter comprising a first adapter oligonucleotide and a second adapter oligonucleotide,
wherein both the first adapter oligonucleotide and a second adapter oligonucleotide are complementary and hybridized to each other,
wherein each of the second adapters comprises a positional barcode sequence,
wherein each ligation comprises joining a 5-prime end of the first adapter oligonucleotide of the second adapter to a second end of the synthesized DNA strand,
wherein the first adapter oligonucleotides ligated to the second ends of the synthesized DNA strands in different aliquots comprise different positional barcode sequence, and the first adapter oligonucleotides ligated to the second ends of the synthesized DNA strands in the same aliquot share the same positional barcode sequence,
(vi) combining the synthesized DNA strands ligated with the second adapters from different aliquots from (v) in a single mixture,
(vii) extending a primer hybridized to the first adapter oligonucleotides that have been ligated to the synthesized DNA strands to produce double-stranded fragments having blunt ends, and
(viii) optionally selecting the double-stranded fragments of (vii) with a size within a range from 200 bp-1.5 kb from the single mixture, and
(ix) ligating a third adapter to the blunt ends of the double-stranded fragments, thereby producing double-stranded adaptered constructs.

20. The method of claim 19,
wherein step (i) comprises amplifying the plurality of genomic fragments in a mixture comprising uracils, thereby producing amplified nucleic acid fragments with uracils incorporated, and
wherein step (ii) comprises contacting the amplified nucleic acid fragments with a uracil-DNA glycosylase, wherein the uracil-DNA glycosylase removes the uracils from the amplified genomic fragments.

21. The method of claim 19,
wherein the amplifying the plurality of genomic fragments in step (i) is performed using primers comprising uracils, thereby producing the plurality sets of amplified nucleic acid fragments comprising uracil.

22. The method of claim 21, wherein each of the plurality of genomic fragments is amplified using a forward primer and a reverse primer, and
wherein each forward primer comprise one or more uracils.

23. The method of claim 22, wherein each of the plurality of genomic fragments is amplified using a forward primer and a reverse primer, and
wherein each reverse primer comprise a single uracil.

24. The method of claim 19, wherein step (ii) comprises contacting the amplified genomic fragments with an endonuclease, wherein the endonuclease cuts the amplified genomic fragments at random.

25. The method of claim 24, wherein the endonuclease is EndoIV or APE1.

26. A reaction mixture comprising the single-stranded DNA circles produced in claim 6.

27. A reaction mixture comprising the combined synthesized DNA strands from step (vi) of the claim 19.

28. A method for preparing a plurality of nested sets of adaptered fragments, wherein each adaptered fragment is a single-stranded nucleic acid comprising a target sequence fragment having a first end and a second end, a 5-prime adapter sequence, a 3-prime adapter sequence, a primer sequence, and a barcode sequence,
wherein in each nested set of adaptered fragments, the target sequence fragments have identical nucleotide sequences at the first end and differ from each other by truncations at the second end, such that each nested set of adaptered fragments comprises a plurality of target sequence fragments having different length,
wherein the first end is closer to the barcode sequence than the second end,
wherein the method comprises:
(a) providing, in a reaction, a population of single-stranded DNA concatemers, wherein each concatemer comprises a plurality of identical monomers, and each monomer comprises a complement of a target sequence, a complement of the barcode sequence that identifies the concatemer, and a primer-binding sequence shared by the population of single-stranded concatemers,
wherein the primer-binding sequence comprises a sequence that is complementary to the primer sequence,
wherein both the primer-binding sequence and complement of the barcode sequence are 3-prime to the complement of the target sequence;
(b) annealing primers comprising the primer sequence to primer-binding sequences of multiple monomers of each of plurality of the concatemers;
(c) extending at least some of the primers hybridized to the primer-binding sequences with a DNA polymerase that has 5′-->3′ exonuclease activity and does not have strand displacement activity,
wherein the extending produces a plurality of extended primers, each said extended primer comprising a target sequence fragment with barcode sequences and primer sequences,
wherein the extended primers are hybridized to the concatemer;
wherein the extended primers are separated by intervals, and
(d) contacting the plurality of the extended primers with
a 5-prime adapter comprising the 5-prime adapter sequence,
a 3-prime adapter comprising the 3-prime adapter sequence,
a DNA ligase, and
an exonuclease having single-strand DNA exonuclease activity under conditions in which the exonuclease degrades a portion of the target sequence fragments in the extended primers, to produce shortened extended primers, the 5-prime adapters are ligated to the 5′ end of the shortened extended primers, and the 3-prime adapters are ligated to the 3′ end of the shortened extended primers,
thereby producing a group of plurality of nested sets of adaptered fragments.

29. The method of claim 28, wherein the population of single-stranded DNA concatemers are produced by rolling circle replication of circular templates, wherein each of the circular templates comprises the target sequence, the barcode sequence and the primer sequence.

30. The method of claim 28, wherein the 5-prime adapter is an L-adapter and the 3-prime adapter is a branch adapter.

31. The method of claim 28 wherein the method further comprises adding a nuclease to extend the intervals formed in step (c), wherein the nuclease has single-strand exonuclease activity.

32. The method of claim 31, wherein the at least some of the primers are RNA primers, and wherein the nuclease is an RNAse H, wherein the RNAse H digests the RNA primers, thereby extending the intervals.

33. The method of claim 28, wherein the primer-binding sequence is located 3-prime to the complement of the barcode sequence in step (a),
wherein the exonuclease has a 3′→5′ exonuclease activity, and
wherein the barcode sequence in each of the set of adaptered fragments is located 5-prime relative to the target sequence fragment.

34. The method of claim 28, wherein the primer-binding sequence is located 5-prime relative to the complement of the barcode sequence in step (a),
wherein the exonuclease has a 5′→3′ exonuclease activity, and
wherein the barcode sequence is 3-prime relative to the target sequence fragment in each of the adaptered fragments.

35. The method of any one of the preceding claims, wherein the both the 5-prime adapter and the 3-prime adapter are in solution.

36. The method of claim 35, wherein the reaction is free of solid supports.

37. The method of any one of the preceding claims, wherein the target sequence has a length between 500 bases to 50 kilobases.

38. The method of claim 30, wherein the branch adapter comprises a double-stranded blunt end comprising a 5′ terminus of one strand and a 3′ terminus of the complementary strand and wherein the 5′ terminus of the strand in the double-stranded blunt end is ligated to the 3′terminus of at least one of the extended primers via branch ligation.

39. The method of claim 30, wherein the L-adapter comprises 1-10 degenerated bases at 3-prime.

40. A method for preparing a plurality of nested sets of adaptered fragments, wherein each adaptered fragment is a single-stranded nucleic acid comprising a target sequence fragment having a first end and a second end, a 5-prime adapter sequence, a 3-prime adapter sequence, a primer-binding sequence, and a complement of a barcode sequence,
wherein in each nested set of adaptered fragments, the target sequence fragments have identical nucleotide sequences at a first end and differ from each other at a second end, such that each nested set of adaptered fragments comprises a plurality of target sequence fragments having different length,
wherein the first end is closer to the barcode sequence than the second end,
wherein the method comprises
(a) providing a barcoded fragment comprising a barcode sequence, a target sequence, and a primer binding sequence, wherein the barcoded fragment is immobilized on a bead at one terminus,
(b) annealing a primer comprising the 5-prime adapter sequence to the primer-binding sequence in the barcoded fragment,
wherein the 5-prime adapter sequence comprises i) a complement of the barcode sequence, and ii) a primer sequence complementary to the primer binding sequence in the barcoded fragment,
(c) extending the primer to produce an extended primer comprising a target sequence fragment and a complement of the barcode sequence,
(d) contacting the extended primer with a branch adapter comprising the 3-prime adapter sequence to produce an adaptered fragment,
(e) separating the adaptered fragment from the barcoded fragment that remains immobilized on the bead, and
(f) repeating steps (b)-(e) for one or more cycles under extension-controlling conditions to produce one or more adaptered fragments,
wherein the adaptered fragment generated from step (e) and the adaptered fragments generated from step (f) and constitute the nested set of adaptered fragments, and
wherein the adaptered fragments in each nested set comprise target sequence fragments having different length.

41. The method of claim 40, wherein the primer is extended under extension-controlling conditions with uracils in one or more cycle of extensions s to produce the extended primer, thereby producing the adaptered fragment incorporating the uracils at 5 prime portion of the target sequence fragment,
(g) contacting the adaptered fragment with an enzyme that removes the incorporated uracils, thereby creating at least one interval flanked by an exposed 3-prime terminus and an exposed 5-prime terminus of the adaptered fragment,
(h) ligating an internal branch adapter to the exposed 3-prime terminus in the at least one interval and ligating an L-adapter to the exposed 5-prime terminus in the interval, and
(i) joining the internal branch adapter that has been ligated to the exposed 3-prime terminus and the L-adapter that has been ligated to the exposed 5-prime terminus in step (h), thereby creating a shortened adaptered fragment,
thereby producing a set of shortened adaptered fragments comprising shortened target sequence fragments having sequences that correspond to different regions of the target sequence and the different regions are overlapping.

42. The method of claim 41, wherein ligating the internal branch adapter and the L-adapter comprises contacting the internal branch adapter and the L-adapter with an splint oligonucleotide,
wherein the a splint oligonucleotide comprises a 5-prime portion that is complementary to a sequence in the internal branch adapter and a 3-prime portion that is complementary to the L-adapter,
thereby the splint oligonucleotide hybridizes to the internal branch adapter via the 5-prime portion and the splint oligonucleotide hybridizes to the L-adapter via the 3-prime portion, thereby ligating the internal branch adapter and the L-adapter.

43. A method for preparing a plurality of sets of adaptered fragments,
wherein each adaptered fragment is a single-stranded nucleic acid comprising a target sequence fragment having a first end and a second end, a 5-prime adapter sequence, a 3-prime adapter sequence, a primer-binding sequence, and a complement of a barcode sequence,
wherein the method comprises
(a) providing a barcoded fragment comprising a barcode sequence, a target sequence, and a primer binding sequence, wherein the barcoded fragment is immobilized on a bead at one terminus,
(b) annealing a primer comprising the 5-prime adapter sequence to the primer-binding sequence in the barcoded fragment,
wherein the 5-prime adapter sequence comprises i) a complement of the barcode sequence, and ii) a primer sequence complementary to the primer binding sequence in the barcoded fragment,
(c) extending the primer to produce an extended primer comprising a target sequence fragment and the complement of the barcode sequence,
(d) contacting the extended primer with a first branch adapter comprising a 3-prime portion comprising a degenerate sequence region,
thereby forming a first extension product comprising the degenerate sequence region at the 3-prime portion,
wherein the 3-prime portion is hybridized to the barcoded fragment through the degenerate sequence region,
(e) extending the 3-prime portion of the first extension product to generate a second extension product, and
(f) contacting the second extension product with a second branch adapter to produce the adaptered fragment.

44. The method of claim 43, wherein the method further comprises
(g) denaturing to separate the adaptered fragment from the barcoded fragment.

45. The method of claim 44, wherein the method further comprises repeating steps (b)-(g) for one or more cycles under extension-controlling conditions to produce one or more adaptered fragments.

46. A DNA complex comprising a plurality of fragments hybridized to one or more monomers of a DNA concatemer, wherein the plurality of fragments are separated by intervals,
wherein each of the plurality of fragments comprises a barcode sequence and a target sequence fragment having a first end and a second end,
wherein the target sequence fragments of the plurality of fragments have identical nucleotide sequences at the first end and differ from each other by truncations at the second end, such that the target sequence fragments of the plurality of fragments have different length.

47. The DNA complex of claim 46, wherein each of the plurality of fragments is ligated to an L-adapter at 5-prime terminus and a branch adapter at 3-prime terminus.

48. A DNA complex comprising
(a) a barcoded fragment immobilized on a solid support,
wherein the barcoded fragment comprises a barcode sequence and a target sequence, and
(b) a polynucleotide hybridized to the barcoded fragment,
wherein the polynucleotide comprises a 5-prime portion comprising a complement of the barcode sequence, a 3-prime portion comprising a target sequence fragment,
wherein the 5-prime portion and the 3-prime portion are annealed to the barcoded fragment, leaving a middle portion not annealed to the barcoded fragment, thereby forming a bubble.

49. A plurality of DNA complexes of any one of claims 46-48, wherein the DNA complexes share the same barcode sequence.

50. A composition comprising a nested set of adaptered fragments each comprising a barcode sequence and a target sequence fragment having a first end and a second end, a 5-prime adapter sequence, and a 3-prime adapter sequence,
wherein the target sequence fragments have identical nucleotide sequences at the first end and differ from each other by truncations at the second end, such that the nested set of adaptered fragments comprises a plurality of target sequence fragments having different length, and
wherein the nested set of adaptered fragments share same barcode sequence.
